(19) United States
(12) Patent Application Publication
(10) Pub. No.: US 2025/0340602 A1
(43) Pub. Date: Nov. 6, 2025

(54) OMNIVOROUS BAKER'S YEAST AND RELATED METHODS

(71) Applicant: Trustees of Tufts College, Medford, MA (US)

(72) Inventors: Nikhil U. Nair, Medford, MA (US); Vikas Trivedi, Medford, MA (US); Sean F. Sullivan, Medford, MA (US); Venkatesh Endalur Gopinarayanan, Medford, MA (US)

(21) Appl. No.: 19/199,244

(22) Filed: May 5, 2025

Related U.S. Application Data
(60) Provisional application No. 63/642,364, filed on May 3, 2024.

(51) Int. Cl. C07K 14/395; C12N 15/81

(52) U.S. Cl. CPC C07K 14/395; C12N 15/81

(57) ABSTRACT

Disclosed herein are engineered proteins and methods of engineering microbial organisms. An engineered protein is disclosed. The engineered protein is a variant of Gal3p and the variant of Gal3p is fully activated. The variant of Gal3p may possess a conformational change corresponding to galactose-bound Gal3p. The variant of Gal3p may be Gal3p^{MC}. Microbial organisms and methods of engineering microbial organisms for growth on non-native substrates are disclosed. The microbial organism may be Saccharomyces cerevisiae. The method comprises synthetically activating the GAL response system in the microbial organism. The method may comprise expressing Gal3p^{MC} in Saccharomyces cerevisiae.

### CROSS-REFERENCE TO RELATED APPLICATIONS

The present application claims priority to U.S. Provisional Patent Application No. 63/642,364, filed May 3, 2024. The entire contents of which are hereby incorporated by reference.

### STATEMENT REGARDING FEDERALLY SPONSORED RESEARCH

This invention was made with government support under grants HD091798 and HD105934 awarded by the National Institutes of Health and grants 1421972 and 1935354 awarded by the National Science Foundation. The government has certain rights in the invention.

### SEQUENCE LISTING

A Sequence Listing accompanies this application and is submitted as an xml file of the sequence listing named “166118_01528.xml” which is 28,997 bytes in size and was created on May 5, 2025. The sequence listing is electronically submitted via Patent Center and is incorporated by reference herein in its entirety.

### BACKGROUND OF THE INVENTION

The adoption of abundant and renewable substrates as inputs for biotechnology will be essential for creating a sustainable, circular bioeconomy, but the inability of industrially important microbes, like Saccharomyces cerevisiae, to utilize many potential substrates poses a major hurdle to realizing this goal. The existing paradigm for engineering the assimilation of non-natives substrates (i.e., synthetic heterotrophy) in yeast begins with the identification and constitutive overexpression of catabolic genes that enable the substrate to enter central carbon metabolism (CCM) where it is expected to be transformed into the key primary metabolites used for growth and biosynthesis. However, this method ignores the tight regulation of CCM and how these resources are distributed to accomplish cellular objectives. Moreover, existing efforts are often focused on a specific substrate or small set of substrates. As such, there is a need in the art to identify methods of engineering microbes to be omnivorous in their substrate compatibility.

### BRIEF SUMMARY OF THE INVENTION

In some aspects, the present disclosure provides an engineered protein, wherein the engineered protein is a variant of Gal3p. The variant of Gal3p is fully activated. The variant of Gal3p may possess a conformational change corresponding to galactose-bound Gal3p. In some aspects, the variant of Gal3p comprises SEQ ID NO: 1 or a sequence having at least 80% identity thereto. In some aspects, the variant of Gal3p is Gal3pMC. The engineered protein may activate the galactose regulon, and the galactose regulon may be activated by indirect action. The engineered protein may allow a microbial cell comprising or expressing the engineered protein to grow on a non-native substrate.

In some aspects, the present disclosure provides a nucleic acid construct encoding an engineered protein described herein. In some aspects, the nucleic acid construct comprises SEQ ID NO: 15 or a sequence having at least 80% identity thereto.

In some aspects, the present disclosure provides a microbial cell comprising or expressing an engineered protein described herein and/or a nucleic acid construct described herein.

In some aspects, the present disclosure provides a multicellular microbial organism comprising at least one microbial cell described herein. The microbial cell may be a yeast, and the yeast may be Saccharomyces cerevisiae. The microbial cell grows in an inducer-independent manner.

In some aspects, the present disclosure provides a method of engineering a microbial organism for growth on a non-native substrate, the method comprising synthetically activating the GAL response system in the microbial organism. In some aspects, a semi-synthetic GAL regulon activates the GAL response system. The method may further comprise synergizing activation of the semi-synthetic GAL regulon with an optimized upstream heterologous metabolic module. The optimized upstream heterologous metabolic module may comprise a deletion of at least one negative effector gene, the at least one negative effector gene may minimize oxidation of substrate pentoses to pentitols, and, in some aspects, the at least one negative effector gene is GRE3. In some aspects, the optimized upstream heterologous metabolic module comprises an overexpression of at least one positive effector gene, the at least one positive effector gene may be a pentose metabolic gene, and in some aspects, the at least one positive effector gene is selected from the group consisting of TAL1 (encoding SEQ ID NO: 16), GAL2 (encoding SEQ ID NO: 17), araBAD, which comprises the araA (encoding SEQ ID NO: 18), araB (encoding SEQ ID NO: 19), and araD (encoding SEQ ID NO: 20) genes, and XYLA*3-XKS1. In some aspects, the method does not comprise modifying catabolic genes in the microbial organism.

In some aspects, the present disclosure provides a method of growing a microbial organism on a non-native substrate, wherein the method comprises expressing an engineered protein disclosed herein and/or a nucleic acid construct described herein in the microbial organism. The microbial organism may grow in an inducer-independent manner. The non-native substrate may be a sugar, and, in some aspects, the sugar is selected from the group consisting of arabinose, xylose, cellobiose, and raffinose. The sugar may be a sugar which excludes at least one of glucose and sucrose. In some aspects, the microbial organism is a yeast and may be Saccharomyces cerevisiae. In some aspects, the method does not comprise modifying catabolic genes in the microbial organism.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A, 1B, and 1C show expression from GAL-inducible promoters is coordinated with growth. (A) Simplified regulatory overview of the GAL regulon. The binding of galactose to sensor protein Gal3p enables it to relieve the repression of Gal80p on Gal4p via protein-protein interactions. Gal4p binds to its cognate Upstream Activating Sequences (UAS) to directly transcribe galactose catabolic genes as well as to indirectly reshape global metabolism for rapid growth. The regulon is also repressed in the presence of excess glucose, a regulatory logic that ensures the regulon is activated only in the presence of its target carbon source but not when a preferred carbon source is present. Expression of EGFP in wild-type strain W303-1a from (B) strong constitutive promoters (TEF1p, TPI1p, TDH3p, GPM1p) and (C) GAL-inducible promoters (GAL1p, GAL7p, GAL10p, GAL3p, GAL80p) at different time points during cultivation on galactose overlaid with cell density (OD₆₀₀). Each data point represents the average of three (for OD₆₀₀ values) or four (for fluorescence) biological replicates±sd.

FIGS. 2A, 2B, and 2C show sensor protein variant Gal3pMC (SEQ ID NO: 1) enables robust GAL regulon activation in an inducer-independent manner. (A) Schematic of fluorescent reporter assay used to quantify GAL regulon activation by different GAL3 variants. Cells expressing a given variant (ΔGAL3, Gal3pWT (SEQ ID NO: 2), or Gal3pMC) are grown in the presence of both native and non-native substrates. Regulon activation drives EGFP expression from the GAL-inducible promoter, GAL1p. (B) Comparison of normalized fluorescence resulting from interaction between GAL3 variant and carbon source (2% w/v) using RAF to support growth on substrates GAL, ARA, XYL, and CEL. SUC and GLU are repressing substrates. (C) Expression of EGFP from TEF1p and GAL1p during growth on galactose in a strain (SFS6) expressing Gal3pMC to activate the regulon. Each data point represents the average of 5-6 biological replicates ±sd.

FIGS. 3A, 3B, 3C, and 3D show metadynamics simulations that identify energetically favorable conformations. (A) Two collective variables (CV) were defined to enable energetic exploration of the Gal3p conformational landscape: CV1 as the distance between C1 and C2, the centers of mass of the two moving domains, and CV2 as the angle formed between C1, C3 (the hinge region center of mass), and C2. Calculating the energy associated with different values of CV1 and CV2 enabled construction of the free energy surfaces (FES) for the (B) WTGal− (W), (C) WTGal+(G), and (D) MCGal− (M) systems. The subscripts “m”, “c”, and “o”, corresponding to misfolded (collapsed), closed, and open states, respectively, are used to associate prominent energetic minima with distinct conformations of Gal3p variants. Significantly, the most favorable conformations in the WTGal+ and MCGal− systems correspond to a closed state that likely favors interaction with Gal80p and thus regulon activation.

FIGS. 4A and 4B show docking interactions between Gal3p and Gal80p. (A) Detailed representation of the interface between Gal3p (purple) and Gal80p (pink) complex. The residues involved in the interface are highlighted in the call-out. (B) The average binding energy between Gal3p and Gal80p was calculated by sampling every 0.02 ns from the docked Gal3p-Gal80p complexes obtained through protein-protein docking study. A statistical analysis was performed on the average of the four binding energy values using ANOVA to determine whether there was a significant difference in the four sets of calculated binding energies. The results of the analysis showed an F-value of 2137 with 3 degrees of freedom and p-value of <0.0001, indicating a significant difference between all four.

FIGS. 5A, 5B, 5C, 5D, 5E, 5F, and 5G show comparisons of the fitness of substrate-specific and substrate-agnostic approaches to GAL regulon activation. (A) Schematic illustrating different ways of activating the GAL regulon in cells carrying xylose catabolic genes under the control of GAL-responsive promoters. Gal3pSyn4.1 (SEQ ID NO: 3) requires induction by xylose, Gal3pMC does not require an inducer but maintains the native regulatory architecture, while ΔGAL80 effects inducer-independent activation by removing native regulatory elements. Growth of the three systems in defined (SC) medium containing 2% of (B) sucrose, (C) glucose, or (D) raffinose reveals that a de-regulated approach (ΔGAL80) reduces fitness on native carbon sources. (E) Comparing growth of the three GAL regulon activation systems on the non-native sugar xylose after being subcultured from sucrose. Comparison of constitutive (CONS) and regulon (REG) approaches to growth in complex media containing 2% of (F) arabinose or (G) cellobiose as sole carbon sources. All data points represent the average of three biological replicates ±sd.

FIGS. 6A, 6B, 6C, 6D, and 6E show Gal3pMC enables rapid and complete co-utilization of multiple non-native substrates. (A) Placing REG plasmids for the catabolism of cellobiose, xylose, and arabinose into separate strains represents a Consortium approach to co-utilization while placing all three plasmids into a single strain represents a Consolidated approach. (B) Cell density (black) and percent residual substrate of cellobiose (yellow), xylose (light blue), and arabinose (pink) of Consortium (dotted lines) and Consolidated (solid lines) approaches to co-utilization in complex media containing 1% of each substrate. Incomplete pentose utilization motivated the exploration of all six possible pairings between GAL-inducible promoters (GAL1p, GAL10p, and GAL7p in order) and the catabolic genes for (C) arabinose (A=L-arabinose isomerase, B=L-ribulokinase, D=L-ribulose-5-phosphate-4-epimerase) and (D) xylose utilization (XI=xylose isomerase, XKS=D-xylulokinase, (p=no gene). Promoter-gene pairings used in subsequent co-utilization experiments are indicated by (*). (E) Re-testing a Consolidated approach to co-utilization using expression-balanced pentose plasmids results in complete utilization of all three substrates. Data points represent the average of three (panel B) or two (panel E) biological replicates ±sd.

FIGS. 7A, 7B, 7C, 7D, 7E, and 7F show the implementation of a semi-synthetic regulon design for synthetic heterotrophy (A) The GAL regulon, synthetically activated by Gal3pSyn4.1, regulates GAL-responsive genes as well as hundreds of downstream genes to promote growth. The traditional approach of constitutive overexpression cannot activate downstream genes, which leads to starvation response and slow growth. (B) Dose-dependent GAL1p-EGFP activation by arabinose of strain expressing Gal3pWT or Gal3−pSyn4.1 in and Gal2pWT or Gal2p2.1. (C) Comparison of GAL1p activation by 20% of native sugar, galactose, and non-native sugars—xylose and arabinose. (D-F) Genetic structure of semi-integrant REG and CONS strain backgrounds.

Growth curve of REG and CONS strains on E) arabinose and F) xylose when transformed with respective arabinose and xylose isomerase pathway genes, respectively. The high variability is growth rate is partly contributed by variabilities associated with using 2 mm-based high- copy plasmids for multigene expression. All cell growth assays done in triplicates (or more) and are presented as mean±SD. REG and CONS strains use GAL-responsive and constitutive promoters, respectively, for all genes indicated (Tables 1 and 2).

FIGS. 8A, 8B, and 8C show systems analysis to identify intrinsic limitations in yeast for growth on pentoses (A) Differential expression volcano plot for transcriptomic comparison of strains grown individually on galactose or pentose sugars (xylose or arabinose) with the number of genes relatively upregulated in each condition overlaid. (B) Plots of Betweenness Centrality (BC) (arbitrary units) vs. significance (−log 10P) for the subset of significantly (p<0.05) differentially expressed genes demonstrate how native gene targets for deletion were identified using three yeast gene regulatory networks (GRNs): EGRIN (top), YEASTRACT (center), and CLR (bottom). Genes were categorized as either, scoring highest in BC in a given network and picked for deletion (red), not picked for deletion (gold), identified as deletion target in another network (purple), or all remaining significant nodes (gray). (C) 24 highly connected genes identified via differential gene expression and gene regulatory network analyses (with p<0.05) as targets for deletion to improve growth on pentoses.

FIGS. 9A, 9B, 9C, and 9D show the effect of gene knockouts on growth phenotype (A) Fitness map of knockout library of 24 genes identified from the mutual information network. The scale represents fitness ranging from blue (negative) to orange (positive). Growth rates of KOs with positive fitness on (B) galactose, (C) xylose (GAL1p- XYLA*3 GAL10p-XKS1), (D) arabinose (GAL1p-araB GAL10p-araA GAL7p-araD). Glc=glucose, Gal=galactose, Xyl=xylose, Ara=arabinose. The growth rates were normalized from the average of at least two biological replicates.

FIGS. 10A, 10B, 10C, 10D, 10E, 10F, 10G, 10H, 10I, and 10J show optimizing extrinsic (upstream) factors to enhance growth of yeast on pentoses (A) Comparison of growth performance of different promoter-gene combination. (B-E) Gene expression analysis of the six strains by qPCR. Pearson correlation coefficient (p) between growth rate and gene expression levels of (C) araA:araB, (D) araB:araD, and (E) araA:araD, respectively. (G) Enrichment of mutagenic araBAD library in SC+Ara. (H) Fitness heatmap of strain dynamics during enrichment on arabinose, revealed by sequencing barcodes. (I) Growth curves of top variants from the directed evolution study with parental control strain (ADB) as reference, and (J) qPCR expression profile of ara-BAD genes encoded in plasmids isolated from top performing strains isolated after directed evolution. All cell growth assays and qPCRs were done in triplicates (or more) and the data are presented as mean±SD.

FIGS. 11A, 11B, 11C, 11D, and 11E show the effect of gene deletions on growth and stress-response phenotypes (A) Growth rate of the optimized plasmids in KO-strains. Fitness of KOs in presence of individual stressors when grown on (B) sucrose and (C) arabinose or in the mixture of stressors on (D) sucrose and (E) arabinose. The relative fitness is expressed as growth of deletion strains over the fitness of parental strain under the same conditions. NaCl=sodium chloride; NaAc=sodium acetate; Fur=furfural; 5HMF=5-hydroxymethylfurfural; Vald=vera-traldehyde. ½x, ¼x, ⅛x=serial dilutions of the five stressors combined. Xyl* =GAL10p-XYLA*3 GAL7p-XKS1; Ara* =GAL1p-araA GAL10p-araB GAL7p-araD in respective REG backgrounds. The growth rates were normalized from the average of at least two biological replicates.

FIGS. 12A and 12B show expression dynamics of constitutive and GAL-inducible promoters during growth on glucose. Expression of EGFP in wild-type strain W303-1a from (A) strong constitutive promoters (TEF1p, TPI1p, TDH3p, GPM1p) and (B) GAL-inducible promoters (GAL1p, GAL7p, GAL10p, GAL3p, GAL80p) at different time points during cultivation on glucose. While expression profile from constitutive promoters is similar to that observed during growth on galactose, GAL-inducible promoters are repressed via carbon catabolite repression. Each data point represents the average of four biological replicates ±sd.

FIGS. 13A and 13B show an initial characterization of autoactivating Gal3p mutants. (A) Activation of GAL1p-EGFP using partial autoactivating mutants identified by Blank et al. (1997). None of the 3 variants tested including Gal3p^{F509P} (SEQ ID NO: 4), Gal3p^{F237Y} (SEQ ID NO: 5), and Gal3p^{F237Y+S509P} (SEQ ID NO: 6) fully activate GAL1p to the same level as Gal3p^{WT} does with galactose. (B) Combination of F237Y with Gal3p^{Syn4.1} yielded Gal3p^{MC} that activates more strongly than F237Y alone. All experiments use 2% (w/v) sugar. GLU=Glucose, SUC=sucrose, GAL=galactose, ARA=arabinose, XYL=xylose. Each data point represents the average of three biological replicates ±sd.

FIG. 14 shows a comparison of fluorescence (normalized for cell density) resulting from interaction between GAL3 variant and carbon source (2% w/v) using sucrose to support growth on substrates (GAL/ARA/XYL/CEL) for which strain VEG16 lacks catabolic genes. Each data point represents the average of four biological replicates ±sd. GAL=galactose, ARA=arabinose, XYL =xylose, CEL=cellobiose, ETH/GLY=ethanol/glycerol, SUC=sucrose, GLU=glucose.

FIG. 15 shows the structure of wild-type Gal3p in complex with Gal80p. Gal3p^{MC} was constructed by combining mutations identified as conferring robust xylose induction (D68N, V69M, A109V, 1271L, highlighted in green) in variant Gal3p^{Syn4.1} with a mutation (F237Y, highlighted in red) that confers a partial autoactivating phenotype.

FIGS. 16A, 16B, 16C, 16D, 16E, 16F, 16G, 16H, 16I, 16J, and 16K show observations of 1000 ns scale molecular dynamics (MD) simulations. (A) Structural representation of Gal3p in the closed conformation with dotted lines representing the distance between residues L370 and D104, found on the opposing ‘moving’ domains (cyan- and pink-colored regions), that is monitored during the simulation. (B) Time series of the distance measured between residues L370 and D104 records domain movement across 1000 ns of simulations. Volume of active site pocket (depicted as blue spheres) calculated for the last frame of the simulation for the (D) WT^{Gal-}, (E) WT^{Gal+}, and (F) MC^{Gal-} systems. Cross correlation studies were used to derive residue-residue correlation heatmaps for (G) WT^{Gal-}, (H) WT^{Gal+}, and (I) MC^{Gal-}. Cross-correlation maps showed an intense residue-residue correlation between residues 90-250 and residues 250-350 in WT^{Gal-} that was not observed in the other two systems. Porcupine plots constructed from a principal component analysis (PCA) of the dynamics from the MD trajectories of (D) WT^{Gal-}, (E) WT^{Gal+} and (F) MC^{Gal-} use spikes to show the intensity and direction of the principal component of motion for each residue. WT^{Gal-} showed approximately three-fold and four-fold greater motion relative to WT^{Gal+} and MC^{Gal-}, respectively. Regions showing increased dynamics in the PCA of WT^{Gal-} correspond to the same regions showing defined residue-residue correlation from the cross-correlation studies.

FIGS. 17A, 17B, and 17C show residue-residue interaction networks were generated for the (A) WT^{Gal-}, (B) WT^{Gal+}, and (C) MC^{Gal-} systems using the NetworkView plugin in Visual Molecular Dynamics. Interaction networks between the static hinge (grey) region and the moving (cyan and pink) regions are visualized using green and red ball-and-stick models, respectively. Spheres represent nodes that are residues while edges represent the interaction between the residues with the thickness of the edge indicating the strength of interaction. The large number of interaction networks seen in WT^{Gal-} likely contributes to the formation the of compact, ‘collapsed’ structure observed during the molecular dynamics simulations. In contrast, the WT^{Gal+} and MC^{Gal}systems exhibit much sparser interaction networks.

FIG. 18 shows a comparison of growth of the three GAL regulon activation systems on the non-native sugar xylose after being sub-cultured from glucose. Gal3p^{Syn4.1} requires induction by xylose, Gal3pMC does not require an inducer but maintains the native regulatory architecture, while ΔGAL80 effects inducer-independent activation by removing native regulatory elements. All data points represent the average of three biological replicates ±sd.

FIGS. 19A and 19B show data demonstrating use of Gal3p^{MC} to rapidly apply a regulon approach to growth on the non-native sugars, arabinose and cellobiose. Growth of CONS and REG strains in defined media containing with in (A) 2% arabinose or (B) 2% cellobiose as the sole carbon source. All data points represent the average of three biological replicates ±sd.

FIGS. 20A and 20B show relative expression of catabolic genes in strains shown in FIG. 16. Cell-density normalized expression of a fluorescent protein (EGFP) from the representative promoters GAL1p and TEF1p used to control catabolic gene expression in REG and CONS strains, respectively, was quantified during exponential growth (t=14 hours). These values serve as a proxy for the relative catabolic gene expression occurring in strains grown on xylose utilizing (A) three different approaches—substrate-specific (Gal3p^{Syn4.1}), substrate-agnostic (Gal3p^{MC}), and de-regulated (ΔGAL80) GAL regulon activation. (B) CONS and REG approaches to growth on arabinose and cellobiose as sole carbon sources. Each data point represents the average of at least three biological replicates ±sd.

FIGS. 21A, 21B, and 21C show cell density (black) and percent residual substrate of cellobiose (yellow), xylose (light blue), arabinose (pink), and both pentoses combined (purple) of Consolidated approach to co-utilization in complex media containing (A) 0.7%-0.7%-0.7%, (B) 1%-0.5%-0.5%, or (C) 0.4%-0.8%-0.8% CEL-XYL-ARA (w/v). All data points represent the average of two biological replicates ±sd.

FIGS. 22A and 22B show (A) sequence alignment of the crystal structure (3V2U; SEQ ID NO: 8) of the closed state of Gal3p and the modelled structure of Gal3p protein (SEQ ID NO: 7). (B) 3D ribbon representation of the modelled structure of Gal3p protein, with the red loop indicating the modeled region spanning from residues 11 to 15 that was missing in the crystal structure.

FIG. 23 shows a Ramachandran plot that was used to validate the model generated from SwissModel. This plot confirms that none of the residues in the model are in disallowed regions, indicating the high quality of our model.

FIG. 24 shows a comparative analysis was conducted to evaluate the accuracy of the models generated by Swiss-Model in relation to the closed-form crystal structure of Gal3p (PDB ID: 3V2U). The models were superimposed over the crystal structure in ribbon form, and the missing region in the model was highlighted. The Root Mean Square Deviation (RMSD) between the structures was calculated and found to be 0.09, indicating a close resemblance between the modelled and crystal structures.

FIGS. 25A, 25B, 25C, and 25D show the superimposition analysis was conducted to compare the structures of Gal3p with the apo form crystal structure (grey ribbon, PDB ID 3V5R). The structures of WT^{Gal+} (blue), WT^{Gal-} (red), and MC^{Gal-} (green) were obtained from the minimum energy states of metadynamics simulations. The crystal structure of WT^{Gal+} in the closed state (violet, PDB ID 3V2U) was also included in the analysis. The RMSD values of the Ca backbone atoms were calculated to assess the structural differences. The RMSD values for WT^{Gal+}, WT^{Gal-} MC^{Gal-} with respect to the crystal structure in closed state were found to be 2.955, 4.422, and 3.220, respectively.

### DETAILED DESCRIPTION OF THE INVENTION

Before the present invention is described in further detail, it is to be understood that the invention is not limited to the particular embodiments described. It is also understood that the terminology used herein is for the purpose of describing particular embodiments only and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced.

Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10. Further, as used herein, ranges that are between two particular values should be understood to expressly include those two particular values. For example, “between 0 and 1” means “from 0 to 1” and expressly includes 0 and 1 and anything falling inside these values. Also, as used herein “about” means±20% of the stated value, and includes more specifically values of ±10%, ±5%, ±2%, ±1%, and ±0.5% of the stated value.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

Further, the terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

As used herein, the terms “peptide,” “polypeptide,” and “protein,” refer to molecules comprising a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard or unnatural amino acids. The term “amino acid residue” may include alpha-, beta-, gamma-, and delta-amino acids.

Variants or derivatives as contemplated herein may have an amino acid sequence that includes conservative amino acid substitutions relative to a reference amino acid sequence. For example, a variant or derivative peptide, polypeptide, or protein as contemplated herein may include conservative amino acid substitutions and/or non-conservative amino acid substitutions relative to a reference peptide, polypeptide, or protein. “Conservative amino acid substitutions” are those substitutions that are predicted to interfere least with the properties of the reference peptide, polypeptide, or protein, and “non-conservative amino acid substitution” are those substitution that are predicted to interfere most with the properties of the reference peptide, polypeptide, or protein. In other words, conservative amino acid substitutions substantially conserve the structure and the function of the reference peptide, polypeptide, or protein. The following table provides a list of exemplary conservative amino acid substitutions.

| Original | |
|---|---|
| Residue | Conservative Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain: (a) the structure of the peptide, polypeptide, or protein backbone in the area of the substitution, for example, as a beta sheet or alpha helical conformation, (b) the charge or hydrophobicity of the molecule at the site of the substitution, and/or (c) the bulk of the side chain. Non-conservative amino acid substitutions generally disrupt: (a) the structure of the peptide, polypeptide, or protein backbone in the area of the substitution, for example, as a beta sheet or alpha helical conformation, (b) the charge or hydrophobicity of the molecule at the site of the substitution, and/or (c) the bulk of the side chain.

Variants or derivatives comprising deletions relative to a reference amino acid sequence of peptide, polypeptide, or protein are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation of a reference polypeptide or a 5′-terminal or 3′-terminal truncation of a reference polynucleotide).

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number (e.g., SEQ ID NO:1), or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant” or “derivative” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides. A “variant” or “derivative” may have substantially the same functional activity as a reference polypeptide (e.g., glycosylase activity or other activity. Variant or derivative polypeptides as contemplated herein may include variant or derivative polypeptides of SEQ ID NO: 1).

Engineering the utilization of non-native substrates, or synthetic heterotrophy, in proven industrial microbes such as Saccharomyces cerevisiae (which may be referred to as “Baker's yeast” herein and elsewhere) represents an opportunity to valorize plentiful and renewable sources of carbon and energy as inputs to bioprocesses. Activation of the galactose (GAL) regulon, a regulatory structure used by this yeast to coordinate substrate utilization with biomass formation during growth on galactose, during growth on the non-native substrate xylose results in a vastly altered gene expression profile and faster growth compared with constitutive overexpression of the same heterologous catabolic pathway. However, this effort involves the creation of a xylose-inducible variant of Gal3p (Gal3p^{Syn4.1}; SEQ ID NO: 3), the sensor protein of the GAL regulon, preventing a semi-synthetic regulon approach from being easily adapted to additional non-native substrates. Disclosed herein is at least a variant Gal3p^{MC} (metabolic coordinator; SEQ ID NO: 1) that exhibits robust GAL regulon activation in the presence of structurally diverse substrates and recapitulates the dynamics of the native system. Multiple molecular modeling studies suggest that Gal3p^{MC} occupies conformational states corresponding to galactose-bound Gal3p in an inducer-independent manner. Using Gal3p^{MC} to test a regulon approach to the assimilation of the non-native lignocellulosic sugars xylose, arabinose, and cellobiose yields higher growth rates and final cell densities when compared with a constitutive overexpression of the same set of catabolic genes. The subsequent demonstration of rapid and complete co-utilization of all three non-native substrates suggests that Gal3p^{MC}-mediated dynamic global gene expression changes by GAL regulon activation may be universally beneficial for engineering synthetic heterotrophy.

In one aspect, the present disclosure provides an engineered protein, wherein the engineered protein is a variant of Gal3p, wherein the variant of Gal3p is fully activated. As used herein, “engineered protein” may refer to a polypeptide modified to perform a specific function. For example, a protein mutated to conform to a different shape. “Protein” may be used interchangeably with “protein” or “polypeptide”. As used herein, “Gal3p” may refer to the allosteric monomeric protein that activates the GAL genetic switch of Saccharomyces cerevisiae in response to galactose. Gal3p may be used interchangeably with Gal3 or GAL3 which includes the protein or nucleic acid sequence encoding said protein that may act as a transcriptional regulator involved in activation of the GAL family of genes in response to galactose. In particular, Gal3p may form a complex with Gal80p to relieve Gal80p inhibition of Gal4p; Gal3p may bind galactose and ATP but may not specifically have galactokinase activity; GAL3 has a paralog, GAL1, that arose from the whole genome duplication. In particular, Gal3p may be native to yeast and, in particular, may be native to Saccharomyces cerevisiae. As used herein, “fully activated” may refer to the ability of a protein to produce its native effect or the native effect of the protein from which it was derived without the need for sufficient stimuli. For example, Gal3p natively activates the GAL genetic switch of Saccharomyces cerevisiae in response to galactose. Therefore, a fully activated (or “fully active”) Gal3p protein activates the GAL genetic switch in response to substrates other than galactose (that is “non-native substrates”) or to no substrate at all. This may be referred to as “constitutive activity”.

The inventors presently disclose a conformational change in Gal3p upon activation (that is, upon interacting with galactose). However, unlike its parent protein (Gal3p), Gal3p^{MC} possess this active conformational change in an inducer-independent matter (that is, when activated by substrates other than galactose (i.e., “non-native substrates”)). Therefore, in some aspects, the variant of Gal3p possesses a conformational change corresponding to galactose-bound Gal3p. “Galactose-bound” may be used interchangeably with “galactose-activated”. As used herein, “conformational change” may refer to a structural difference between a protein's active and inactive forms and/or a protein's native and derivative forms. The inventors presently disclose one such Gal3p variant that possesses such a conformational change and/or is fully activated. Therefore, in some aspects, the variant of Gal3p comprises SEQ ID NO: 1 or a sequence having at least 80% identity thereto. In some aspects, the variant of Gal3p is Gal3p^{MC} (or “Gal3p^{MC}”). SEQ ID NO: 2 is the amino acid sequence for wild type Gal3p. Gal3p^{MC} comprises the following mutations, relative to SEQ ID NO: 2: D68N, V69M, A109V, F237Y, and 1271L. The disclosed variants of Gal3p may comprise sequences with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more identity to SEQ ID NO: 1 and D68N, V69M, A109V, F237Y, and 1271L substitution mutations with reference to SEQ ID NO: 2.

The inventors presently disclose that full activation of Gal3p (in the case of Gal3p^{MC}, for example) activates the galactose regulon. Therefore, in some aspects, the engineered protein activates the galactose regulon. As used herein, “activates” may refer to the ability of a protein to affect its downstream effectors, and, therefore, produce its native effect. As used herein, “galactose regulon” may refer to the regulatory mechanism by which a microbial organism responds to galactose. In particular, the galactose regulon responds to galactose by inducing a conformational change in Gal3p which then forms a complex with Gal80p thus relieving Gal80p's inhibition of Gal4p which then acts as a transcription factor, ultimately inducing growth. As such, a skilled practitioner will appreciate the use of engineered proteins disclosed herein and the methods of engineering microbial organisms and cells disclosed herein at least in that this regulon approach to the assimilation of the non-native lignocellulosic sugars xylose, arabinose, and cellobiose yields higher growth rates and final cell densities when compared with a constitutive overexpression of the same set of catabolic genes. Therefore, in some aspects, the engineered protein allows a microbial cell comprising or expressing the engineered protein to grow on a non-native substrate.

The inventors presently disclose the direct action of Gal3p^{MC} (that is, the activation of the galactose regulon as discussed above); however, they also disclose the indirect action of Gal3p^{MC} in response to galactose. For example, the inventors found that Gal3p^{MC} expression results in differential expression of genes related to galactose catabolism. Therefore, in some aspects, the galactose regulon is activated by indirect action. As used herein, “indirect action” may refer to the relative upregulation of genes associated with cell division and mitochondrial biogenesis and downregulation of genes that may act as negative effectors of growth.

In another aspect, the present disclosure provides a nucleic acid construct encoding an engineered protein described herein. As used herein, “nucleic acid construct” may refer to an engineered nucleic acid (that is, a nucleic acid modified to perform a specific function or encode a specific protein). Nucleic acid constructs may be in the form of plasmids or vectors. Nucleic acid constructs may alternatively take the form of genomic nucleic acids. In some aspects, the nucleic acid construct comprises SEQ ID NO: 15 or a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more identity to SEQ ID NO: 15.

In another aspect, the present disclosure provides, a cell engineered to express and/or comprise an engineered protein described herein and/or a nucleic acid construct described herein. As used herein, “engineered to comprise” may refer to modifications made to ensure the expression of a particular protein.

In another aspect, the present disclosure provides a microbial organism comprising a cell described herein. Saccharomyces cerevisiae have been shown to form multicellularity (Fisher and Regenberg, 2019, “Multicellular group formation in Saccharomyces cerevisiae”, roc. R. Soc. B.28620191098; Opalek and Wloch-Salamon, 2020, “Aspects of Multicellularity in Saccharomyces cerevisiae Yeast: A Review of Evolutionary and Physiological Mechanisms”, Genes (Basel), 11(6):690). Therefore, in some aspects, the microbial organism is a multicellular organism comprising at least one microbial cell described herein. Examples of microbial organisms include, but is not limited to, unicellular and multicellular yeast.

In another aspect, the present disclosure provides a microbial organism comprising an engineered protein described herein. In some aspects, the microbial organism is a yeast, and, in some aspects, the yeast is Saccharomyces cerevisiae. The inventors presently disclose that the expression of Gal3p^{MC} in Baker's yeast results in omnivorous behavior of the yeast (that is, the yeast is able to grow on multiple different substrates, or “inducers”). Therefore, in some aspects, the microbial organism grows in an inducer-independent manner. As used herein, “inducer-independent manner” may refer to the ability of a microbial organism to grow, for example, in an environment in which the microbial organism would not normally grow due to its dependence on a particular substrate. For example, Baker's yeast may require (i.e., depend on) galactose to grow adequately. However, Baker's yeast growing in an inducer-independent manner would be able to grow on substrates other than galactose (for example, arabinose). “Substrate” may refer to the environment on which a microbial organism grows and may include cell culture conditions.

In another aspect, the present disclosure provides a method of engineering microbial organisms for growth on a non-native substrate, wherein the method comprises synthetically activating the GAL response system on the non-native substrate. As used herein, “non-native substrate” may refer to a substrate on which the microbial organism would not normally grow. As used herein, “synthetically activating” may refer to the activation of a regulon (for example, the galactose regulon) by non-native means (for example, a protein variant). For example, the galactose regulon can be synthetically activated by the Gal3p^{MC} variant. As used herein, “GAL response system” may refer to the response of a microbial organism to galactose and may include, but is not limited to, the activation of the galactose regulon. Therefore, in some aspects, a semi-synthetic GAL regulon activates the GAL response system. As used herein, “a semi-synthetic GAL regulon” may refer to the galactose regulon wherein one or more components comprise a protein variant or are synthetically activated. Means of achieving regulon activation are disclosed herein and in Endalur Gopinarayanan and Nair (2018, “A semi-synthetic regulon enables rapid growth of yeast on xylose. Nat Commun 9, 1233), which is incorporated by reference herein in its entirety.

In some aspects, the method further comprises synergizing activation of the semi-synthetic GAL regulon with an optimized upstream heterologous metabolic module. As used herein, “synergizing activation” may refer to the activation of a pathway through multiple means that act in synergy (i.e., in a multiply beneficial manner that produces a greater effect than adding the multiple components separately). For example, synthetically activating the galactose regulon while inducing expression of positive effector genes and/or reducing expression of negative effector genes may provide synergizing activation of the GAL response. As used herein, “optimized upstream heterologous metabolic module” may refer to the combined downregulation and/or upregulation of a gene or genes that direct non-native substrates to central carbon metabolism. The inventors presently disclose genetic interventions through traditional/systems metabolic engineering that prune cellular metabolic and/or regulatory networks to improve strain performance which often lead to pleiotropic defects with mid-to-severe fitness costs. In some aspects, the optimized upstream heterologous metabolic module comprises a deletion of at least one negative effector gene. In some aspects, the at least one negative effector gene minimizes oxidation of substrate pentoses to pentitols, and, in some aspects, the at least one negative effector gene is GRE3. In some aspects, the optimized upstream heterologous metabolic module comprises an overexpression of at least one positive effector gene, and, in some aspects, the at least one positive effector gene is a pentose metabolic gene. In some aspects, the at least one positive effector gene is selected from the group consisting of TAL1, GAL2, araBAD, and XYLA*3-XKS1. XYLA*3 corresponds to XYLA protein (SEQ ID NO: 21) containing six mutations (E15D, E114G, E129D, T142S, A177T, and V433I) that exhibited a 77% increase in enzymatic activity. XYLA*3-XKS1 may comprise co-expression of XYLA*-3 and XKS1. As used herein, “negative effector gene” may refer to a gene that, when expressed, deters growth in the absence of a microbial organism's native substrate. As used herein, “positive effector gene” may refer to a gene that, when expressed, induces growth in the presence of a microbial organism's native substrate. As used herein, “pentose metabolic gene” may refer to a gene that induces metabolism of pentoses. Exemplary pentoses include, but are not limited to, arabinose, xylose, and ribose.

Although the inventors have identified synergy between regulon-based intervention and genetic intervention, it is not to be misunderstood that regulon-based intervention is insufficient to improve on microbial organism or cell growth on non-native substrates alone. Therefore, in another aspect, the present disclosure provides a method of engineering microbial organisms for growth on a non-native substrate, wherein the method the method does not comprise modifying catabolic genes in the microbial organism.

In another aspect, the present disclosure provides a method of growing a microbial organism on a non-native substrate, wherein the method comprises expressing an engineered protein described herein or a nucleic acid construct described herein in the microbial organism. In some aspects, the microbial organism grows in an inducer-independent manner.

In some aspects, the non-native substrate is a sugar. As used herein, “sugar” may refer to carbohydrates of the general formula Cₙ(H₂O)ₙ. Exemplary sugars include, but are not limited to, arabinose, xylose, cellobiose, and raffinose. Therefore, in some aspects, the sugar is selected from the group consisting of arabinose, xylose, cellobiose, and raffinose. The inventors presently disclose at least engineered proteins and methods of engineering microbial organisms and cells that allow for growth of microbial organisms and cells on non-native substrates that excludes at least one of glucose and sucrose. Therefore, in some aspects, the sugar excludes at least one of glucose and sucrose.

In another aspect, the present disclosure provides a method of engineering a microbial organism for growth on a non-native substrate, wherein the method comprises expressing an engineered protein in the microbial organism, wherein the engineered protein is Gal3p^{MC}, wherein the microbial organism is Saccharomyces cerevisiae, and wherein the non-native substrate is selected from the group consisting of arabinose, xylose, cellobiose, and raffinose. In some aspects, the method further comprises synergizing activation of the semi-synthetic GAL regulon with an optimized upstream heterologous metabolic module, and, in some aspects, the optimized upstream heterologous metabolic module comprises a deletion of GRE3 and/or an overexpression of at least one positive effector gene selected from the group consisting of TAL1, GAL2, araBAD, and XYLA*3-XKS1.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1: Diverse Synthetic Heterotrophy Using a Metabolic Coordinator

Engineering the utilization of non-native substrates, or synthetic heterotrophy, in proven industrial microbes such as Saccharomyces cerevisiae represents an opportunity to valorize plentiful and renewable sources of carbon and energy as inputs to bioprocesses. We previously demonstrated that activation of the galactose (GAL) regulon, a regulatory structure used by this yeast to coordinate substrate utilization with biomass formation during growth on galactose, during growth on the non-native substrate xylose results in a vastly altered gene expression profile and faster growth compared with constitutive overexpression of the same heterologous catabolic pathway. However, this effort involved the creation of a xylose-inducible variant of Gal3p (Gal3p^{Syn4.1}), the sensor protein of the GAL regulon, preventing this semi-synthetic regulon approach from being easily adapted to additional non-native substrates. Here, we report the construction of a variant Gal3p^{MC} (metabolic coordinator) that exhibits robust GAL regulon activation in the presence of structurally diverse substrates and recapitulates the dynamics of the native system. Multiple molecular modeling studies suggest that Gal3p^{MC} occupies conformational states corresponding to galactose-bound Gal3p in an inducer-independent manner. Using Gal3p^{MC} to test a regulon approach to the assimilation of the non-native lignocellulosic sugars xylose, arabinose, and cellobiose yields higher growth rates and final cell densities when compared with a constitutive overexpression of the same set of catabolic genes. The subsequent demonstration of rapid and complete co-utilization of all three non-native substrates suggests that Gal3p^{MC}-mediated dynamic global gene expression changes by GAL regulon activation may be universally beneficial for engineering synthetic heterotrophy.

Despite decades of effort, there is still a poor understanding of why synthetic heterotrophy is so recalcitrant in yeast. Our results indicate that if cells are “tricked” into thinking the non-native substrate is a native substrate, they are potentiated for rapid growth whereas, in the absence of this signal, cells suppress growth-promoting systems. Further, through our analysis, we find that the major limitation to substrate utilization is extrinsic-genes that control the flux of the substrate into central carbon metabolism—and not any downstream (intrinsic) yeast-specific factor. This is significant since the current paradigm is that the limitations are largely intrinsic. Finally, we show that by perturbing intrinsic factors, cells become less robust and sensitive to stressors. We conclude that much of the traditional engineering results in over-engineered strains with poor growth and robustness. Our proposed minimalistic, holistic engineering approach is more rapid, easier to optimize, and results in strains that maximize growth and robustness phenotypes.

### Introduction

The adoption of abundant and renewable substrates as inputs for biotechnology will be essential for creating a sustainable, circular bioeconomy (Langholtz et al., 2016; Rogers et al., 2017). But the inability of industrially important microbes, like Saccharomyces cerevisiae (“yeast”), to utilize many potential substrates poses a major hurdle to realizing this goal. The existing paradigm for engineering the assimilation of non-native substrates (i.e., synthetic heterotrophy) in yeast begins with the identification and constitutive overexpression of catabolic genes that enable the substrate to enter central carbon metabolism (CCM) where it is expected to be transformed into the key primary metabolites used for growth and biosynthesis. However, this method ignores the tight regulation of CCM and how these resources are distributed to accomplish cellular objectives (Nielsen and Keasling, 2016). The consequences of this oversight are reflected in the need for subsequent interventions, including flux balancing (Latimer et al., 2014; Kobayashi et al., 2018; Kim et al., 2013a), functional genomics (Ni et al., 2007; Mukherjee et al., 2021; Unrean et al., 2018; HamediRad et al., 2018; Chen et al., 2016), and adaptive lab evolution (Ha et al., 2011; Zhou et al., 2012; Sanchez et al., 2010), undertaken to improve growth and/or substrate utilization rate by resolving conflicts with cellular processes. Moreover, existing efforts are often focused on a specific substrate or small set of substrates—a siloed approach that makes it hard to translate findings to other substrates and even disincentivizes holistic thinking about the limits of metabolic plasticity/adaptability in this yeast. This has, at least in part, motivated domestication of other yeasts with broader substrate ranges (e.g., Scheffersomyces stipitis, Kluyveromyces marxianus) for biomanufacturing applications.

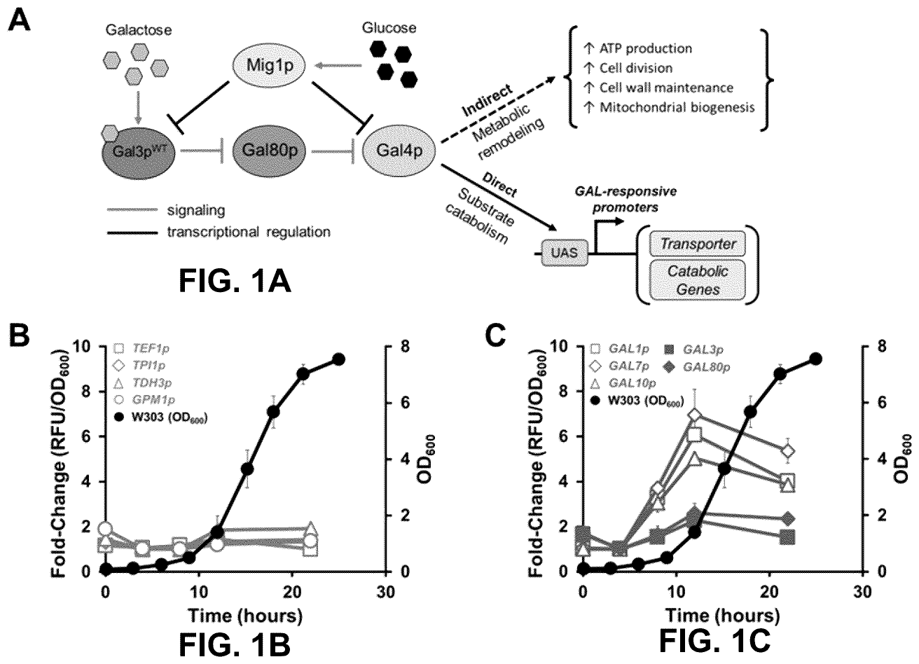

An alternate approach that we advocate here, is that instead of combating natural regulation we should leverage existing regulatory structures that have evolved to coordinate complex phenotypes like substrate utilization with biomass formation and metabolite synthesis. One such system is the galactose (GAL) regulon that yeast uses to co-ordinate substrate catabolism with global metabolism during growth on the native substrate galactose. In this system, the interaction of galactose with sensor protein Gal3p enables it to relieve the repression of Gal80p on the transcription factor Gal4p via protein-protein interactions (Lavy et al., 2012). Once freed from repression, Gal4p—the master activator of the GAL regulon—binds to its cognate Upstream Activating Sequences (UAS) to directly induce transcription of galactose catabolic genes (Leloir pathway) and indirectly modulates the transcript and/or protein abundance of numerous growth-associated genes (FIG. 1A) (Griffin et al., 2002; Ren et al., 1979). We previously reported the construction of a variant of sensor protein Gal3p (Gal3p^{Syn4.1}) that strongly activates the GAL regulon by the pentose sugars xylose and arabinose (Endalur Gopinarayanan and Nair, 2018; Trivedi et al., 2022). By placing heterologous genes for either xylose or arabinose catabolism under the control of GAL-responsive (Leloir pathway) promoters, the GAL regulon could be adapted for growth on these non-native substrates. Upon comparing this regulon-coordinated (REG) approach to growth with simple constitutive overexpression of the same catabolic genes, which we term a constitutive (CONS) approach, we found that our semi-synthetic regulon yielded superior growth rates on both xylose (0.24 h 1 vs. 0.11 h 1) and arabinose (0.27 h 1 vs. 0.06 h 1), respectively with minimal metabolic engineering (Trivedi et al., 2022). Significantly, a transcriptomic comparison showed relative upregulation of genes associated with cell division and mitochondrial biogenesis in the REG strain while the CONS strain showed upregulation of stress response and starvation-associated genes (Endalur Gopinarayanan and Nair, 2018). This suggests that the GAL regulon dynamically reshapes the cellular response for growth through direct and/or indirect action of Gal3p-Gal80p-Gal4p to potentiate the cells for rapid growth (Ren et al., 1979; Reimand et al., 2010), irrespective of the identity of the available substrate.

In this study, we demonstrate that, in addition to the known genome-wide changes in gene expression that occur upon GAL regulon activation, the expression from GAL-inducible promoters is dynamic and appears to be coordinated with growth whereas expression from constitutive promoters is constant. Hypothesizing that both aspects of this metabolic coordination may be universally beneficial for the assimilation of non-native substrates, we sought to develop this semi-synthetic regulon system into a platform approach that enables the rapid engineering of efficient synthetic heterotrophy without having to continually re-engineer substrate-specific induction. To do so, we created a variant of sensor protein that we term Gal3pMC (i.e., metabolic coordinator) and demonstrate that it, unlike the wild-type Gal3p (Gal3pWT; SEQ ID NO: 2), can strongly activate the GAL regulon on numerous structurally diverse substrates. We show that Gal3pMC can recapitulate the dynamic activation of the native regulon and, through the use of molecular dynamics and metadynamics simulations, that the mutations it carries enable it to do so in an inducer-independent manner. We found that this substrate-agnostic system retains the benefits of substrate-specific activation without any undue burden when engineering synthetic heterotrophy. We also show that using Gal3pMC to implement a REG approach to growth on three non-native substrates—xylose, arabinose, and cellobiose—yields superior performance when compared to a CONS (constitutive overexpression) approach. Finally, we provide a first demonstration that a single strain expressing Gal3pMC is capable of rapid, simultaneous, and complete utilization of all three non-native substrates concurrent with rapid growth—paving the way for a universal synthetic heterotrophy platform.

### Results

### Expression Under the GAL Regulon is Dynamic and Recapitulates Growth Phase

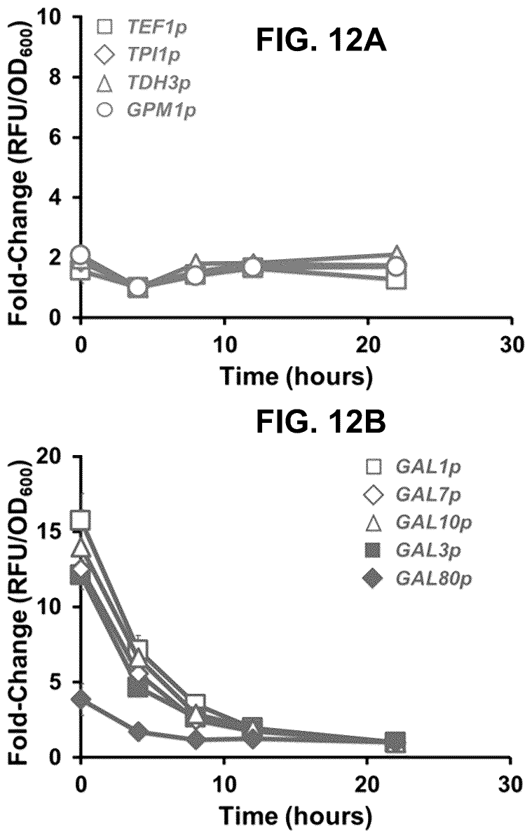

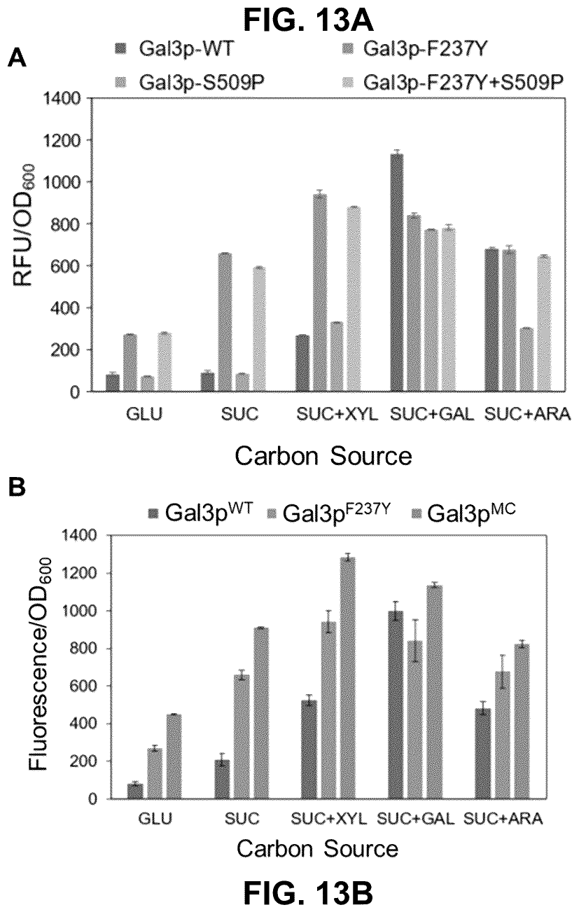

Existing efforts to engineer synthetic heterotrophy generally utilize strong, constitutive promoters to control the expression of the necessary catabolic genes. Constitutive promoters are natively able to recruit cellular transcriptional machinery to yield a roughly consistent expression level independent of cellular state and environmental context. This ‘always on’ phenotype contrasts with the control of catabolic gene expression observed in many regulatory structures that coordinate native substrate utilization in response to nutrient availability, including the GAL regulon of S. cerevisiae. To understand how these different approaches could impact the efficiency of substrate utilization, we used a fluorescent reporter (EGFP) to quantify the expression dynamics of several GAL-responsive (GAL1p, GAL7p, GAL10p, GAL3p, and GAL80p) and constitutive (TPI1p, TEF1p, TDH3p, and GPM1p) yeast promoters in a wild-type strain (W303-1a) that contains the native GAL regulon. During growth on galactose, we found that expression from the constitutive promoters was relatively stable across a 24 h period (varying less than two-fold) (FIG. 1B). In contrast, expression from GAL-responsive promoters starts lower (˜50% of constitutive promoters) but increases by ˜4-6 fold for the promoters that control the expression of the galactose catabolic (Leloir pathway) genes (GAL1p, GAL7p, and GAL10p) while remaining relatively constant for the promoters controlling regulatory genes (GAL3p and GAL80p) (FIG. 1C). When we overlaid the OD₆₀₀ profile, we observed that expression from Leloir pathway promoters tracks with growth phase, with the lowest expression levels occurring when the cells are in lag phase (t 0 h), maximum expression during the exponential growth phase, and finally diminishing expression as galactose is depleted and the cells enter stationary phase (t >20 h). During growth on glucose, the expression profile of the constitutive promoters was largely unchanged (FIG. 12A) while expression from GAL-inducible promoters is strongly repressed because of carbon catabolite repression (FIG. 13B). Overall, GAL-responsive promoters are more dynamic and stronger (upon full activation) relative to constitutive promoters (Peng et al., 2015; Deng et al., 2021).

### Gal3p^{MC} Activates the GAL Regulon in the Presence of Structurally Diverse Carbon Sources

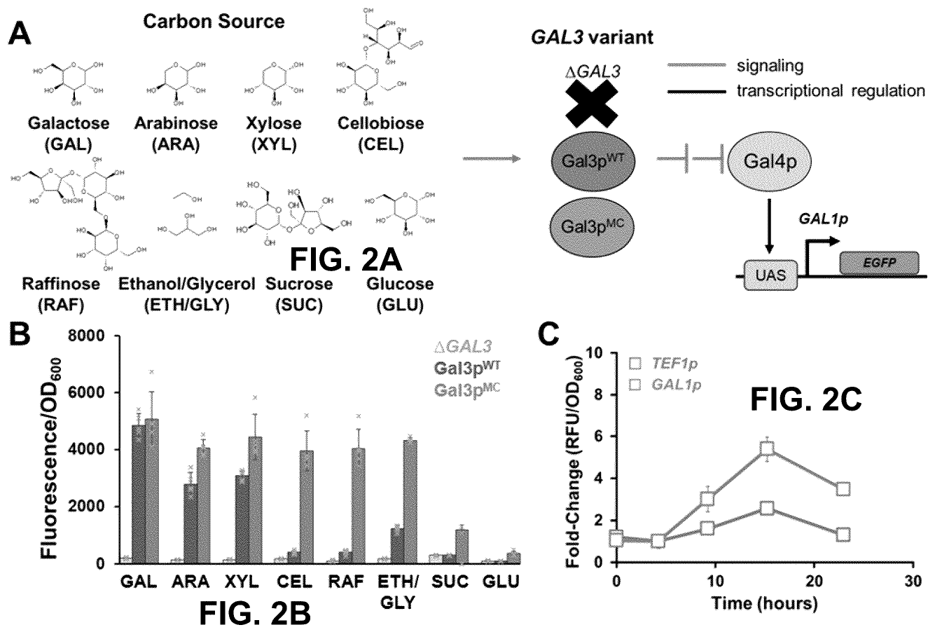

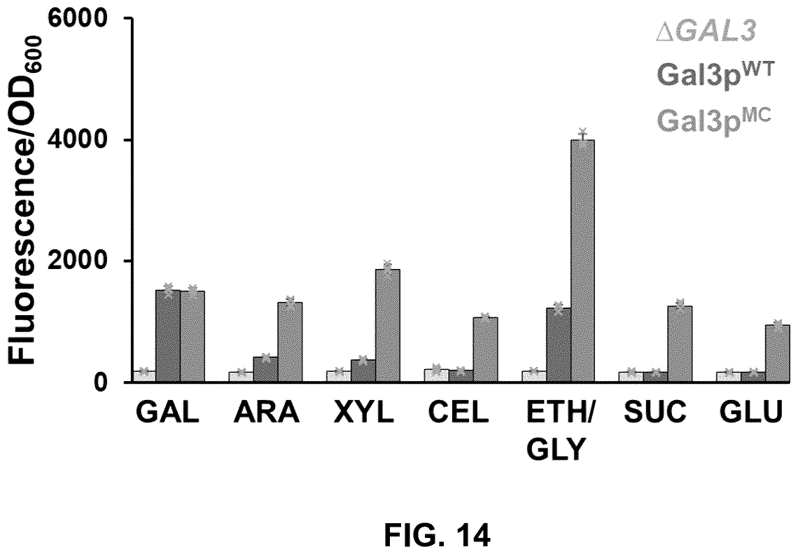

While the creation of a xylose-inducible sensor protein variant Gal3p^{Syn4.1} enabled us to adapt the GAL regulon for growth on xylose (Endalur Gopinarayanan and Nair, 2018), we wanted to develop a method for rapidly applying a regulon approach to other non-native substrates without having to repeatedly re-engineer substrate-specific sensing. Blank et al. previously reported various autoactivating Gal3p variants but we found that the best among those (viz. F237Y, S509P) could only partially activate GAL promoters without galactose (FIG. 13A) (Blank et al., 1997). To enable complete activation, equivalent to the WT GAL regulon with galactose, we combined the F237Y mutation to our Gal3p^{Syn4.1} variant (Gopinarayanan and Nair, 2018) to yield a mutant we term Gal3pMC (metabolic coordinator) (FIG. 13B). To compare the ability of Gal3pWT and Gal3pMC to activate the regulon in the presence of a given carbon source, we monitored the fluorescence resulting from a GAL1p-yEGFP reporter construct (FIG. 2A) in the presence of numerous structurally distinct carbon sources substrates—galactose, arabinose, xylose, cellobiose, raffinose, ethanol/glycerol, sucrose, and glucose—in a strain (VEG16) that lacks the galactose sensing and catabolic genes (ΔGAL3; ΔGAL1; ΔGAL10; ΔGAL7; ΔGRE3) but retains GAL80 and GAL4. We performed this experiment using two native carbon sources, raffinose (FIG. 2B) or sucrose (FIG. 14), to support cell growth. We observed that Gal3pWT strongly activates the regulon in the presence of its native inducer galactose and to a lesser extent xylose and arabinose, likely due to the structural similarity between these substrates. The indistinguishable signal between Gal3pWT and the ΔGAL3 control in presence of glucose reflects carbon catabolite repression while the small increase in fluorescence on raffinose and cellobiose likely reflects the weak activation known to result from overexpression of Gal3pWT in the absence of glucose (Bhat and Hopper, 1992).

In contrast, Gal3pMC activates the GAL regulon in the presence of all the non-native substrates to a similar extent as Gal3pWT on galactose while remaining repressible by glucose and sucrose (FIG. 2B), a result that suggests Gal3pMC may be capable of recapitulating the dynamic catabolic gene expression profile and genome-wide expression changes known to support optimal growth on the native substrate galactose during growth on non-native substrates (Malakar and Venkatesh, 2014). To determine if regulon activation using Gal3pMC remains dynamic, we constructed a strain (SFS6) that contains copies of GAL3MC integrated into the chromosome under the control of both GAL1p and GAL3p. This dual-feedback loop configuration mimics the organization of the native GAL regulon and has been demonstrated to increase both the overall magnitude and the homogeneity of activation to better effect a switch-like behavior among cells in the population (Endalur Gopinar-ayanan and Nair, 2018; Venturelli et al., 2012). As SFS6 lacks the Leloir pathway (ΔGAL1 7110), these genes were supplied under the control of their native promoters on a plasmid (pRS423-GAL-REG) to facilitate growth on galactose. Using strain SFS6, we assessed the expression profile of the constitutive TEF1p-EGFP and GAL-responsive GAL1p-EGFP constructs during growth on galactose (FIG. 2C). While expression from TEF1p was observed to increase by 2-3-fold at one time point, it generally stayed within a two-fold range whereas expression from GAL1p appeared to mimic the profile observed upon activation by the wild-type regulon (FIG. 1C), exhibiting a >5-fold increase in fluorescence.

### Molecular Simulations Support Inducer-Independent Regulon Activation by Gal3pMC

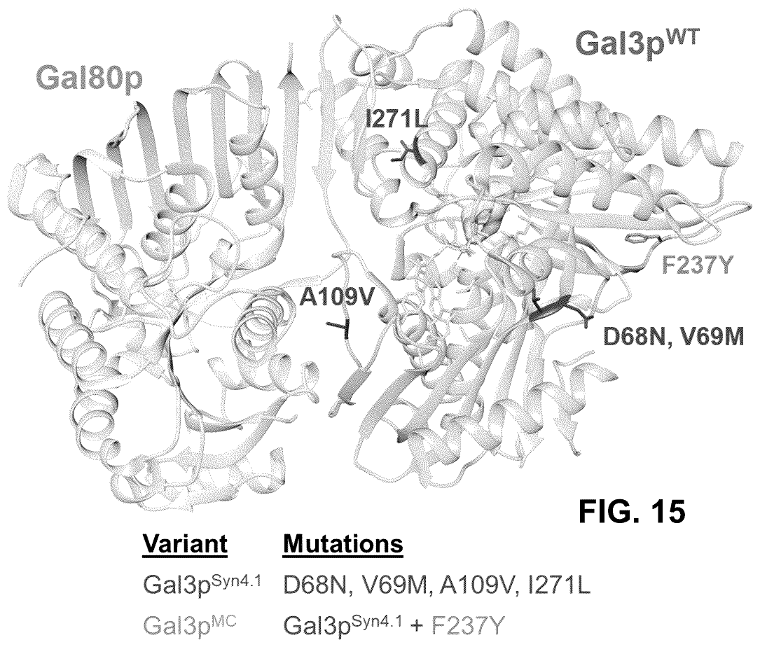

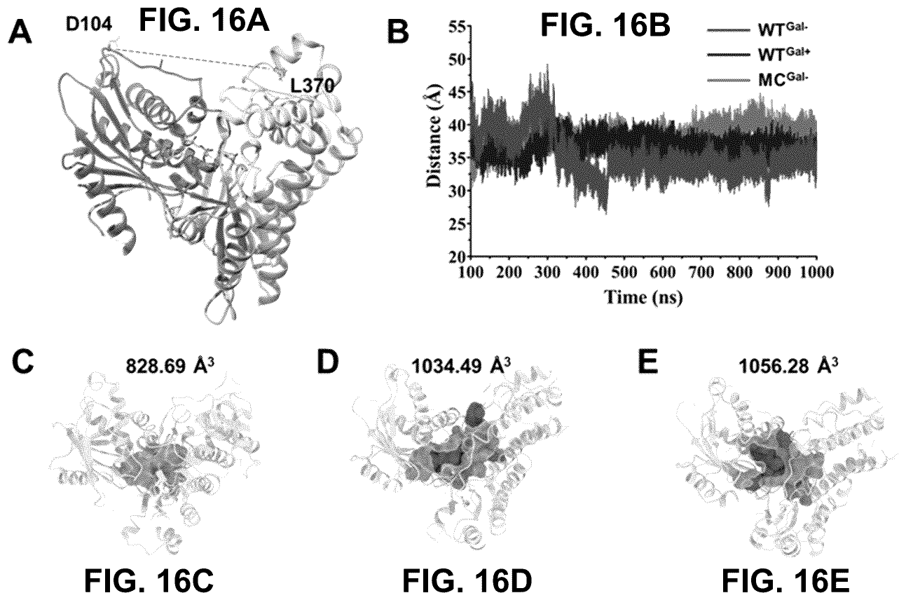

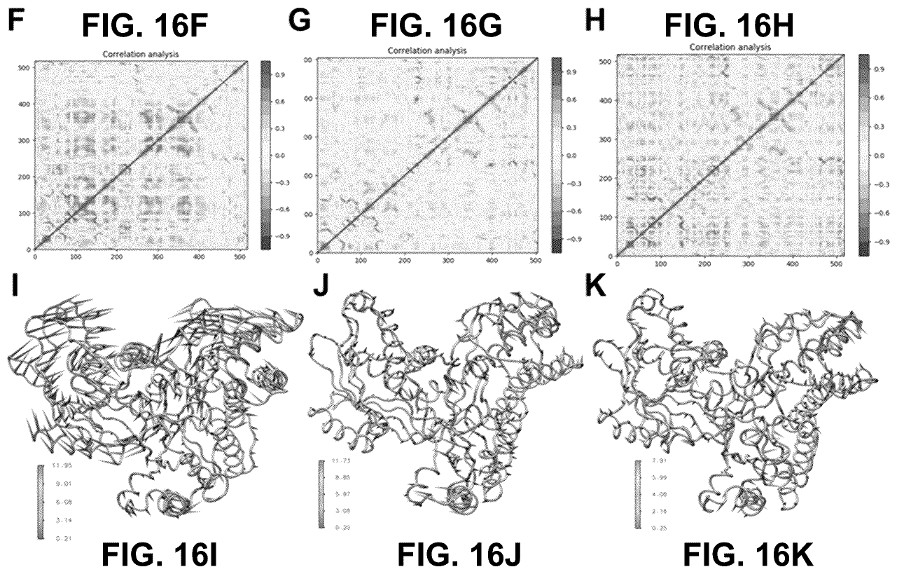

Experiments have shown that the binding of Gal3pWT to its ligand galactose induces and stabilizes a conformational change from an ‘open’ to a ‘closed’ state that facilitates interaction with Gal80p and subsequent de-repression of Gal4p (Lavy et al., 2012). However, mapping the mutations carried by Gal3pMC onto a structure of Gal3pWT in complex with Gal80p (PDB: 3V2U) reveals that the mutations are distributed throughout the protein (FIG. 15). Combined with the structural diversity of substrates (pentoses, hexose, disaccharide, and trisaccharide) on which Gal3pMC exhibits robust regulon activation, observations suggest that Gal3pMC may adopt a ‘closed’ conformation that enables it to bind Gal80p and relieve repression on Gal4p without requiring interaction with a ligand/inducer. As the transition between open and closed states is associated with the relative movement of two domains in Gal3pWT, while a third ‘hinge’ region remains relatively static, we decided to explore this inducer-independent activation hypothesis using molecular dynamics (MD) simulations. To do so, we monitored the distance be-tween two residues (D104 and L370) found on opposing moving domains (FIG. 16A) over the course of 1000 ns molecular dynamics (MD) simulations of Gal3pWT in complex with ATP and Mg2+ without galactose (WTGal−), Gal3pWT in complex with ATP, Mg2+, and galactose (WTGal+), and Gal3pMC in complex ATP and Mg2+ without galactose (MCGal−). Simulations revealed that distances between the residues were more similar between MCGal− and WTGal+(FIG. 16B). Calculations of active site cavities at the end of the 1000 ns simulation also revealed a similar trend (FIG. 16C-E). To understand which residues most contributed to the dynamics observed in each of the three systems during these simulations, we performed cross-correlational studies of the relative movement between the residues of WTGal−, WTGal+, and MCGal− (FIG. 16F-H). The lower intensities of residue-residue correlation in WTGal+ and MCGal− maps indicate relatively stable dynamics relative to those of WTGal−. To visualize the domain movements in detail, we used principal component analysis (PCA) for all three systems across the 1000 ns MD trajectories. Porcupine plots generated based on the extreme projections of the PCA show the intensity and direction of the motion of the C atoms in each structure. WTGal− showed highly dynamic conformational states while MCGal− and WTGal+ showed relatively limited dynamics across the structure (FIG. 16I-K).

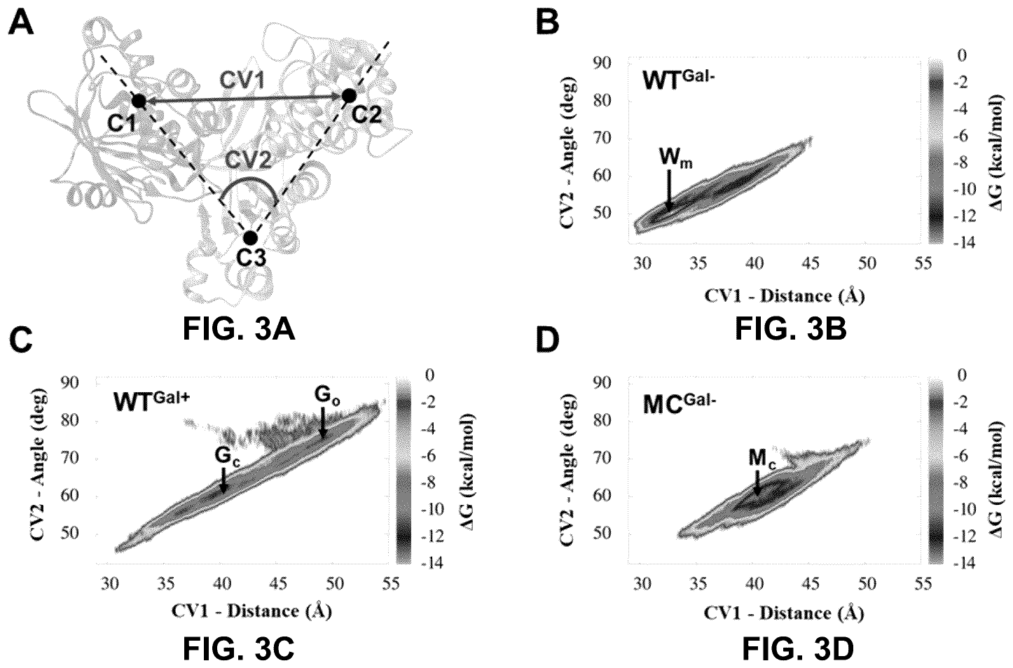

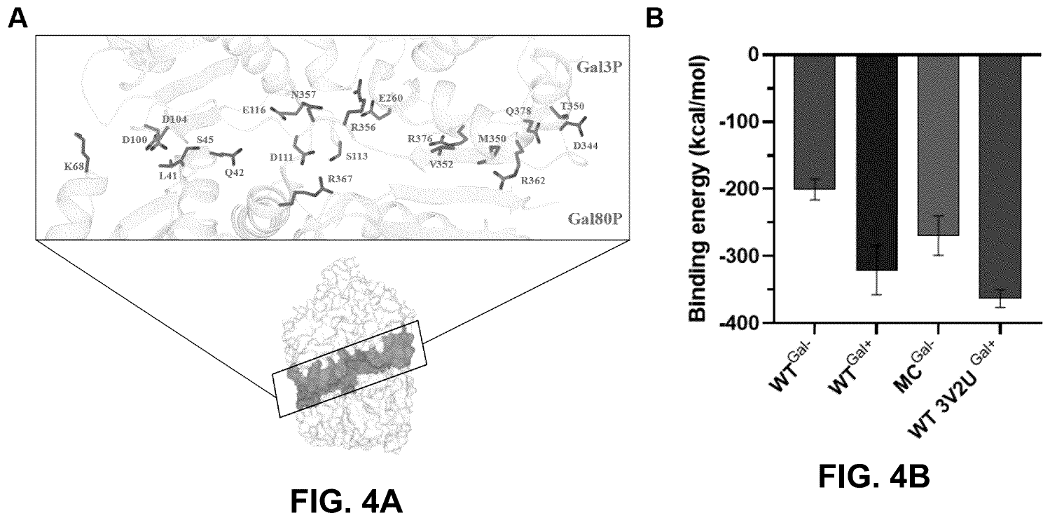

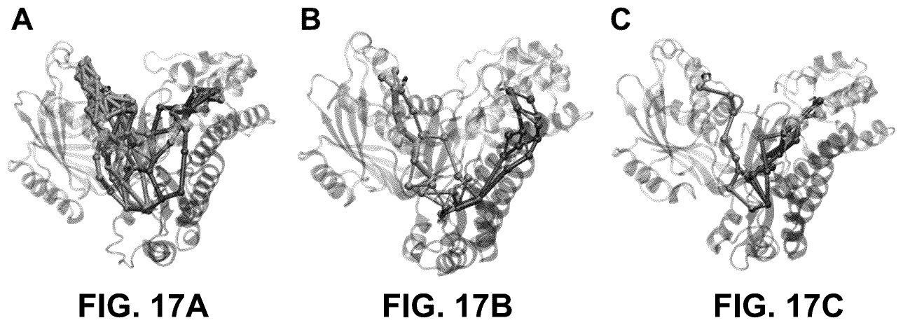

Initializing the molecular dynamics simulations of the three conditions in the ‘closed’ conformation introduces a bias that prevents each system from adopting a range of conformational states. Metadynamics-based simulations use a potential bias to move a system out of a local energy minimum enabling the calculation of the free energy surface (FES) across a range of conformations. To perform these complementary metadynamics analyses, we began to define the conformational landscape to be explored by identifying centers of mass (COM) for each of the three Gal3p domains (C1-C3). Next, we defined the two collective variables (CV) to be manipulated during the simulation as (i) the distance between the COM of the two moving domains (C1 and C2) and (ii) the angle formed by the three COM at the hinge region (C3) (FIG. 3A). Examining the resulting FES for each system reveals differences in the CV values that yield energetically favorable structural conformations. The FES of WTGal− (FIG. 3B) showed two Gaussian wells, one for a short-lived partially open state and one for an exceptionally prominent minimum corresponding to an irreversibly collapsed closed conformation catalyzed by strong interaction networks between the two moving domains (FIG. 17A) not observed between the moving domains in the WTGal+(FIG. 17B) and MCGal− (FIG. 17C) systems. Therefore, it can be called a misfolded state (Wₘ) of the protein as the recovery of an open conformation would be highly energy-consuming, and therefore, unlikely. The FES of WTGal+ showed a path of conformational transitions that leads from a closed (G_{c}), but not collapsed, state to a fully open state (Gₒ) of the protein (FIG. 3C). Significantly, the closed state of WTGal+(G_{c}) is structurally distinct from the closed, collapsed/misfolded state (Wₘ) adopted by WTGal− suggesting that the latter is unlikely to interact with Gal80p in a productive manner. As the metadynamics simulation progresses, WTGal+ eventually adopts an open state (Gₒ) that visualization studies reveal to be a consequence of substate egress. The FES of MCGal− (FIG. 3D) showed a single predominant minimum corresponding to a closed state (M_{c}) that closely resembles the closed state (G_{c}) of WTGal+. These results suggest that the mutant Gal3pMC forms a stable closed conformation closely resembling the closed WTGal+ conformation, with a similar interaction network observed between the moving domains. When combined with the previously observed robust activation on structurally diverse substrates, the similarity between the closed state conformations adopted by WTGal+ and the MCGal− systems suggests that Gal3pMC can bind and sequester Gal80p in an inducer-independent manner. Interactions between Gal3pMC with Gal80p are energetically favorable We compared the energetics of Gal3p-Gal80p binding using the minimum energy conformations (Wₘ, G_{c}, M_{c}) obtained from metadynamics-based simulations. Docking simulations between Gal3p and Gal80p were run for 50 ns and binding energy was calculated every 0.02 ns (FIG. 4A). Among the complexes formed, the WTGal+ Gal80p was energetically the most like the crystal structure complex (FIG. 4B). The results also revealed that the complex formed by WTGal− with Gal80p was significantly less energetically favorable than the complexes formed by WTGal+ and MCGal−. This data further supports the idea that Gal3pMC may be able to activate the GAL regulon in a substrate independent manner. Recognizing that substrate-agnostic activation phenotype of Gal3pMC represents a departure from the inducer-dependent regulatory logic of the native regulon, we decided to test the fitness consequences of this transition when utilizing a regulon approach to engineer synthetic heterotrophy.

### Substrate-Agnostic and Substrate-Specific GAL Regulon Activation are Equivalent

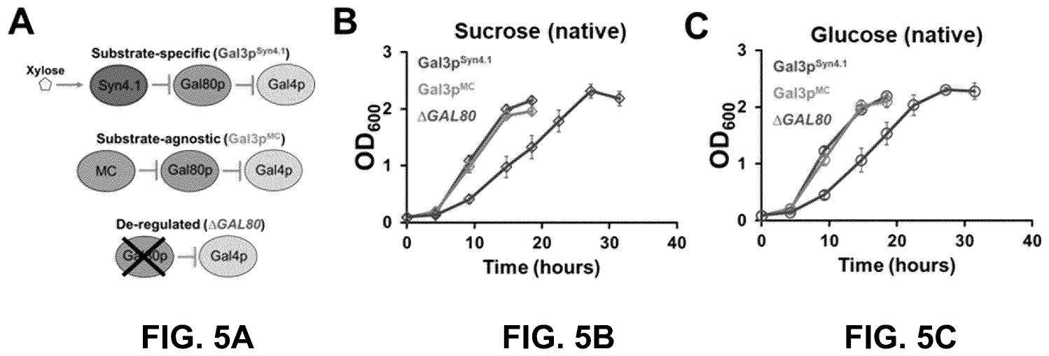

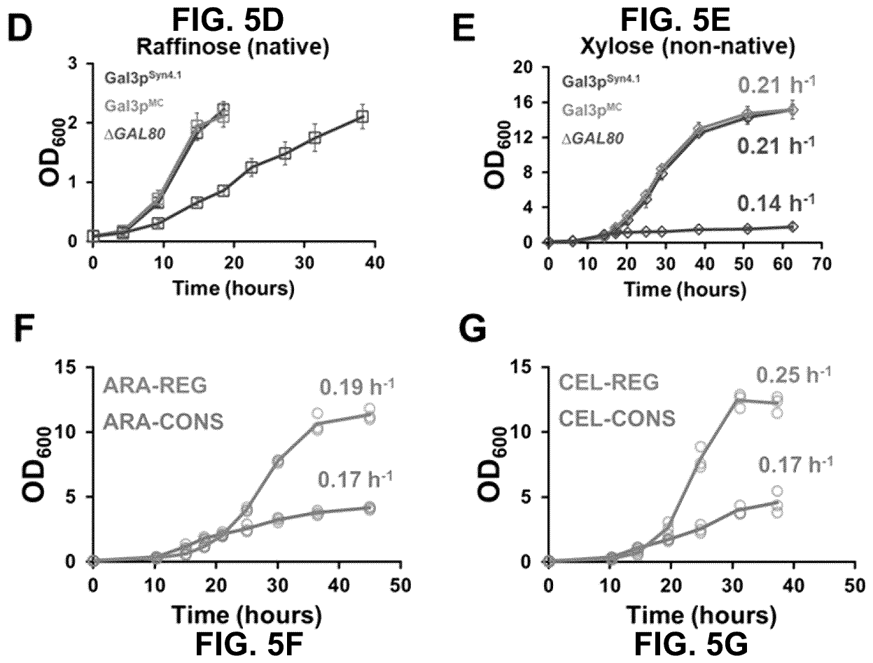

During our previous effort to adapt the GAL regulon to growth on xylose, regulon activation was accomplished using the xylose-inducible sensor-protein variant Gal3pSyn4.1 18. To compare the performance of inducer-dependent (substrate-specific) and inducer-independent (substrate-agnostic) approaches for engineering synthetic heterotrophy, we transformed a plasmid (pRS426-XYL-REG) encoding the isomerase pathway genes for xylose utilization (XYLA*3 (Lee et al., 2012), XKSI) under the control of GAL-responsive promoters into strains engineered for (i) xylose-inducible regulon activation using Gal3pSyn4.1 (strain VDT13), (ii) substrate-agnostic regulon activation using Gal3pMC (strain SFS6), and (iii) substrate-agnostic activation via deletion of GAL80, the master repressor of the GAL regulon (strain SFS11), as disruption of this regulatory gene has been demonstrated to yield constitutive expression of galactose catabolic genes and enhance galactose utilization (Quar-terman et al., 2016; Torchia et al., 1984) (FIG. 5A). While regulon activation in both SFS6 and VDT13 requires an interaction between Gal80p and their respective Gal3p variants (Gal3pMC and Gal3pSyn4.1), activation in SFS11 is totally de-regulated due to the absence of Gal80p.

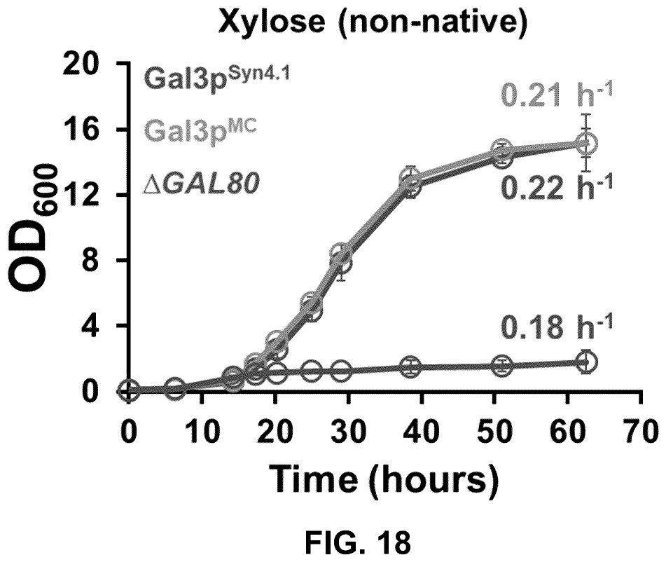

In addition, all three strains contain two accessory genes found to improve pentose utilization—the transporter GAL22.1 (Reznicek et al., 2015) and the transaldolase TAL1—integrated into the chromosome under the control of GAL-responsive promoters. The growth of these three strains in defined (SC) media containing the native carbon sources sucrose (FIG. 5B), glucose (FIG. 5C), or raffinose (FIG. 5D) was monitored to identify the potential fitness defects of each approach during strain handling. While all three approaches reached similar final cell concentration on each of the three substrates, the ΔGAL80 condition took roughly twice the time to do so compared with Gal3pSyn4.1 and Gal3pMC (˜30 h vs. ˜18 h) suggesting that de-regulation of the regulon results in reduced fitness relative to strains in which the native regulatory organization is maintained. As sucrose and glucose are both capable of exerting repression on the GAL regulon (Trivedi et al., 2022), sub-culturing all three strains from these two native substrates into complex (YP) media containing the non-native substrate xylose should therefore result in growth-coupled expression of xylose catabolic genes and rapid growth. Upon subculturing from sucrose, we observed that all three approaches enabled growth. However, the lower growth rate seen for the ΔGAL80 strain compared with cells carrying GAL3Syn4.1 or GAL3MC (μ=0.14±0.001 h 1, 0.21±0.008 h 1, and 0.21±0.001 h 1, respectively) combined with the drastically lower final cell density supports maintenance of native regulatory structure over abolished control (FIG. 5E). Subculturing from glucose as an overnight carbon source yielded similar results (FIG. 18). Significantly, the equivalent performance of GAL3Syn4.1 and GAL3MC backgrounds on both native and non-native carbon sources indicates that inducer-independent activation enables the metabolic coordination of a regulon approach to be applied for engineering synthetic heterotrophy in a substrate-agnostic manner provided that the native regulatory organization is maintained.

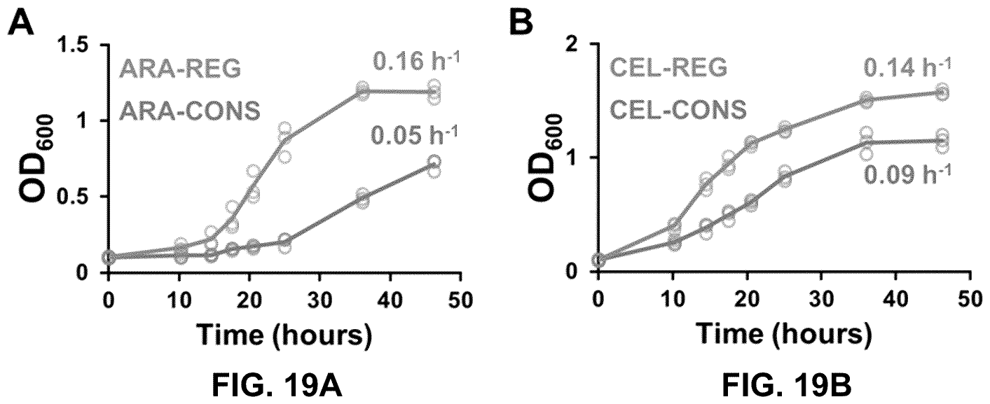

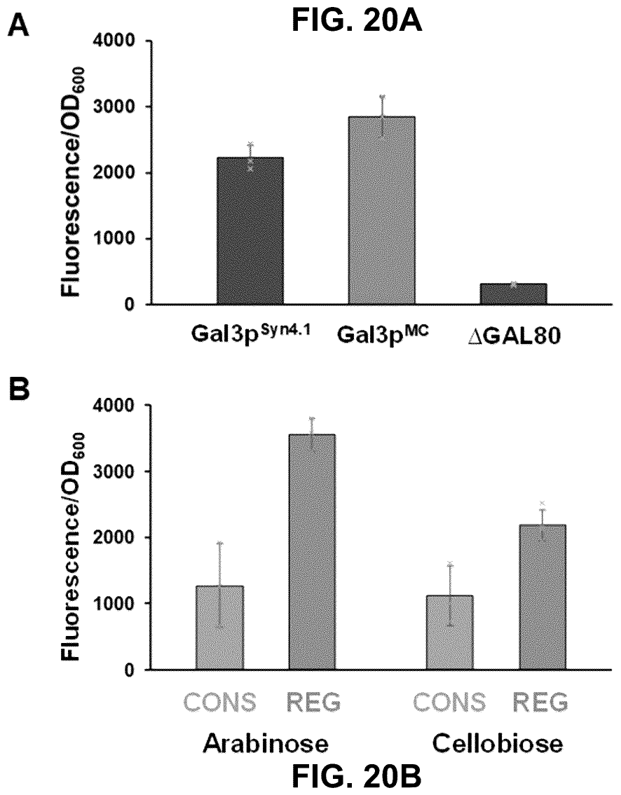

To confirm that these results were not xylose-specific, we proceeded to use Gal3pMC to compare REG and CONS approaches to growth on two additional non-native, lignocellulosic substrates—cellobiose and arabinose. To do so, we placed catabolic genes previously identified to enable the efficient utilization of arabinose (araA, araB, and araD from Lacto-bacillus plantarum (Wisselink et al., 2007)) and cellobiose (cdt-1 and ghl-1 from Neurospora crassa (Galazka et al., 1979)) under the control of the GAL-responsive promoters GAL1p, GAL10p, and GAL7p and the strong, constitutive promoters TEF1p, TPI1p, and GPM1p to yield plasmids pRS426-ARA-REG, pRS426-ARA-CONS, pRS426-CEL-REG, and pRS426-CEL-CONS, respectively. Transforming pRS426-ARA-REG into GAL3MC integrant strain SFS6 (containing GAL-inducible copies of GAL22.1 and TAL1) yielded strain ARA-REG while transforming pRS426-ARA-CONS into strain VDT27 (that contains GAL22.1 and TAL1 under the control of strong constitutive promoters) yielded strain ARA-CONS. Similarly, transforming pRS426-CEL-REG and pRS426-CEL-CONS into strains SFS3 and VEG16, respectively, that are comparable to SFS6 and VDT27 except that they lack accessory genes GAL22.1 and TAL1, yielded strains CEL-REG and CEL-CONS. The REG strain exhibited a higher growth rate than the CONS strain on both defined (0.16±0.005 h 1 vs. 0.05±0.005 h 1) (FIG. 19A) and complex (0.19±0.002 h 1 vs. 0.17±0.003 h 1) (FIG. 5F) media containing 2% arabinose as a sole carbon source. Similarly, the REG strain grew faster than its CONS counterpart on both defined (0.14 0.001 h 1 vs. 0.09 0.005 h 1) (FIG. 19B) and complex (0.25±0.002 h 1 vs. 0.17±0.003 h 1) (FIG. 5G) media containing 2% cellobiose as a sole carbon source. In general, we found that the strength of the promoters driving the catabolic gene expression corresponded well with growth phenotype (FIG. 20). Taken together, these data demonstrate that the metabolic coordination effected by Gal3pMC is broadly beneficial to growth on non-native carbon sources compared with the existing constitutive paradigm with minimal additional engineering.

### Gal3pMC Enables Rapid and Complete Co-Utilization of Multiple Non-Native Substrates

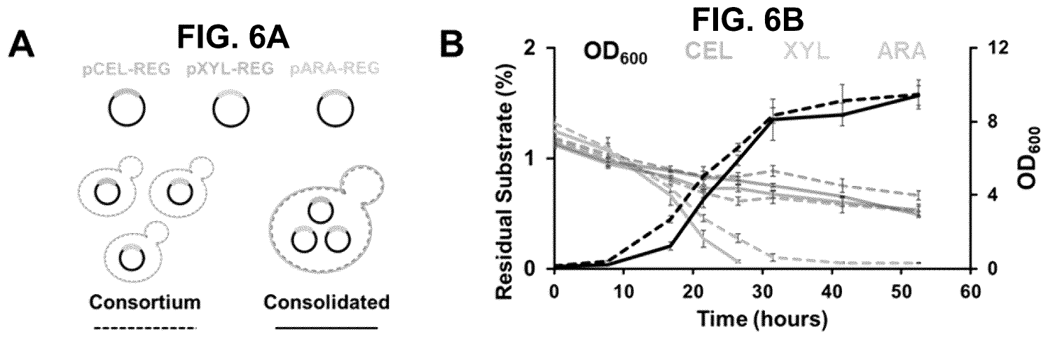

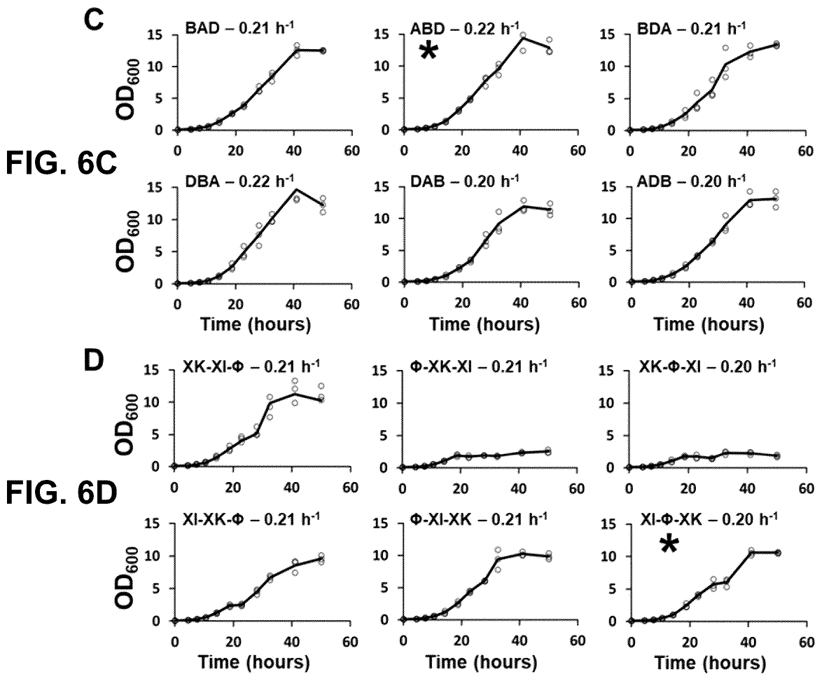

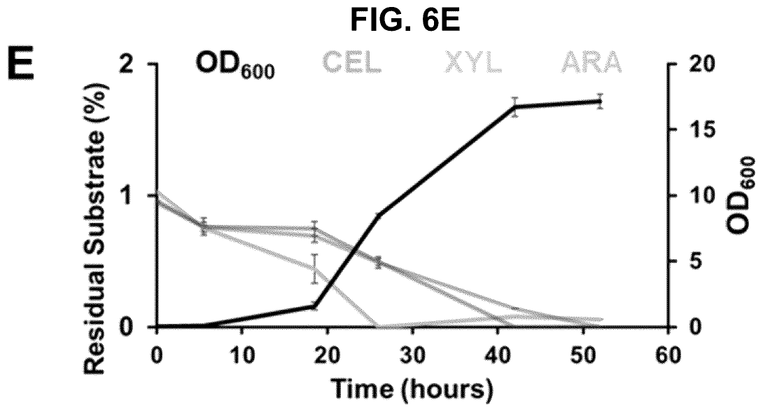

Having demonstrated that a regulon approach is beneficial to growth on all three non-native, lignocellulosic substrates individually, we sought to utilize the inducer-independent regulon activation of Gal3pMC to test a REG approach to their co-utilization. As previous studies have observed that the division of catabolic labor among a consortium of strains can enable faster and/or more efficient co-utilization when compared with a single strain engineered to utilize all substrates simultaneously (Eiteman et al., 2008; Verhoeven et al., 2018; Flores et al., 2020), we decided to test both approaches. To accommodate all three plasmids into a single strain, the catabolic genes for each substrate were moved to plasmids containing unique selectable markers to create pRS423-XYL-REG, pRS425-ARA-REG, and pRS424-CEL-REG. Trans-forming these plasmids into strains SFS6, SFS6, and SFS3, respectively, represented a three-strain (Consortium) approach, while transforming all three plasmids into SFS6 represented a single strain (Consolidated) approach (FIG. 6A). While both Consortium and Consolidated approaches exhibited simultaneous co-utilization of all carbon sources and achieved similar final cell densities during growth on YP media containing 1% of each substrate, we were surprised to find that the Consolidated approach resulted in a faster maximum growth rate than the Consortium approach (0.20±0.003 h 1 vs. 0.17±0.003 h 1; two-tailed, two-sample t-test, t 8.41, df 3, p<0.01) (FIG. 6B). However, neither approach resulted in complete pentose consumption even after cellobiose was fully depleted. This led us to hypothesize that there may be flux imbalances in the pentose utilization pathways.

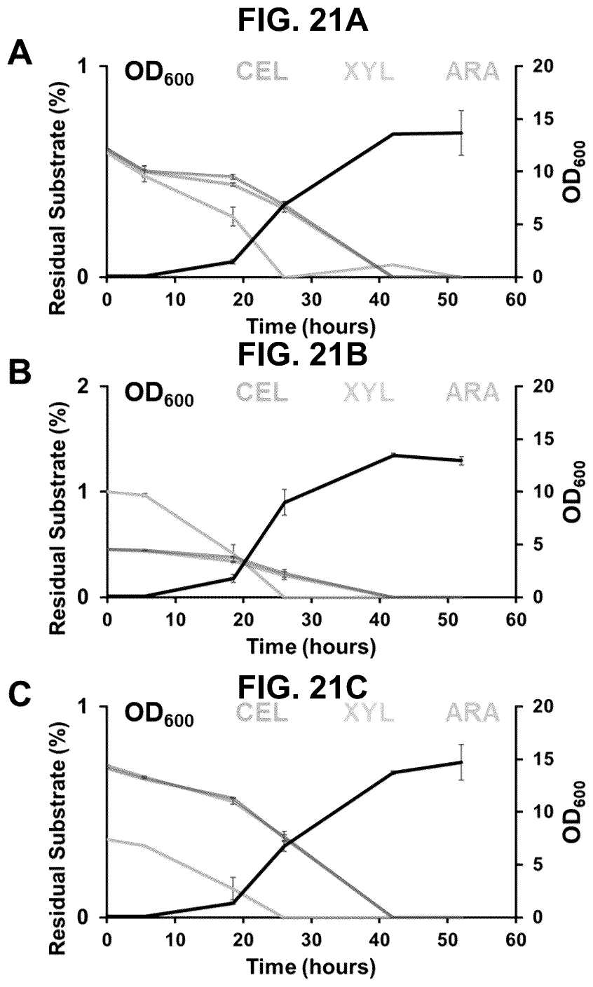

The success of combinatorial pathway design in optimizing both catabolic (Latimer et al., 2014; Kim et al., 2013b; Protzko et al., 2018) and anabolic pathways (Ajikumar et al., 1979; Smanski et al., 2014) motivated us to test the performance of each of the six possible GAL-responsive promoter-catabolic gene pairings for arabinose (FIG. 6C) and xylose (FIG. 6D) in Gal3pMC integrant strain SFS6. The arabinose constructs all performed similarly, with growth rates clustered in a small range of values (0.20 0.22 h 1). A similar range of growth rates was observed for the xylose constructs (0.20 0.21 h 1) but the substantially reduced final cell density of two of the arrangements (φ-XK-XI and XK-φ-XI) indicates that there can be fitness consequences to expression imbalances. As no clearly superior combination could be identified to the initial arabinose and xylose designs, we decided to re-test a Consolidated approach to co-utilization on 1% of each substrate (FIG. 6E) using designs (ABD for arabinose, XI-φ-XK for xylose) identified as optimal in a related investigation by our lab into efficient pentose assimilation (Trivedi et al., 2022). Hypothesizing that incomplete pentose utilization may also reflect limitations in the carrying capacity of the medium itself, we also tested different substrate ratios (cellobiose-xylose-arabinose: 0.7%-0.7%-0.7%, 1%-0.5%-0.5%, and 0.4%- 0.8%-0.8%) (FIG. 21A-C) at a lower (˜2%) total substrate concentration. Encouragingly, all substrates were utilized to undetectable levels (<0.05%, limit of detection) in approximately 50 h of cultivation for all conditions. For the 3% total sugar condition, we observed an 81% improvement in final cell density when using the optimized pentose plasmids compared with the non-optimized (OD₆₀₀ 17.2 vs. 9.5, respectively). For the three 2% total substrate conditions, the final cell density was 41% greater on average using the same comparison (OD₆₀₀ ˜13.5 vs. 9.5, respectively). Despite their similar performance during growth on a single sugar, the higher final OD₆₀₀ values and complete substrate utilization observed upon switching from the initial to the optimized designs suggests that the relative expression level of catabolic genes optimal for co-utilization may be different than for the single substrate. Considering that the highest single-sugar growth rates obtained in this study are either the best (for cellobiose) or competitive with the best (xylose, arabinose) aerobic growth rates observed in the literature (Table 1) without the need for extensive genome editing or adaptive laboratory evolution (ALE), we believe that together these results demonstrate the flexibility and effectiveness of utilizing a regulon approach to engineer efficient synthetic heterotrophy on diverse substrates.

### Discussion

The existing paradigm for engineering synthetic heterotrophy consists of constitutive expression of catabolic genes that channel the carbon source into central carbon metabolism (CCM) and largely ignores cellular regulation. In contrast, the assimilation of native carbon sources is often controlled by regulatory structures called regulons that coordinate substrate catabolism with broader metabolism to optimize biomass formation. Activation of the galactose (GAL) regulon, for instance, results not only in the expression of the genes necessary for the catabolism of galactose but global metabolic remodeling to support efficient growth (Ren et al., 1979; Reimand et al., 2010). In previous work, we established that adapting the GAL regulon to growth on the non-native substrate xylose improved growth rate and substrate utilization when compared with a constitutive approach (Endalur Gopinarayanan and Nair, 2018). This surprising result motivated us to explore the potential of a semi-synthetic regulon system as a general approach to engineering synthetic heterotrophy in the important industrial microbe S. cerevisiae so that it can be easily and rapidly applied to other attractive potential substrates for yeast biotechnology.

TABLE 1
Aerobic growth rates of previously engineered S. cerevisiae strains
on xylose, arabinose, and cellobiose including relevant genotypes.

| Substrate | Strain | Relevant Genotype | μ (h⁻¹) | Reference |
|---|---|---|---|---|
| Xylose | X389 | PiXI, PsXK, HXT7m, PsTAL1, TKL1, | 0.26 | ¹ |
| | | RPE1, RKI1 ΔGRE3, ΔASK10, ΔYPR1 | | |
| | LI110A | SsXR, SsXDH*, SsXK, SsRPE, SsRKI, | 0.25 | ² |
| | | SsTKL, SsTAL; Evolved | | |
| | XYL-REG | xylA*3, XKS1, TAL1, GAL2^{2.1}, GAL3^{Syn4.1} | 0.24 | ³ |
| | (Syn4.1) | under GAL promoters, ΔGAL1/3/7/10, | | |
| | | ΔGRE3 | | |
| | YRH1114 | PrXK, PrXI, codons optimized for S. | 0.23 | ⁴ |
| | | cerevisiae; Evolved | | |
| | RWB 217 | XYLA, XKS1, TAL1, TKL1, RPE1, RKI1, | 0.22 | ⁵ |
| | | ΔGRE3; Evolved | | |
| | XYL-REG (MC) | xylA*3, XKS1, TAL1, GAL2^{2.1}, GAL3^{MC} | 0.21 | This Study |
| | | under GAL promoters, ΔGAL1/3/7/10, | | |
| | | ΔGRE3 | | |
| | H131-A3^{SB-1} | XYLA, PsXYL3, PsTAL1, TKL1, RPE1, | 0.197 | ⁶ |
| | | RKI1; Evolved | | |
| | CMB.GS010 | PsXYL1, PsXYL2, PsXYL3; Evolved | 0.18 | ⁷ |
| | SyBE005 | XYL1, mXYL2, XKS1, RPE1, TAL1, RKI1, | 0.165 | ⁸ |
| | | TKL1, ΔAUR1; Evolved | | |
| | UUU | UbXR1, UbXDH, UbXK | 0.15 | ⁹ |
| | ATCC-F2 | pgXR, pcXDH, aoXKS; Evolved | 0.15 | ¹⁰ |
| | ADAP8 | XYLA, XKS, SUT1; Evolved | 0.133 | ¹¹ |
| | SXA-R2P-E | Ss_xyla*3, XKS1, Ss_tal1, ΔGRE3, | 0.128 | ¹² |
| | | ΔPHO13; Evolved | | |
| | YSX3-TAL1M | XYL1, XYL2, PsXYL3, psTAL1 | 0.117 | ¹³ |
| Arabinose | ARA-REG | Lp_araA, Lp_araB, Lp_araD, TAL1, | 0.27 | ³ |
| | (Syn4.1) | GAL2-2.1, GAL3^{Syn4.} under GAL | | |
| | | promoters, ΔGAL1/3/7/10, ΔGRE3 | | |
| | IMX728 | araA, araB, araD, Pc_araT, ΔGLK1, | 0.26 | ¹⁴ |
| | | ΔHXK1, ΔHXK2, ΔGAL1, ΔGAL80, | | |
| | | ΔGRE3, RPE1, TKL1, TAL1, NQM1, | | |
| | | RKI1, TKL2 | | |
| | ARA-REG (MC) | Lp_araA, Lp_araB, Lp_araD, TAL1, | 0.22 | This Study |
| | | GAL2-2.1, GAL3^{MC} under GAL | | |
| | | promoters, ΔGAL1/3/7/10, ΔGRE3 | | |
| | IMS0002 | Piromyces xylA, Lp_araA, Lp_araB, | 0.15 | ¹⁵ |
| | | Lp_araD, XKS1, TAL1, TKL1, RPE1, | | |
| | | RKI1, ΔGRE3 | | |
| | BWY1-SI | araA (codon optimized), araBG361A, | 0.112 | ¹⁶ |
| | | araD, GAL2; Evolved | | |
| | TMB3664 | XKS1, TAL1, TKL1, RKI1, RPE1, | 0.05 | ¹⁷ |
| | | XYL1^{K270R}, XYL2, sLAD1 (synthetic), | | |
| | | sALX1(synthetic), ΔGRE3 | | |
| | TMB3076 | Bs_araA, Ec_araD, Ec_araB, XKS1, | 0.03 | ¹⁸ |
| | | TAL1, TKL1, RKI1, RPE1, ΔGRE3 | | |

In this work, we demonstrate that expression from the GAL-responsive promoters that natively control the expression of galactose catabolic genes (Leloir pathway) is dynamic and mirrors growth phases whereas expression from constitutive promoters is constant throughout cultivation. Thus, the regulatory organization of the GAL regulon functions to avoid the burden of unproductive protein synthesis by only expressing the genes for galactose assimilation when it is present. The conservation of cellular resources effected by this inducible phenotype represents a design principle that has also been learned by metabolic engineers. Early observations of growth inhibition resulting from the overproduction of heterologous protein (Kurland and Dong, 1996) led to the use of separate culturing phases for growth and production and strategies for dynamically switching between them (Burg et al., 2016). This concept of dynamic flux control has subsequently been applied at finer resolution to biosynthetic pathways. Engineered circuits that feature a biosensor enable a cell to continually monitor some aspect of its internal state or external environment and automatically adjust their metabolism in response (Hartline et al., 2021), resulting in improvements in titer, rate, and yield (TRY) (Zhang et al., 2012; Xiao et al., 2016; Xu et al., 2014; Dahl et al., 2013). While the use of sugar-responsive biosensors has been proposed to engineer cells to respond to the carbon sources present in a given lignocellulosic hydrolysate for consolidated bioprocessing, the suggested implementations decouple sensing from the broader regulatory and metabolic context in which the sensors evolved (Alvarez-Gonzalez and Dixon, 2019). To our knowledge, our semi-synthetic regulon approach is the first time that this dynamic activation concept has been applied to engineering catabolism in a way that interfaces with native metabolism for the purpose of efficient biomass formation. This coordination is significant as the regulation of CCM has evolved to ensure that resource uptake and utilization are balanced (Nielsen and Keasling, 2016) with the transcription level of a large fraction of the yeast genome (˜15-27%) (Castrillo et al., 2007; Brauer et al., 2008) known to be correlated with growth rate. We, therefore, posit that regulon control of catabolic gene expression, where the amount of carbon fed into CCM is innately con—to the utilization of those resources to match cellular needs, is likely beneficial to fast growth regardless of the carbon source being assimilated.

While we were previously able to engineer a sensor protein variant (Gal3pSyn4.1) capable of strong regulon activation upon induction with xylose, that effort involved an extensive directed evolution campaign that would have to be repeated for each new substrate (Endalur Gopinarayanan and Nair, 2018). Here, we report the construction of a sensor protein variant, Gal3pMC (metabolic coordinator), which robustly activates the GAL regulon on structurally diverse, non-native carbon sources to a similar degree as Gal3pWT upon induction with galactose. Using metadynamics simulations, we show that the most energetically favor-able conformation for Gal3pMC without galactose (MCGal−) is similar to the closed conformational state of Gal3pWT in complex with galactose (WTGal+). In contrast, the free energy surface of Gal3pWT in the absence of galactose (WTGal−) is characterized by a deep energy well reflective of the active site collapsing/misfolding, a conformation that is unproductive for signal transduction. Molecular dynamics (MD) simulations of these three systems reveal large-scale domain movements in the WTGal− system throughout the course of the simulation whereas the WTGal+ and MCGal− systems exhibit much reduced and more localized movement indicating that the mutations carried by Gal3pMC stabilize the protein in a closed state that enables interaction with Gal80p without requiring an inducer. The inducer-independent mechanism of Gal3pMC would normally ablate the dynamic expression profile exhibited by the native, inducer-dependent regulon as it would constantly be turned on. However, we also observed repression of the Gal3pMC-controlled GAL regulon on repressing substrates (glucose, sucrose), which allows for dynamic activation. Gal3pMC thus enables both the dynamic catabolic gene expression and global metabolic remodeling of the semi-synthetic regulon approach to be applied in a substrate-agnostic manner.

Although the inducer-independent activation effected by Gal3pMC represents a departure from the inducer-dependent regulatory logic of the native GAL regulon that served as our inspiration, we, surprisingly, did not observe any major growth defects on either native or non-native substrates compared with a substrate-specific activation approach using Gal3pSyn4.1. In contrast, a ΔGAL80 strain, which represents an alternative approach of achieving substrate-agnostic regulon activation, showed reduced fitness in both conditions, emphasizing the importance of maintaining the native regulatory organization when utilizing a regulon approach. During growth on the native carbon sources glucose, sucrose, and raffinose, the lower fitness likely results from the burden of constitutive GAL regulon activation.

As we previously observed with xylose (Endalur Gopinarayanan and Nair, 2018), using Gal3pMC to implement a regulon approach for growth on arabinose and cellobiose resulted in faster growth rates and higher final cell densities compared to the traditional constitutive over-expression approach despite expressing the same set of catabolic genes. Significantly, the growth rates achieved using this rational approach are comparable or superior to the highest aerobic growth rates for these substrates identified in the literature (Table 1) without the need for time-consuming and/or expensive experiments like ALE (adaptive laboratory evolution), functional genomics, and reverse genetics, or even deletion of genes previously found to mitigate stress of growth on pentoses (e.g., PHO13, ALD6, ASK10, ADH6, COX4, CYC8, etc. (Li et al., 2021)). The success of a semi-synthetic regulon approach in all the single-sugar conditions motivated us to explore their co-utilization as the simultaneous and complete co-utilization of the constituent sugars in lignocellulosic hydrolysates has been identified as essential to their valorization (Gao et al., 2019). One of the major challenges of engineering the co-utilization of lignocellulosic sugars is that the presence of glucose exerts carbon catabolite repression (CCR) in many organisms, hindering their ability to metabolize other sugars until glucose is depleted and repression is relieved (Boles and Hollenberg, 1997; Roca et al., 2004). However, Jin & Cate et al. found that CCR could be avoided by implementing a heterologous pathway for cellobiose, a glucose disaccharide that does not trigger CCR (Galazka et al., 1979; Li et al., 2010). We demonstrate that using Gal3pMC to implement a regulon approach enabled rapid and complete co-utilization of three non-native carbon sources—xylose, arabinose, and cellobiose—in a single strain in only ˜48 h. Despite being evolved for the catabolism of galactose, these results reinforce the universality and robustness of the metabolic coordination effected by the GAL regulon for engineering synthetic hetero-trophy as well as the plasticity of yeast metabolism to diverse substrates. Existing metabolic engineering tools and strategies may remain important. For example, complete utilization of pentoses during co-utilization may require balancing the expression levels of pathway genes. Additionally, while Gal3pMC enables a regulon approach to be rapidly adapted to structurally diverse, non-native carbon sources, it may be dependent on the development and importation of an efficient catabolic module. That said, it may be possible to apply this work to attractive renewable feedstocks like C1 compounds (i.e., CO₂ (Liu et al., 2020), formate, methanol (Antoniewicz, 2019), methane (Clomburg et al., 1979)) as efforts to engineer their assimilation in S. cerevisiae remain nascent. Researchers have noted the potential of platform strains that maximize the concentration of a key precursor to speed the construction and optimization of biosynthetic pathways (Nielsen, 1979). We envision that the integration of substrate catabolism with broader metabolism characteristic of a regulon approach could similarly accelerate the process of engineering catabolism by reducing the number of iterative, semi-rational genetic interventions currently needed to engineer efficient growth on a given non-native substrate.

Together, these results establish the metabolic coordination of a regulon approach as a fast, flexible, and effective strategy for engineering synthetic heterotrophy and provide a tool (Gal3pMC) for adapting it to growth on structurally diverse non-native substrates.

### Materials and Methods

### Strains and Plasmids

TABLE 2
List of strains used.

| Strain | Description |
|---|---|
| W303-1a | MATa leu2-3, 112 trp1-1 can1-100 ura3-1 ade2-1 his3-11, 15 |
| VEG16 | W303-1a ΔGAL3; ΔGRE3; ΔGAL1; ΔGAL7; ΔGAL10 |
| VDT13 | VEG16 lys2::GAL3p-GAL3^{Syn4.1}-TEFt/ADH1t-GAL2-2.1-GAL1/10p-TAL1-HXT7t; |
| | leu2::GAL1p-GAL3^{Syn4.1}-TEFt |
| VDT27 | VEG16 lys2::ADH1t-GAL2^{2.1}-TEF1/TPI1p-TAL1-HXT7t |
| SFS3 | VEG16 lys2::GAL3p-GAL3^{MC}-TEFt; leu2::GAL1p-GAL3^{MC}-TEFt |
| SFS6 | VEG16 lys2::GAL3p-GAL3^{MC}-TEF1t; leu2::GAL1p-GAL3^{MC}-TEFt; his3::HXT7t- |
| | TAL1-GAL1/10p-GAL2^{2.1}-ADH1t |
| SFS11 | VEG16 ΔGAL80; lys2::HXT7t-TAL1-GAL1/10p-GAL2^{2.1}-ADH1t |
| XYL-REG (MC) | SFS6 + pVDT62 |
| ARA-REG (MC) | SFS6 + pVDT38 |
| ARA-CONS | VDT27 + pSFS3 |
| CEL-REG (MC) | SFS3 + pSFS22 |
| CEL-CONS | VEG16 + pSFS10 |

TABLE 3
List of plasmids used.

| Appearance in | | |
|---|---|---|
| Manuscript | Plasmid | Description |
| Expression | pVEG18- | pRS426, 2μ ori, URA3, GAL1p-EGFP-ADH1t |
| dynamics - GAL1p | GAL1p | |
| Expression | pVEG20- | pRS426, 2μ ori, URA3, GAL7p-EGFP-ADH1t |
| dynamics - GAL7p | GAL7p | |
| Expression | pVEG21- | pRS426, 2μ ori, URA3, GAL10p-EGFP-ADH1t |
| dynamics - GAL10p | GAL10p | |
| Expression | pVEG19- | pRS426, 2μ ori, URA3, GAL3p-EGFP-ADH1t |
| dynamics - GAL3p | GAL3p | |
| Expression | pVEG22- | pRS426, 2μ ori, URA3, GAL80p-EGFP-ADH1t |
| dynamics - GAL80p | GAL80p | |
| Expression | pVEG24- | pRS426, 2μ ori, URA3, TEF1p-EGFP-ADH1t |
| dynamics - TEF1p | TEF1p | |
| Expression | pVEG25-TPI1p | pRS426, 2μ ori, URA3, TPI1p-EGFP-ADH1t |
| dynamics - TPI1p | | |
| Expression | pVEG27 | pRS426, 2μ ori, URA3, TDH3p-EGFP-ADH1t |
| dynamics - TDH3p | | |
| Expression | pVEG28 | pRS426, 2μ ori, URA3, GPM1p-EGFP-ADH1t |
| dynamics - GPM1p | | |
| Activation data - | pVEG7 | pRS426, 2μ ori, URA3, ADH1t-EGFP-GAL1p/GAL10p- |
| ΔGAL3 control | | KANMX-HXT7t |
| Activation data - | pVEG8-WT | pVEG7, 2μ ori, URA3, GAL3p-GAL3^{WT}-TEF1t |
| Gal3p^{WT} | | |
| Activation data - | pVEG8-MC | pVEG7, 2μ ori, URA3, GAL3p-GAL3^{MC}-TEF1t |
| Gal3p^{MC} | | |
| Creation of MC | pVDT43 | pIS385, URA3, GAL3p-Gal3p-5.1-Teft |
| integrant strains | | |
| Creation of MC | pVDT44 | pIS376, URA3, GAL1p-Gal3p-5.1-Teft |
| integrant strains | | |
| Creation of pentose | pSFS31 | pIS374, URA3, HXT7t-TAL1-GAL1/10p-GAL2-2.1-ADH1t |
| integrant strains | | |
| pRS426-XYL-REG | pVEG11 | pRS426, 2μ ori, URA3, ADH1t-Piromyces_XYLA*3- |
| | | GAL1p/GAL10p-XKS1-HXT7t |
| pRS426-ARA- | pSFS3 | pRS426, 2μ ori, URA3, ADH1t-araB-TPI1/TEF1p-araA-Hxt7t- |
| CONS | | GPM1p-araD-TEF1t |
| pRS426-ARA-REG | pVDT23 | pRS426, 2μ ori, URA3, ADH1t-araB-GAL1/GAL10p-araA- |
| | | HXT7t-GAL7p-araD-TEF1t |
| pRS426-CEL- | pSFS10 | pRS426, 2μ ori, URA3, ADH1t-gh1-1-TPI1p/TEF1p-cdt-1- |
| CONS | | HXT7t |
| pRS426-CEL-REG | pSFS11 | pRS426, 2μ ori, URA3, ADH1t-gh1-1-GAL1p/GAL10p-cdt-1- |
| | | HXT7t |
| pRS423-XYL-REG | pSFS23 | pRS423, 2μ ori, HIS3, ADH1t-XKS1-GAL1/GAL10p-XYLA- |
| | | HXT7t |
| pRS424-CEL-REG | pSFS22 | pRS424, 2μ ori, TRP1, ADH1t-gh1-1-GAL1/10p-cdt-1-HXT7t |
| pRS425-ARA-REG | pSFS18 | pRS425, 2μ ori, LEU2, TEF1t-araD-GAL7p-HXT7t-araA- |
| | | GAL1/GAL10p-araB-ADH1t |
| BAD | pVDT14 | pRS416, CEN ori, URA3, ADH1t- araB- GAL1p/GAL10p - |
| | | araA-HXT7t-GAL7p-araD-TEFt |

### Media and Transformation

Yeast strains were grown at 30° C. in either defined Synthetic Complete (SC) (Yeast nitrogen base (1.67 g/L), ammonium sulfate (5 g/L), complete supplement mixture without His, Leu, Ura and Trp (0.6 g/L) (Sunrise Science Products, Inc.) or complex Yeast Peptone (YP) medium (20 g/L Yeast Extract, 40 g/L Casein Peptone, 200 mg adenine) medium. SC media was augmented with appropriate nutrients to select for maintenance of plasmids carrying a given auxotrophy. Luria Bertani (LB) broth and LB agar plates with 100 g/mL of ampicillin when required were used for all E. coli propagation and transformation experiments. Commercial E. coli strain NEB-5a (New England Biolabs) was used for MES transformation when constructing plasmids as well as for plasmid propagation. Upon isolation, plasmids were sequenced before being transformed into yeast strains using the protocol of Gietz (2014) and recovered upon agar plates with the appropriate auxotrophy.

### Strain Construction

The yeast strain W303-1a was used for constructing all the strains used in the study. The generation of knockout strain VEG16 from W303-1a was described previously (Endalur Gopinarayanan and Nair, 2018). All subsequent strains were derived from VEG16 via chromosomal integrations using a previously described set of disintegrator plasmids and associated protocols (Sadowski et al., 2007).

### Plasmid Construction

S. cerevisiae promoter, gene, and terminator sequences were amplified from the genome using Phusion Polymerase (ThermoFisher Scientific). XYLA*3 DNA sequence was provided by Prof. Hal S. Alper (University of Texas at Austin). GAL22.1 DNA sequence was provided by Prof. Bernard Hauer (University of Stuttgart, Stuttgart, Germany). Cellobiose utilization genes cdt-1 and ghl-I were provided by Prof. Huimin Zhao (University of Illinois at Urbana-Champaign). Synthesis of DNA primers and Sanger sequencing of plasmid DNA was outsourced to Genewiz. Substrate utilization plasmids were created by combining promoter, catabolic gene, and terminator sequences into previously described yeast shuttle vectors containing either CEN6 (Frazer and O'Keefe, 2007) or 2p (Christianson et al., 1992) origins. All plasmids were constructed using either NEBuilder HiFi DNA Assembly or re-striction enzyme digest and ligation using reagents purchased from New England Biolabs.

### Quantifying Promoter Dynamics Via Fluorescence

Overnight inoculums were grown in 200 μL in 96-well plates containing the required dropout SC medium with glucose (2%) for 24 h. Cultures were then subcultured (5 L) into 200 L SC medium containing the appropriate carbon source (2%) and returned to the incubator. Fluorescence (excitation at 488 nm and emission at 525 nm) and OD₆₀₀ were measured at numerous time points in a Spectramax M3 spectrophotometer to obtain Fluorescence/0D600.

### Molecular Dynamics (MD) Simulations

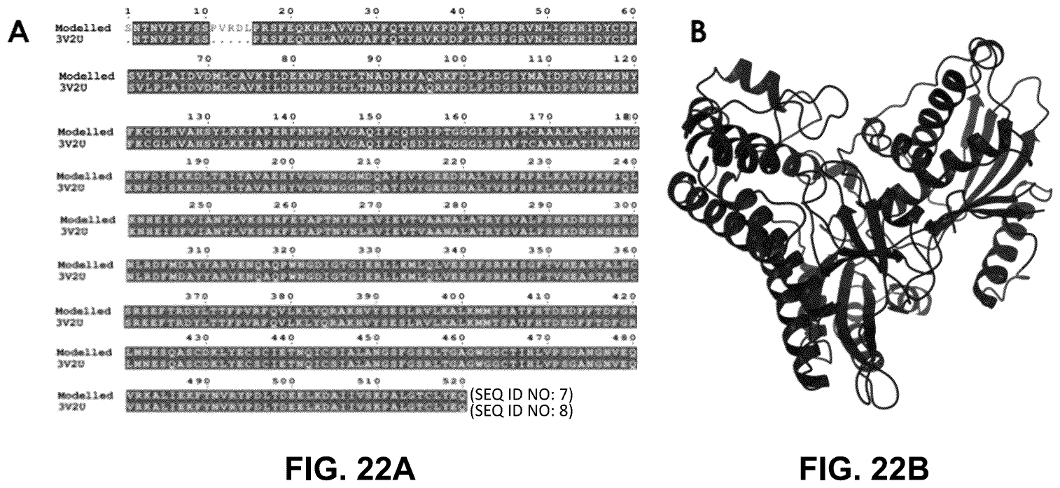

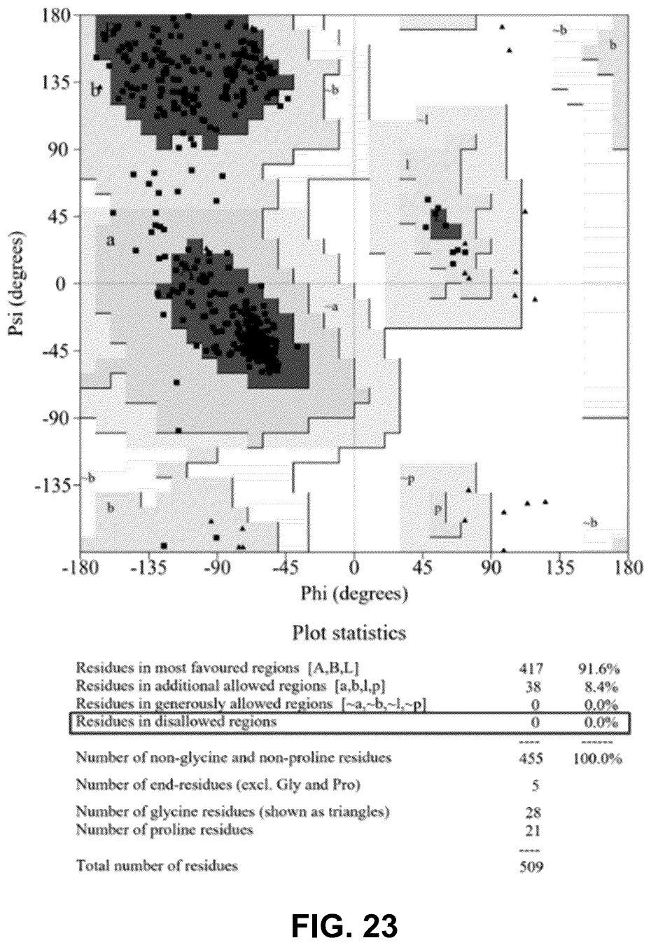

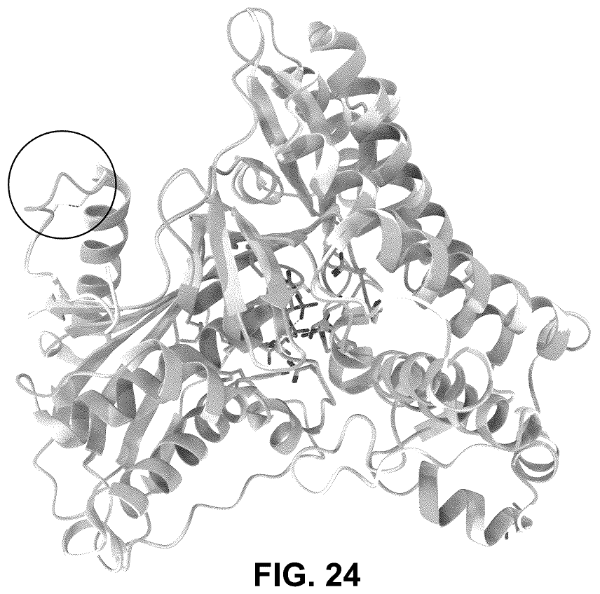

Extended molecular dynamics and metadynamics simulation were conducted on three systems: Gal3pWT complexed with ATP and Mg2+(WTGal−), Gal3pWT complexed with galactose, ATP, and Mg2+(WTGal+), and Gal3pMC complexed with ATP & Mg2+(MCGal−). To establish a common simulation starting point for the three systems, each of the protein structures was modeled into the closed state via homology modeling using Swiss Model (Waterhouse et al., 2018) based on the experimentally determined closed state structure (PDB ID: 3V2U) as a template (FIG. 22). Side chain conformations of the template were retained when generating models. Ramachandran plot was used to confirm that none of the side chains were in disallowed regions (FIG. 23). A comparison of the crystal structure (PDB ID: 3V2U) with the homology model revealed low Root Mean Square Deviation (RMSD) of 0.09 (FIG. 24). Molecular dynamic simulation of the three different experimental setups was performed for 1000 ns using GROMACS-2019.4 (Abraham et al., 2015a). The molecules were parameterized using antechamber package from AMBER (Lindorf- f-Larsen et al., 2010). Quantum polarized charges were derived using the GAMESS package (Guest et al., 1080) with 6-31G basis set and B3LYP level of theory AMBER99SB (Lindorff-Larsen et al., 2010) force field was used to perform a simulation with the TIP3p water model. To solvate the complex, the TIP3P water model with a triclinic box volume of 1000 nm3 was used to mimic or replace the natural environment around the protein. The system was neutralized by adding Na+ ions to the system, 16,574 water molecules and ions overall. Energy minimization of the system was performed using the steepest descent algorithm with convergence energy cut-off of 1000 kJ mol 1 with 1000 steps. Equilibration of the system was performed using NVT and NPT, where NVT is for constant volume and temperature and NPT is for constant pressure. The NVT and NPT were carried out for 500 PS with 300 K temperature using a Berendsen thermostat and the system was prepared for Molecular dynamic simulation for 1 s at 300 K. The system was made to run for 1000 ns, for every 100 ns the trajectory was saved for analysis. The first 100 ns were excluded for every analysis. The distance between C atoms of L370 and D104 has been calculated across the simulation 1000 ns long trajectory for all three proteins to know the variation in the movement of two domain regions across the time. RMSD (Root mean square Deviation) and RMSF (Root mean square fluctuation) of the backbone atoms were calculated over time. Using WORDOM tools (Seeber et al., 2011), cross-correlation studies of residue-residue dis-placements along the trajectory were calculated for each system to quantify dynamic changes of across the moving domains. Similarly, principal component analysis (PCA) was calculated for each 100-1000 ns trajectory and porcupine plots were generated to analyze the intensity and movement of Ca atom. Visualization of structures was done using UCSF Chimera (Pettersen et al., 2004) and Visual Molecular Dynamics (VMD) (Humphrey et al., 1996).

### Metadynamics Simulations

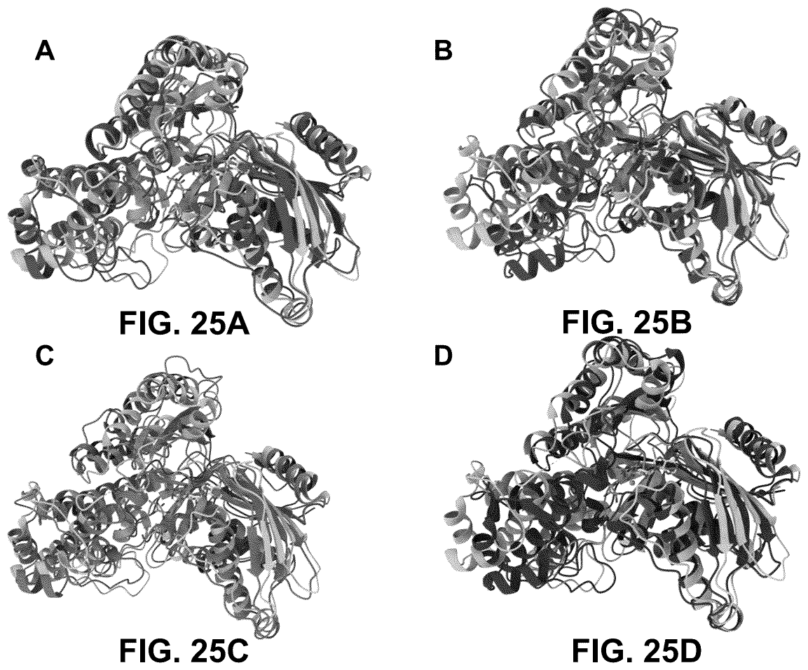

Metadynamics-based simulations use a potential bias to move the system out of a local minimum, which is then used to explore the resulting free energy landscape (FES). Metadynamics-based simulations were conducted using the PLUMED software package (Abrams and Tuckerman, 2008) and GROMACS toolkit for all three experimental setups (WTGal, WTGal+, and MCGal) after thorough equilibration obtained from NVT, NPT ensembles. C1, C2, and C3 are Centers of Mass (COMs) determined for the three regions of Gal3p that fall within the residues regions as shown in FIG. 16A. One of the Collective Variables (CV) for metadynamics simulation was defined as the distance between C1 and C2. The other CV was defined as the angle between the inter-section point of the vectors that pass through C1, C3 and C2, C3, respectively. Different simulations in this study were performed for 50 ns with a bias factor set to 8.0, the temperature set to 300 K, and the results were calculated accordingly. The free energy landscape was explored as the distance between the two domains and the change in the angle formed by the three COMs were varied. A superposition of modeled closed form and apo form was used to visualize the structural changes (FIG. 25).

### Gal3p-Ga80p Docking Studies

Crystal structure of Gal80p complexed with the closed Gal3p structure (PDB ID 3V2U) was used as the reference for protein-protein docking studies. Protein-protein docking studies were conducted using PatchDock (Duhovny et al., 2002; Schneidman-Duhovny et al., 2005) and then refined using Fiberdock (Mashiach et al., 2010a, 2010b). The minimum energy conformations of WTGal+, WTGal−, and MCGal− attained from the metadynamics simulations were docked with Gal80p and the complexes obtained from protein-protein docking studies were simulated using GROMACS-2019.4 (Abraham et al., 2015b) for 50 ns 2 to better equilibrate the complexes that were obtained. The coordinates of the complexes were sampled every 0.02 ns and the binding affinity in kcal/mol between the Gal3p and Gal80p structures for all three complexes was calculated using the molecular mechanics/Poisson-Boltzmann surface area (MM/PBSA) method (Kumari et al., 2014; Baker et al., 2001). PatchDock protocol was per-formed by preparing two PDB files (receptor and ligand), where Gal3p structures from the minimum energy conformations of the metadynamics simulations were used as ligand and Gal80p structure was used as a receptor. Additional parameters were set by giving the binding region as receptor binding site (L44, Q45, S47, S48, P159, R163, Y323, L342, Y346, D347, K350, E351, 1352, M353, E354, V355, Y356, H357, L358, R359, N360, Y361, and R370) and ligand binding site (K81, N82, L98, F99, D100, P102, L103, A109, I110, D111, P112, S113, Y200, E260, T264, V351, N359, S361, R362, E363, E364, T366, R367, L370, T371, T372, P374, V375, R376, F377, Q378, and V379). High accuracy sampling of PatchDock was performed with 1000 conformers. The conformers were clustered according to RMSD with crystal structure as reference and conformations within selected clusters were ranked ac-cording to the ACE (atomic contact energy). The top rigid docking conformer from PatchDock protocol was taken into FiberDock refinement. Hydrogens were added to the receptor (Gal80p). FibreDock refinement of the protein complex was performed with backbone side-chain refinement through normal mode analysis by generating 200 conformers with default parameters. The results were sorted according to ACE score and the best conformer was chosen through visual analysis.

### Growth Studies

Overnight inoculums were grown in the required dropout SC medium with 2% sucrose (unless explicitly stated to be glucose/raffinose) for 24 h. The culture was washed twice in the growth medium (either SC or YP containing 2% of the target sugar) and resuspended at an initial OD₆₀₀ of 0.1 in 250 mL shake flasks containing 20 mL of media. Cell growth OD₆₀₀ measurement was checked at frequent time intervals (3-6 h) on SpectraMax M3 spectrophotometer (Molecular Devices). Growth rates (μ) for each biological replicate were quantified in Microsoft Excel using an exponential curve equation fit to at least three time points during logarithmic growth phase. These values were used to calculate the average growth rate and the standard error for a given condition.

### Residual Substrate Quantification

Residual substrate during co-utilization studies was measured using an Agilent HPLC system equipped with a Hi-Plex H-column and detected using 1260 Agilent ELSD detector. Mobile phase was 0.1% trifluoro-acetic acid (TFA) with a flow rate of 0.6 mL/min. The ELSD detector's nebulizer and evaporation temperature were set at 30° C. and nitrogen flow rate at 1.6 SLM (standard liter per minute). Using these settings, we observed characteristic retention times for cellobiose (9.3 min), arabinose (12.6 min), and xylose (11.8 min).

### REFERENCES

Abraham, M. J., et al., 2015a. GROMACS: high performance molecular simulations through multi-level parallelism from laptops to supercomputers. SoftwareX 1-2, 19-25.
Abraham, M. J., et al., 2015b. GROMACS: high performance molecular simulations through multi-level parallelism from laptops to supercomputers. SoftwareX 1-2, 19-25.
Abrams, J. B., Tuckerman, M. E., 2008. Efficient and direct generation of multidimensional free energy surfaces via adiabatic dynamics without coordinate transformations. J. Phys. Chem. B 112, 15742-15757.
Ajikumar, P. K., et al., 1979. Isoprenoid pathway optimization for Taxol precursor overproduction in Escherichia coli. Science 330, 70-74, 2010.
Alvarez-Gonzalez, G., Dixon, 2019. N. Genetically encoded biosensors for lignocellulose valorization. Biotechnol. Biofuels 12 (1 12), 1-14, 2019.
Antoniewicz, M. R., 2019. Synthetic methylotrophy: strategies to assimilate methanol for growth and chemicals production. Curr. Opin. Biotechnol. 59, 165-174.
Baker, N. A., Sept, D., Joseph, S., Holst, M. J., McCammon, J. A., 2001. Electrostatics of nanosystems: application to microtubules and the ribosome. Proc. Natl. Acad. Sci. USA 98, 10037-10041.
Bhat, P. J., Hopper, J. E., 1992. Overproduction of the GAL1 or GAL3 protein causes galactose-independent activation of the GAL4 protein: evidence for a new model of induction for the yeast GAL/MEL regulon. Mol. Cell Biol. 12, 2701-2707.
Blank, T. E., Woods, M. P., Lebo, C. M., Xin, P., Hopper, J. E., 1997. Novel Gal3 proteins showing altered Gal80p binding cause constitutive transcription of Gal4p-activated genes in Saccharomyces cerevisiae. Mol. Cell Biol. 17, 2566-2575.
Boles, E., Hollenberg, C. P., 1997. The molecular genetics of hexose transport in yeasts. FEMS Microbiol. Rev. 21, 85-111.
Brauer, M. J., et al., 2008. Coordination of growth rate, cell cycle, stress response, and metabolic activity in yeast. Mol. Biol. Cell 19, 352-367.
Burg, J. M., et al., 2016. Large-scale bioprocess competitiveness: the potential of dynamic metabolic control in two-stage fermentations. Curr Opin Chem Eng 14, 121-136.
Castrillo, J. I., et al., 2007. Growth control of the eukaryote cell: a systems biology study in yeast. J. Biol. 6.
   Chen, Y., Stabryla, L., Wei, N., 2016. Improved acetic acid resistance in Saccharomyces cerevisiae by overexpression of the WHI2 gene identified through inverse metabolic engineering. Appl. Environ. Microbiol. 82, 2156-2166.
Christianson, T. W., Sikorski, R. S., Dante, M., Shero, J. H., Heiter, P., 1992. Multifunctional yeast high-copy shuttle vectors. Gene 110, 119-122.
Clomburg, J. M., Crumbley, A. M., Gonzalez, R., 1979. Industrial biomanufacturing: the future of chemical production. Science 355, 2017.
Dahl, R. H., et al., 2013. Engineering dynamic pathway regulation using stress-response promoters. Nat. Biotechnol. 31 (11 31), 1039-1046, 2013.
Deng, J., et al., 2021. A synthetic promoter system for well-controlled protein expression with different carbon sources in Saccharomyces cerevisiae. Microb. Cell Factories 20, 1-10.
Duhovny, D., Nussinov, R., Wolfson, H. J., 2002. Efficient unbound docking of rigid molecules. Lect. Notes Comput. Sci. 2452, 185-200.
Eiteman, M. A., Lee, S. A., Altman, E., 2008. A co-fermentation strategy to consume sugar mixtures effectively. J. Biol. Eng. 2, 1-8.
Endalur Gopinarayanan, V., Nair, N. U., 2018. A semi-synthetic regulon enables rapid growth of yeast on xylose. Nat. Commun. 9.
Flores, A. D., et al., 2020. Catabolic division of labor enhances production of D-lactate and succinate from glucose-xylose mixtures in engineered Escherichia coli Co-culture systems. Front. Bioeng. Biotechnol. 8, 1-14.
Frazer, L. N., O'Keefe, R. T., 2007. A new series of yeast shuttle vectors for the recovery and identification of multiple plasmids from Saccharomyces cerevisiae. Yeast 24, 777-789.
Galazka, J. M., et al., 1979. Cellodextrin transport in yeast for improved biofuel production. Science 330, 84-86, 2010.
Gao, M., Ploessl, D., Shao, Z., 2019. Enhancing the Co-utilization of biomass-derived mixed sugars by yeasts. Front. Microbiol. 9, 1-21.
Gietz, R. D., 2014. Yeast transformation by the LiAc/SS carrier DNA/PEG method. Yeast Protocols 1163 33-44.
Gopinarayanan, V. E., Nair, N. U., 2018. A semi-synthetic regulon enables rapid growth of yeast on xylose. Nat. Commun. 9.
Griffin, T. J., et al., 2002. Complementary profiling of gene expression at the transcriptome and proteome levels in Saccharomyces cerevisiae. Mol. Cell. Proteomics 1, 323-333.
Guest, M. F. et al. The GAMESS-UK electronic structure package: algorithms, developments and applications. 103, 719-747 (2007).
Ha, S. J., et al., 2011. Engineered Saccharomyces cerevisiae capable of simultaneous cellobiose and xylose fermentation. Proc. Natl. Acad. Sci. U.S.A 108, 504-509.
HamediRad, M., Lian, J., Li, H., Zhao, H., 2018. RNAi assisted genome evolution unveils yeast mutants with improved xylose utilization. Biotechnol. Bioeng.
Hartline, C. J., Schmitz, A. C., Han, Y., Zhang, F., 2021. Dynamic control in metabolic engineering: theories, tools, and applications. Metab. Eng. 63, 126-140.
Humphrey, W., Dalke, A., Schulten, K., 1996. VMD: visual molecular dynamics. J. Mol. Graph. 14, 33-38.
Kim, S. R., et al., 2013a. Rational and evolutionary engineering approaches uncover a small set of genetic changes efficient for rapid xylose fermentation in Saccharomyces cerevisiae. PLoS One 8, e57048.
Kim, B., Du, J., Eriksen, D. T., Zhao, H., 2013b. Combinatorial design of a highly efficient xylose-utilizing pathway in Saccharomyces cerevisiae for the production of cellulosic biofuels. Appl. Environ. Microbiol. 79, 931-941.
Kobayashi, Y., Sahara, T., Ohgiya, S., Kamagata, Y., Fujimori, K. E., 2018. Systematic optimization of gene expression of pentose phosphate pathway enhances ethanol production from a glucose/xylose mixed medium in a recombinant Saccharomyces cerevisiae. Amb. Express 8, 1-11.
Kumari, R., Kumar, R., Lynn, A., 2014. g_mmpbsa—a GROMACS tool for high-throughput MM-PBSA calculations. J. Chem. Inf. Model. 54, 1951-1962.
Kurland, C. G., Dong, H., 1996. Bacterial growth inhibition by overproduction of protein. Mol. Microbiol. 21, 1-4.
Langholtz, M., Stokes, B., Eaton, L., 2016. billion-ton report: advancing domestic resources for a thriving bioeconomy (Executive Summary). Ind. Biotechnol. 12, 282-289, 2016.
Latimer, L. N., et al., 2014. Employing a combinatorial expression approach to characterize xylose utilization in Saccharomyces cerevisiae. Metab. Eng. 25, 20-29.
Lavy, T., Kumar, P. R., He, H., Joshua-Tor, L., 2012. The Gal3p transducer of the GAL regulon interacts with the Gal80p repressor in its ligand-induced closed conformation. Genes Dev. 26, 294-303.
Lee, S. M., Jellison, T., Alper, H. S., 2012. Directed evolution of xylose isomerase for improved xylose catabolism and fermentation in the yeast Saccharomyces cerevisiae. Appl. Environ. Microbiol. 78, 5708-5716.
Li, S., et al., 2010. Overcoming glucose repression in mixed sugar fermentation by co-expressing a cellobiose transporter and a P-glucosidase in Saccharomyces cerevisiae. Mol. Biosyst. 6, 2129-2132.
Li, X., et al., 2021. Metabolic network remodelling enhances yeast's fitness on xylose using aerobic glycolysis. Nat Catal 4.
Lindorff-Larsen, K., et al., 2010. Improved side-chain torsion potentials for the Amber ff99SB protein force field. Proteins: Struct., Funct., Bioinf. 78, 1950-1958.
Liu, Z., Wang, K., Chen, Y., Tan, T., Nielsen, J., 2020. Third-generation biorefineries as the means to produce fuels and chemicals from CO2. Nat Catal 3, 274-288.
Malakar, P., Venkatesh, K. V., 2014. GAL regulon of Saccharomyces cerevisiae performs optimally to maximize growth on galactose. FEMS Yeast Res. 14, 346-356.
Mashiach, E., Nussinov, R., Wolfson, H. J. FiberDock, 2010a. Flexible induced-fit backbone refinement in molecular docking. Proteins 78, 1503-1519.
Mashiach, E., Nussinov, R., Wolfson, H. J. FiberDock, 2010b. A web server for flexible induced-fit backbone refinement in molecular docking. Nucleic Acids Res. 38, W457-W461.
Mukherjee, V., Lind, U., St Onge, R. P., Blomberg, A., Nygird, Y., 2021. A CRISPR interference screen of essential genes reveals that proteasome regulation dictates acetic acid tolerance in Saccharomyces cerevisiae. mSystems 6.
Ni, H., Laplaza, J. M., Jeffries, T. W., 2007. Transposon mutagenesis to improve the growth of recombinant Saccharomyces cerevisiae on D-xylose. Appl. Environ. Microbiol. 73, 2061-2066.
Nielsen, J., 1979. Yeast cell factories on the horizon: metabolic engineering in yeast gets increasingly more versatile. Science 349, 1050-1051, 2015.
Nielsen, J., Keasling, J. D., 2016. Engineering cellular metabolism. Cell 164, 1185-1197.
Peng, B., Williams, T. C., Henry, M., Nielsen, L. K., Vickers, C. E., 2015. Controlling heterologous gene expression in yeast cell factories on different carbon substrates and across the diauxic shift: a comparison of yeast promoter activities. Microb. Cell Factories 14, 1-11.
Pettersen, E. F., et al., 2004. UCSF Chimera—a visualization system for exploratory research and analysis. J. Comput. Chem. 25, 1605-1612.
Protzko, R. J., et al., 2018. Engineering Saccharomyces cerevisiae for co-utilization of d-galacturonic acid and d-glucose from citrus peel waste. Nat. Commun. 9, 5059.
Quarterman, J., et al., 2016. Rapid and efficient galactose fermentation by engineered Saccharomyces cerevisiae. J. Biotechnol. 229, 13-21.
Reimand, J., Vaquerizas, J. M., Todd, A. E., Vilo, J., Luscombe, N. M., 2010. Comprehensive reanalysis of transcription factor knockout expression data in Saccharomyces cerevisiae reveals many new targets. Nucleic Acids Res. 38, 4768-4777.
Ren, B., et al., 1979. Genome-wide location and function of DNA binding proteins. Science 290, 2306-2309, 2000.
Reznicek, O., et al., 2015. Improved xylose uptake in Saccharomyces cerevisiae due to directed evolution of galactose permease Gal2 for sugar co-consumption. J. Appl. Microbiol. 119, 99-111.
Roca, C., Haack, M. B., Olsson, L., 2004. Engineering of carbon catabolite repression in recombinant xylose fermenting Saccharomyces cerevisiae. AppL. Microbiol. Biotechnol. 63, 578-583.
Rogers, J. N., et al., 2017. An assessment of the potential products and economic and environmental impacts resulting from a billion ton bioeconomy. Biofuels, Bioproducts and Biorefining 11, 110-128.
Sadowski, I., Su, T. C., Parent, J., 2007. Disintegrator vectors for single-copy yeast chromosomal integration. Yeast 24, 447-455.
Sanchez, R. G., et al., 2010. Improved xylose and arabinose utilization by an industrial recombinant. Biotechnol. Biofuels 3.
Schneidman-Duhovny, D., Inbar, Y., Nussinov, R., Wolfson, PatchDock, H. J., SymmDock, 2005. Servers for rigid and symmetric docking. Nucleic Acids Res. 33, W363-W367. Seeber, M., et al., 2011. Wordom: a user-friendly program for the analysis of molecular structures, trajectories, and free energy surfaces. J. Comput. Chem. 32, 1183-1194.
Smanski, M. J., et al., 2014. Functional optimization of gene clusters by combinatorial design and assembly. Nat. Biotechnol. 32, 1241-1249.
Torchia, T. E., Hamilton, R. W., Cano, C. L., Hopper, J. E., 1984. Disruption of regulatory gene GAL80 in Saccharomyces cerevisiae: effects on carbon-controlled regulation of the galactose/melibiose pathway genes. Mol. Cell Biol. 4, 1521-1527.
Trivedi, V. D., Sullivan, S. F., Choudhury, D., Endalur Gopinarayanan, V., Nair, N. U., 2022. Integration of metabolism and regulation reveals rapid adaptability to growth on non-native substrates. bioRxiv.
Unrean, P., G'atgens, J., Klein, B., Noack, S., Champreda, V., 2018. Elucidating cellular mechanisms of Saccharomyces cerevisiae tolerant to combined lignocellulosic- derived inhibitors using high-Throughput phenotyping and multiomics analyses. FEMS Yeast Res. 18, 1-10.
Venturelli, O. S., El-Samad, H., Murray, R. M., 2012. Synergistic dual positive feedback loops established by molecular sequestration generate robust bimodal response. Proc. Natl. Acad. Sci. U.S.A 109.
Verhoeven, M. D., de Valk, S. C., Daran, J. M. G., van Maris, A. J. A., Pronk, J. T., 2018. Fermentation of glucose-xylose-Arabinose mixtures by a synthetic consortium of single-sugar-fermenting Saccharomyces cerevisiae strains. FEMS Yeast Res. 18, 1-12.
Waterhouse, A., et al., 2018. SWISS-MODEL: homology modelling of protein structures and complexes. Nucleic Acids Res. 46. W296-W303.
Wisselink, H. W., et al., 2007. Engineering of Saccharomyces cerevisiae for efficient anaerobic alcoholic fermentation of L-arabinose. Appl. Environ. Microbiol. 73, 4881-4891.
Xiao, Y., Bowen, C. H., Liu, D., Zhang, F., 2016. Exploiting nongenetic cell-to-cell variation for enhanced biosynthesis. Nat. Chem. Biol. 12 (5 12), 339-344, 2016.
Xu, P., Li, L., Zhang, F., Stephanopoulos, G., Koffas, M., 2014. Improving fatty acids production by engineering dynamic pathway regulation and metabolic control. Proc. Natl. Acad. Sci. U.S.A 111, 11299-11304.
Zhang, F., Carothers, J. M., Keasling, J. D., 2012. Design of a dynamic sensor-regulator system for production of chemicals and fuels derived from fatty acids. Nat. Biotechnol. 30 (4 30), 354-359, 2012.
Zhou, H., Cheng, J. S., Wang, B., Fink, G. R., Stephanopoulos, G., 2012. Xylose isomerase overexpression along with engineering of the pentose phosphate pathway and evolutionary engineering enable rapid xylose utilization and ethanol production by Saccharomyces cerevisiae. Metab. Eng. 14, 611-622.

### Example 2: Integration of Metabolism and Regulation Reveals Rapid Adaptability to Growth on Non-Native Substrates

Engineering synthetic heterotrophy is a key to the efficient bio-based valorization of renewable and waste substrates. Among these, engineering hemicellulosic pentose utilization has been well-explored in Saccharomyces cerevisiae (yeast) over several decades-yet the answer to what makes their utilization inherently recalcitrant remains elusive. Through implementation of a semi-synthetic regulon, we find that harmonizing cellular and engineering objectives are a key to obtaining highest growth rates and yields with minimal metabolic engineering effort. Concurrently, results indicate that “extrinsic” factors-specifically, upstream genes that direct flux of pentoses into central carbon metabolism—are rate-limiting. We also reveal that yeast metabolism is innately highly adaptable to rapid growth on non-native substrates and that systems metabolic engineering (i.e., functional genomics, network modeling, etc.) is largely unnecessary. Overall, this work provides an alternate, novel, holistic (and yet minimalistic) approach based on integrating non-native metabolic genes with a native regulon system.

### Introduction

Engineering metabolism for growth on non-native substrates (i.e., synthetic heterotrophy) has been an outstanding challenge for several decades in various microbial species.¹⁻⁴ The traditional approach is to constitutively overexpress catabolic genes that input substrate to central carbon metabolism with the expectation that the native cellular systems can direct subsequent steps required for growth. In case initial designs do not lead to rapid growth, flux balancing,⁵ adaptive laboratory evolution (ALE),^{6,7} functional genomics,^{8,9} and directed evolution^{6,10} approaches are used. More recently, systems-level analysis of regulatory structures in engineered strains has been used to identify dysregulated pathways and corrective interventions have provided avenues to improve engineering outcomes.¹¹⁻¹³ Unifying to these approaches is that they are reactive interventions that try to rectify inefficiencies introduced due to strain engineering but are not proactive in circumventing undesirable outcomes by harmonizing recombinant activity with native cellular objectives. Typical of this approach is engineering the yeast Saccharomyces cerevisiae for complete and rapid utilization of lignocellulosic pentoses.¹⁴⁻¹⁷ In this work, we engineer strains of yeast for rapid growth and pentose (xylose, arabinose) utilization and assess intrinsic and extrinsic constraints and pitfalls that control desirable phenotypic outcomes. Specifically, we answer the longstanding unresolved question: what are the inherent intrinsic limitations within this yeast that prevent it from metabolizing non-native pentose substrates at rates equivalent to native substrates?Surprisingly, we find that this yeast is intrinsically highly adaptable to rapid growth on non-native substrates and that it can achieve maximum growth rates with minimal engineering—which is in direct contrast with the currently accepted paradigm. To achieve this, we first modified our semi-synthetic regulon¹⁸—a system based on synthetically activating the galactose (GAL) response system using a non-native substrate—for efficient utilization of arabinose through the isomerase pathway.¹⁹ We demonstrate that while initial outcomes—like with xylose, previously—are superior to constitutively overexpressing the same genes, growth rates were modest (m=0.14-0.17 h⁻¹). To identify factors that constrain growth on either pentose, we performed pathway balancing, directed evolution, and systems biology-driven functional genomics. To our surprise, we found that when cells can coordinate global growth responses with substrate use through the semi-synthetic GAL regulon (REG), growth is largely extrinsically controlled—i.e., limited by the upstream/heterologous pathway that controls non-native substrate uptake and flux to central carbon metabolism. We also found through parallel investigations that genetic interventions identified through traditional/systems metabolic engineering that prune cellular metabolic and/or regulatory networks to improve strain performance often led to pleiotropic defects with mid-to-severe fitness costs. Thus, our findings suggest that an approach that synergizes activation of the GAL regulon with an optimized upstream heterologous metabolic module reveals the hidden metabolic adaptability of yeast.

### Results

### A Semi-Synthetic Regulon Design Outperforms a Constitutive Overexpression Strategy

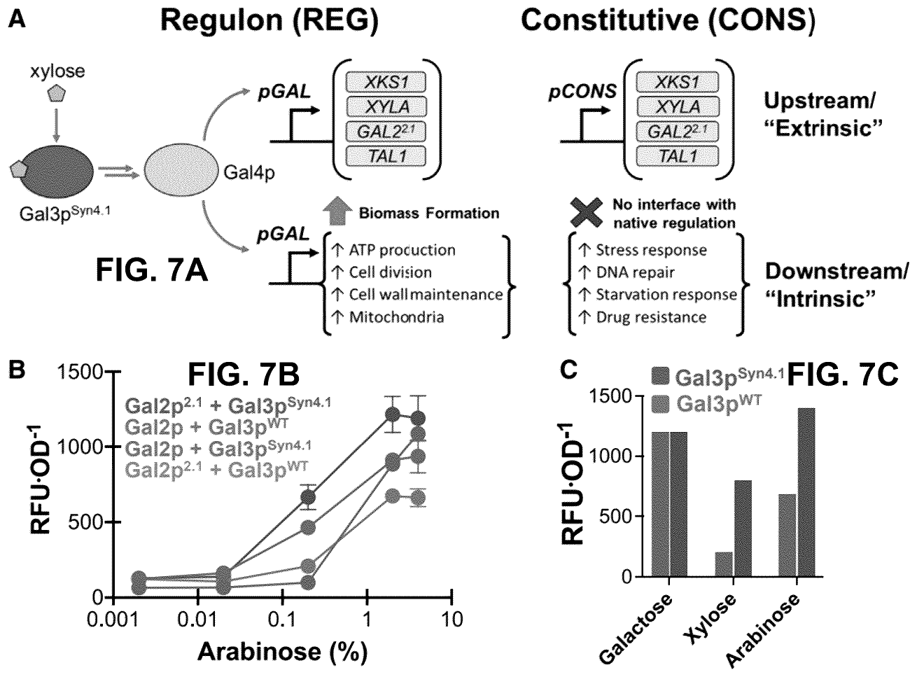

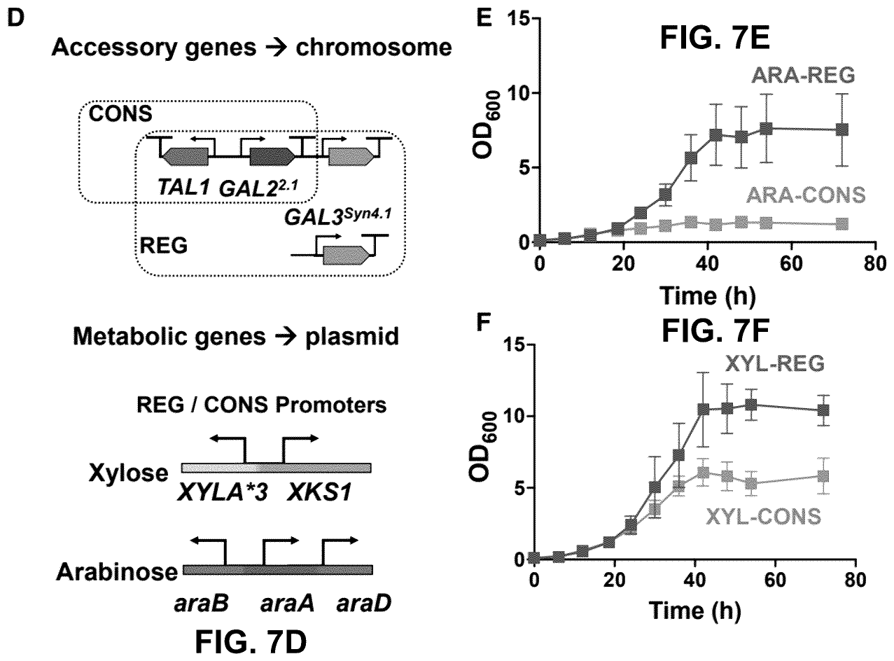

Previously, we demonstrated that coupling activation of galactose (GAL)-responsive regulon to catabolism and growth on non-native substrate, xylose, enabled faster growth and more complete utilization when compared to the constitutive overexpression of the same genes.¹⁸ This is because GAL regulon activation upregulates growth-responsive genes and suppresses starvation responses, in contrast to the traditional constitutive overexpression approaches where the opposite holds true (FIG. 7A).¹⁸ We wanted to assess whether the benefit was unique to xylose or if growth on other substrates, like arabinose, could also benefit from activation of the GAL regulon. Since Gal3pSyn4.1 was engineered to activate the GAL regulon in a xylose-dependent manner, we wondered if arabinose could do so as well since the two sugars are structurally similar. We quantified activation of GAL1p-EGFP by wild-type Gal3p or engineered Gal3pSyn4.1 in the presence of native Gal2p and/or engineered Gal2p2.1 permease.18 We observed that a Gal3pSyn4.1 and Gal2p2.1 co-expressing strain showed activation on arabinose with low background and high-dynamic range (FIG. 7B). Interestingly, arabinose showed higher activation of GAL1p-EGFP than xylose, even though both Gal3pSyn4.1 and Gal2p2.1 were engineered for activity on xylose (FIG. 7C). Given robust activation, we constructed a semi-integrant strain by integrating accessory genes including, sensor (GAL3Syn4.1), transporter (GAL22.1), and transaldolase (TAL 1) expressed under GAL promoters- to generate an “REG” (regulon) strain background (FIG. 7D). Similarly, as control, and to compare with the traditional engineering approach, we integrated GAL22.1 and TAL1 under strong constitutive promoters to generate the “CONS” parental strain.

We then assessed the growth of both strains on arabinose or xylose after transformation with plasmid-encoded araBAD or XYLA*3-XKSI catabolic genes, respectively. In ARA-REG and XYL-REG all genes were expressed under GAL-responsive promoters whereas in ARA-CONS and XYL-CONS all genes were under constitutive promoters (FIG. 7D). On both carbon sources, strains that used the GAL regulon to coordinate substrate assimilation with global metabolism demonstrated higher growth rates (m: ARA-REG=0.14±0.04 h⁻¹, XYL-REG=0.17±0.01 h⁻¹) compared to those that constitutively overexpressed the same genes (m: ARA-CONS=0.06±0.01 h⁻¹, XYL-CONS=0.11±0.01 h⁻¹). They also reached higher final cell densities (OD₆₀₀: ARA-REG=6.01±1.62, ARA-CONS=1.31±0.04, XYLREG=10.8±0.62, XYL-CONS=5.3±0.49) (FIGS. 7E and F). In general, overexpression of heterologous proteins, especially membrane proteins, can be toxic to the host. However, this is not the case in our study since the use of very strong GAL-responsive promoters for TAL1 and GAL2^{2.1} promoted growth on pentoses. We used strong constitutive promoters TEF1p and TPI1p in CONS strains rather than weaker promoters to maximize/match expression. However, it seems that the absence of dynamics and weaker total activation may have contributed to poorer growth of the CONS strains.²⁰⁻²²

We then wanted to assess what factors prevented these REG strains from achieving maximum aerobic growth rate (m=0.22-h⁻¹).^{13,23} We explored two potential limitations i) intrinsic, i.e., native metabolic or regulatory systems downstream of substrate uptake, and ii) extrinsic, i.e., upstream metabolic modules (primarily heterologous genes) that direct non-native substrates to central carbon metabolism.

### Perturbation of Intrinsic (Downstream) Factors Result in Modest and Inconsistent Improvements

In traditional/systems metabolic engineering strategies, adaptive laboratory evolution (ALE) or rational data-driven approaches are often used to improve growth rates of initial strain designs, since growth rates and biomass yields are most often suboptimal. We decided to use a (data-driven) systems biology approach to identify “intrinsic” genetic targets to modify to improve the growth rates of our REG strains on xylose and arabinose (i.e., pentoses). By “intrinsic” factors, we mean native yeast genes that are far downstream of substrate uptake and catabolism (e.g., cell wall biosynthesis, DNA repair).

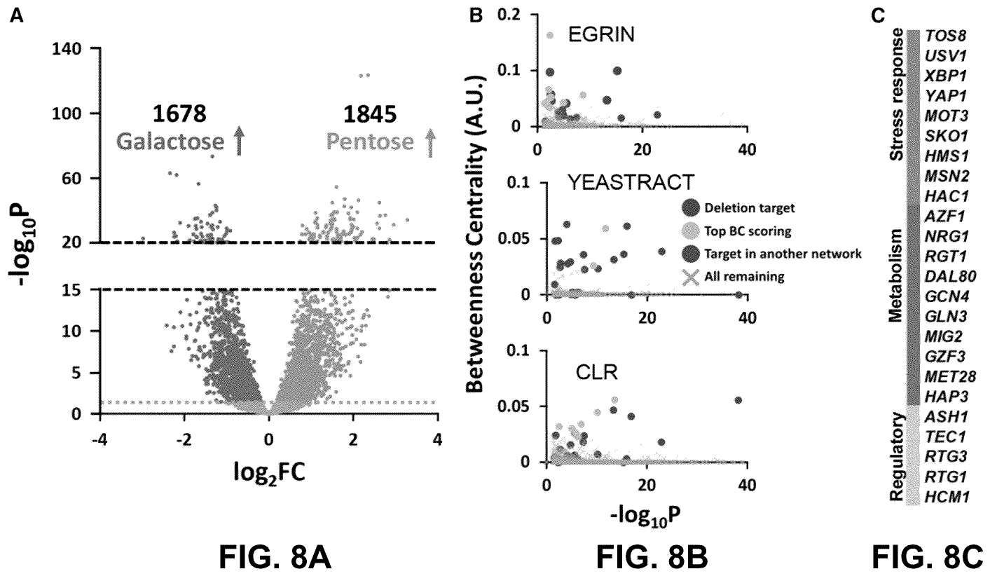

As the GAL regulon has evolved to function harmoniously during growth on a native substrate, we hypothesized that identifying expression differences between galactose and the two non-native substrates and closing the gap between them may aid in better synchronizing GAL regulation with pentose metabolism. To do so, we performed RNA-seq on REG strains grown on arabinose, xylose, and galactose to characterize their respective expression profiles. A differential gene expression (DGE) analysis between individual sugars revealed that a total of 838 genes were differentially regulated between arabinose and galactose and 1,430 genes between xylose and galactose strains (p value <0.05 after Benjamini-Hochberg correction). However, rather than pursue the individual substrate comparisons, we chose to repeat the DGE analysis to compare pentose REG vs. galactose utilization to identify “intrinsic” factors that differentiate growth on the two pentoses vs. the hexose galactose. Upon doing so, we found 1,845 genes that were relatively upregulated on pentoses and 1,678 genes that were relatively upregulated during growth on galactose (FIG. 8A). Given the large number of differentially expressed genes (3,523) between the two conditions (pentoses vs. galactose), we sought to use gene regulatory networks (GRN) to identify highly connected, regulatory elements that could serve as major contributors to the observed divergence in transcriptional profiles and, therefore, growth rates. To do so, we first identified three different yeast GRNs that were previously validated for their ability to improve the accuracy of yeast growth phenotypes when integrated with existing metabolic models.²⁴ These GRNs EGRIN, YEASTRACT, and CLR all sought to capture influences on expression between yeast genes but differed in the data types (gene expression, DNA-binding, protein-protein interactions, etc.) used to infer these connections. The resulting networks were represented as graphs with nodes corresponding to genes and edges between nodes corresponding to an inferred connection between genes. To utilize these networks, we began by associating each node with the expression fold-change and (Benjamini-Hochberg corrected) significance values from the differential expression comparison between the pentose and galactose-grown REG strains. We then eliminated all nodes with corrected p values >0.05 along with any connections to those nodes to isolate significantly perturbed sections of the network. We next utilized the network visualization and analysis software tool Cytoscape²⁵ to assign a betweenness centrality (BC), a measure of the centrality of a given node in a network, value to the remaining genes in each network. Plotting BC vs. significance in gene expression change helped narrow down a subset of regulatory genes as targets for deletion (FIG. 8B). Finally, we manually curated a subset of 24 genes among these that have roles in regulation, metabolism, and stress response for deletion to test their effect of growth and phenotype (FIG. 8C).

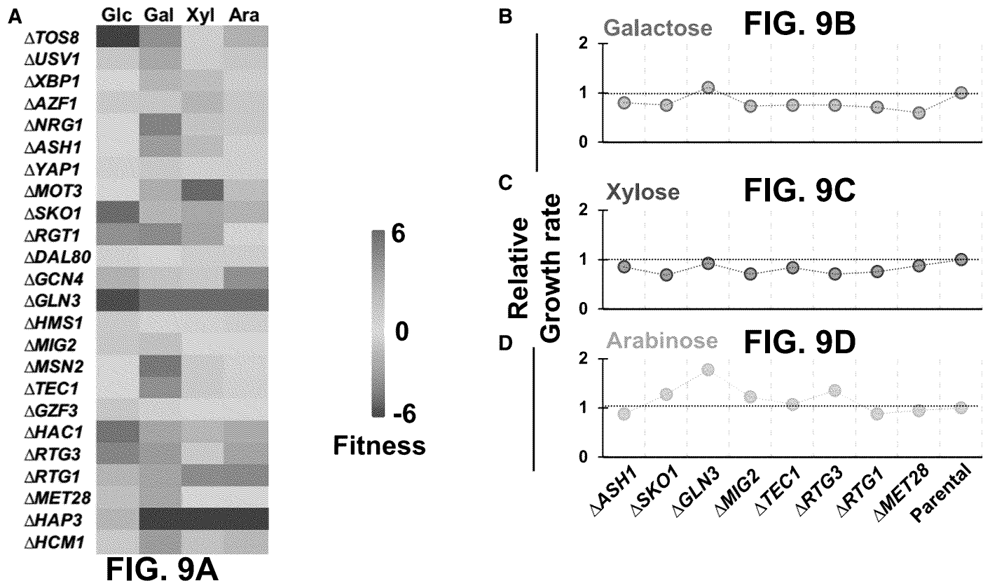

We generated barcoded deletion (KO) libraries of these “intrinsic” factors in wild-type and REG strain backgrounds and performed growth enrichments on glucose, galactose, xylose, and arabinose (FIG. 9A). Comparing population shifts using barcodes, we identified eight genes that displayed positive fitness on both xylose and arabinose but not on galactose-indicating a role in controlling growth primarily on pentoses. We then tested their growth individually on galactose, xylose, and arabinose (FIG. 3B-D) and found that only DGLN3 was either neutral or beneficial on both pentoses-all other deletions were detrimental for growth on xylose. Further, the growth rates of all knockout strains were still lower than that of the parental strain on galactose (m=0.24±0.03 h⁻¹ when GAL1-7-10 are expressed from a plasmid), indicating additional limitations. Overall, the systems metabolic engineering approach failed to identify genetic factors broadly beneficial to growth on pentoses. In contrast, the genome-wide expression changes affected by GAL regulon activation are beneficial for all three substrates.

Pentose Metabolism is Largely Extrinsically (Upstream) Controlled with a Regulon Approach

Our studies have so far focused on identifying “intrinsic” factors that may be controlling/bottlenecking growth on pentoses and given the inconsistent benefits, we wondered whether the limitations were, in fact, “extrinsic”. The former implies that the native metabolic and/or regulatory capacity of this yeast is inherently limited for effective pentose metabolism and improving growth rate requires vast restructuring of associated (intrinsic) metabolic and/or regulatory networks. The latter implies that there are no inherent limitations in this yeast and that it is already poised for rapid growth on pentoses, but the observed low growth rates are due to suboptimal design of the upstream metabolic module that includes the heterologous (extrinsic) genes. To assess whether the “extrinsic limitation” paradigm has any merits, we needed to optimize the design of the upstream metabolic module responsible for substrate uptake and flux into central carbon metabolism (i.e., glycolysis).

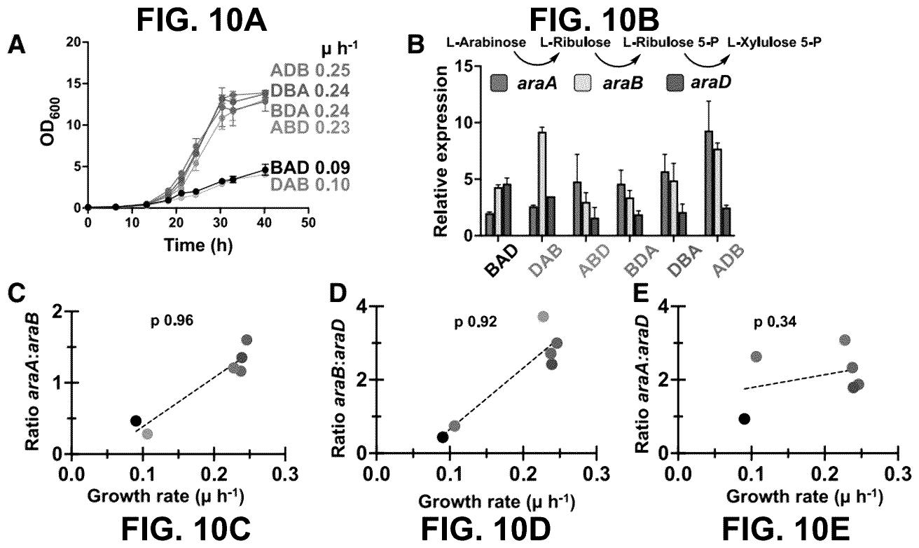

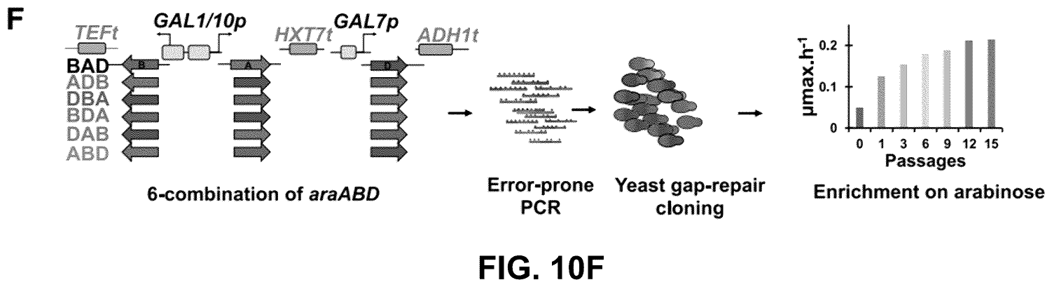

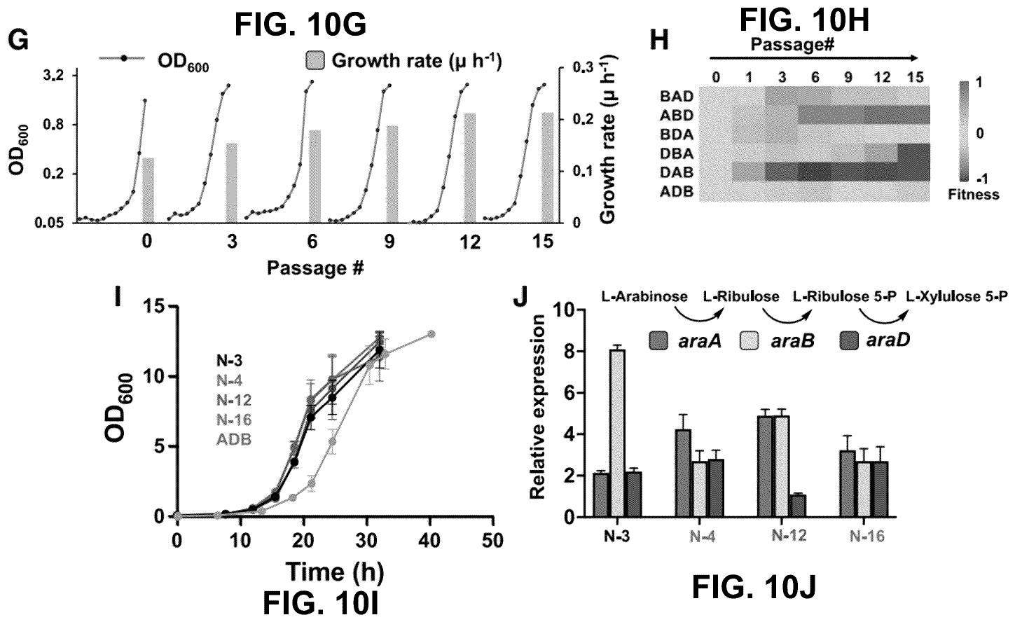

First, we looked at the effect of plasmid copy number. For arabinose, there was no difference in growth rate when araAaraB-araD genes were expressed on high- or low-copy plasmids, whereas for xylose, we only observed growth when the XYLA*3-XKSI gene dose was high. In either case, changing plasmid backbone did not improve growth rate. Next, we hypothesized that balancing expression of these heterologous genes may be required to enhance growth rate. For arabinose, we created all six combinations of gene-promoter pairings and assessed their performance. While such promoter swaps have been previously demonstrated to improve strain performance,²⁶ we were surprised at the marked improvement in growth rate—from 0.14±0.04 h⁻¹ in the original design to 0.27±0.03 h⁻¹ in the best re-design (FIG. 10A). To understand the cause of this behavioral change, we compared the relative expression levels of the araA, araB, and araD using quantitative reverse transcription PCR (qPCR) (FIG. 10B). We observed that in the poor performing combinations, expression of araB (ribulokinase) was the highest, whereas, in high-performing combinations, expression level followed this pattern araA >araB >araD. In addition, we found a strong positive correlation between growth rate and relative expression of araA:araB and araA:araD, respectively (FIG. 10C-E). The success of this approach encouraged us to attempt the same on xylose and we found similar improvements—from 0.17±0.01 h⁻¹ to 0.24±0.01 h⁻¹. These are comparable to the growth rate of yeast on galactose when GAL1-7-10 are expressed from a plasmid (0.24±0.03 h⁻¹).

Finally, we used directed evolution to improve the growth rate on arabinose further (FIG. 10F). We randomly mutagenized the six arabinose pathway combinations, adding barcodes to track the lineages and enriched this library size of 108 variants in minimal (SC+Ara) and complex (2YP+Ara) arabinose media (FIG. 4F). Over the course of 15 subcultures, the growth rate over subcultures increased from 0.12 h⁻¹ to 0.22 h⁻¹ and 0.18 h⁻¹ to 0.26 h⁻¹ in SC+Ara and 2YP+Ara, respectively (FIG. 10G). Using barcodes, we tracked the performance of promoter-gene combinations (FIG. 10H). We observed that initially all the six plasmids start at similar abundance, but the araA-B-D (under GAL1-10-7p, respectively) was the most abundant at the end of enrichment in SC+Ara (FIG. 4H). We picked single colonies from each condition and calculated their growth rates and identified 4 variants that showed the highest growth rates (0.35±0.04 h⁻¹) from the SC+Ara enriched culture. We sequenced the barcodes to identify the lineage and the whole cassette to identify the mutations and found that three of the six initial designs were represented in the four best variants (named N-3, N-6, N-12, and N-16) (Table 3). Re-transformation into parent background strain indicated that the four variants attained the same maximum growth rate as that on galactose (0.29±0.01 h⁻¹), which is the highest reported growth rate on any pentose reported in the literature for this yeast (FIG. 10I). Since the mutations were distributed throughout the cassettes, we quantified the expression levels of araA, araB, and araD in the four strains (FIG. 10J) and found patterns that differed significantly than parental strains (FIG. 10B). We saw a strong positive correlation between growth rate and relative expression of araA:araB and negative correlation between araB:araD, indicating that the directed evolution campaign likely altered both activity and expression in each strain differently. These results highlight a key insight about yeast: its ability to utilize pentoses is largely limited only by “extrinsic” factors (upstream pathway, especially heterologous enzyme activity) and minimally by any “intrinsic” factor (i.e., native regulation or metabolic pathway).

### Engineering Intrinsic (Downstream) Genes Lead to Pleiotropic Fitness Trade-Offs

Next, we assessed whether the global regulatory elements identified through our network analysis would benefit our best designs (araA-B-D under GAL1-10-7p and XYLA*3-XKSI under GAL1-7p) but found that deletion of each of the 8 genes did not result in any improvements over the parental strains (FIG. 5A). We considered exploring combinatorial deletions and ALE to attempt to further enhance growth rates on these two substrates. However, we expected that significant improvements were unlikely as both strains had already attained rates comparable to the empirically determined maximum aerobic growth rates of this yeast (0.22 h⁻¹-0.29 h⁻¹)^{13,23} with high biomass yields and short lag, we expect that improvements would be insignificant.

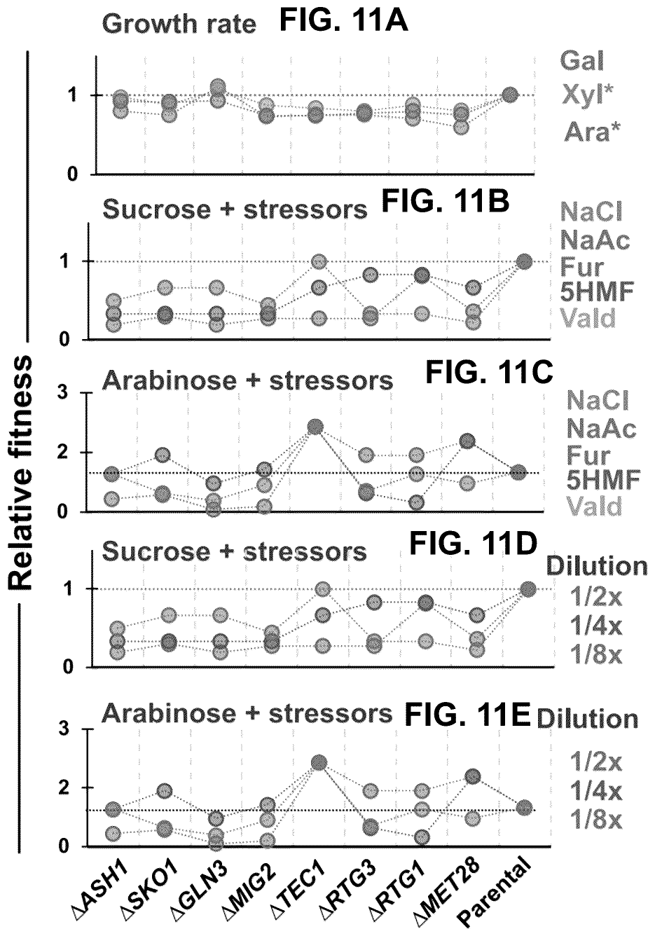

One consideration not yet assessed is the suitability of these strains for bioprocessing and their resilience to growth inhibitors associated with bioprocessing applications. Given that all deletion targets are highly connected genes that control key cellular processes, we were concerned that dysregulation major networks may lead to undesirable pleiotropic effects. Since resilience against stress is a complex phenotype, often requiring concerted response from gene networks, we tested the fitness of all single knockout (KO) strains on several stressors (sodium chloride, sodium acetate, furfural, 5-hydroxymethylfurfural, and veratraldehyde) relevant to bioprocessing individually and in combination (FIG. 11B-E). Using parental strain as reference, we observed that performance of strains under stress was highly context-dependent. For example, in sucrose medium, all deletions lost fitness in single or mixed inhibitor cultures (FIGS. 11C and E).

Conversely, on arabinose (with GAL regulon activated), certain KO strains had improved tolerance to stressors (e.g., DTEC1, DMET28) (FIGS. 11D and F). DGLN3 has previously been shown to improve fitness under isobutanol stress²⁷; however, in our study, it was less fit than the parental strain under all stress conditions. Interestingly, it did native substrate supplementation to support biomass generation. However, valorization to high-value products requires efficient catabolism to central metabolic products whose concentrations and fluxes are regulated and associated with growth. Initial studies in literature focused on upstream (“extrinsic”) elements (e.g., heterologous gene expression/activity, transporter engineering, etc.²⁸⁻³²), whereas recent focus has been more “intrinsic”, focusing on functional genomics, adaptively laboratory evolution (ALE), and often network/systems analysis, to identify downstream limitations.⁵⁻¹³ Successes have been abundantly reported with improvements in growth along with a series of deletion and overexpression targets (PHO13, ALD6, ASK10, YPR1, SNF6, demonstrate improved growth rate in a sub-optimal upstream metabolic design in arabinose (FIG. 9E) but lost that benefit in a more optimized design (FIGS. 11C and E). Collectively, these results highlight that deleting genes to remodel expression profiles to enhance a single phenotype (e.g., growth rate on a non-native substrate) can lead to some improvements, but they are often accompanied by negative pleiotropic effects that make the strain less suitable for eventual bioprocessing applications where growth conditions are often non-ideal.

### Discussion

Despite decades of effort engineering synthetic heterotrophy with S. cerevisiae, there is no formalized understanding of what limits the metabolic adaptability of this yeast for growth on pentoses. It is important to differentiate between substrate utilization/uptake from growth since the former can be readily achieved by diverting flux to unwanted or dead-end byproducts (e.g., Crabtree metabolites, organic acids, pentitols) often with
RGT1, CAT8, MSN4, GPD1, CCH1, ADH6, BUD21, ALP1, ISC1, RPL20B, COX4, ISU1, SSK2, YLR042c, CYC8, PHD1,

TEC1, ARR1, etc.^{13,33-39}). Significantly, many targets are unique to individual studies. This is perhaps unsurprising as the impact of such interventions, and even the performance of identical heterologous pathways, can depend on genetic context.^{40,41} Typifying the traditional approach is a recent report where all the aforementioned extensive systems metabolic engineering approaches were used to develop a strain of yeast with high growth aerobic growth rate (0.26 h⁻¹) and short lag phase (9-15 h) on xylose.¹³ Despite this, it is not clear if the insights were translatable to engineering growth on any other substrate.

Through our work, we posit that extensive engineering to identify intrinsic limitations is only required when the initial strain design is sub-optimal. Indeed, constitutive overexpression of upstream metabolic genes results in significant stress¹⁸ and low growth rates that must be compensated for through systems approaches focused on preventing cellular detection of stress rather than activating growth promoting systems. We demonstrated that with an optimal upstream module, strains can attain superior growth profiles (fast specific growth rate, m R 0.24 h⁻¹; short lag phase, <10 h; high final biomass, OD₆₀₀>10) if a growth-associated regulon—the GAL regulon—is activated. This builds on our prior observation that synthetic activation of the GAL regulon potentiates cells for rapid growth on non-native substrates by upregulating growth responsive genes and suppressing starvation responses.¹⁸ Importantly, using this approach our final strain designs had minimal modifications-we deleted only one gene (other than the Leloir pathway genes)—GRE3—to minimize oxidation of substrate pentoses to pentitols.⁴² Many genes that have previously been identified as important inactivation targets to improve growth on pentoses are intact. We also overexpressed only a minimal upstream metabolic module (TAL1 and GAL2²¹) along with the specific pentose metabolic genes (araBAD or XYLA*3-XKSI). Our work strongly argues that growth on pentoses is largely extrinsically controlled, and there are no major intrinsic regulatory or metabolic limitations in this yeast. This insight presents a paradigm shift in engineering synthetic heterotrophy.

An additional advantage of this approach is the preservation of native regulatory systems that are otherwise dysregulated in the traditional engineering approach. We found that while deletion of endogenous genes could improve growth in strains with suboptimal upstream modules under certain conditions, they are associated with pleiotropic defects and are less robust in the presence of growth inhibitors. This is not surprising since most genetic interventions aim to suppress cellular responses to stress rather than promote growth. Thus, strains engineered for synthetic heterotrophy through extensive gene inactivation are less robust for eventual bioprocessing applications. This highlights a major drawback of traditional/systems metabolic engineering approaches like ALE. In contrast, our regulon-based approach is minimalistic and holistic-our strains maintain native regulatory systems and even exploit them toward the engineering goal (i.e., the GAL regulon). On the one hand, our utilization of a single strain background (W303-1a) and catabolic pathway for each substrate in this study could potentially limit the broad applicability of our findings. For example, the use of the oxidoreductase xylose pathway has been demonstrated to improve resistance to lignocellulosic inhibitors relative to the isomerase pathway.⁴³ Also, quantitative measures of strain fitness (i.e., growth rates) are known to be influenced by seemingly confounding factors like the choice of selection marker and the copy number of the plasmid bearing the auxotrophy-complementing gene.⁴⁴ However, the regulon approach we are advocating is not an isolated perturbation to the cell but interfaces with a native regulatory system that coordinates the expression of hundreds of genes. Thus, we believe it is more likely to be transferable to different strain backgrounds and target substrates. Furthermore, this approach significantly simplifies and expedites the design-build-test cycle for synthetic heterotrophy and demonstrates the intrinsic adaptability of yeast toward growth on non-native substrates. We expect that this insight and approach will expand the utility of this yeast for valorizing current and emerging waste and/abundant substrates.

### Methods and Materials

TABLE 4
Reagents and materials used in Example 2

| REAGENT or | | |
|---|---|---|
| RESOURCE | SOURCE | IDENTIFIER |
| Bacterial and virus strains | | |
| Saccharomyces cerevisiae | Euroscarf | BMA64-1a |
| W303-1a | | |
| E. coli E. cloni 10G | Biosearch Technologies | 60107-1 |
| Chemicals, peptides, and | | |
| recombinant proteins | | |
| Yeast Nitrogen Base | RPI | Cat# Y20060-500.0 |
| without amino acids and | | |
| ammonium sulfate | | |
| L(−)-Tryptophan 99% | VWR | Cat# A10230-14 |
| L(+)-Leucine 99% | VWR | Cat# A12311-22 |
| L(+)-Histidine | VWR | Cat# TCH0149-025G |
| Uracil | RPI | Cat# U32000-5.0 |
| Adenine | RPI | Cat# A11500 |
| Salmon sperm DNA | Invitrogen | Cat# 15632011 |
| solution | | |
| Synthetic complement | Sunrise Science products | Cat# 1001-100 |
| mixture | | |
| Veratraldehyde | Sigma | Cat# W310905- |
| | | SAMPLE-K |
| 5-(hydroxymethyl)furfural | Sigma | Cat# W501808-1G-K |
| Furfural | Sigma | Cat# 8040120100 |
| L-(+)-Arabinose | RPI | Cat# A51000 |
| Xylose | Alfa Aesar | Cat# A10643-36 |
| Uracil | RPI | Cat# U32000-5.0 |
| Deposited data | | |
| RNA-sequencing | This study | SRA PRJNA837644 |
| GitHub | | github.com/nair- |
| | | lab/yeast-MINet |
| Experimental models: | | |
| Organisms/strains | | |
| VEG16 (W303-1a | Gopinarayanan et al., 2018 | N/A |
| ΔGAL3 ΔGRE3 ΔGAL1 | | |
| ΔGAL7 ΔGAL10) | | |
| VEG20 (VEG16 GAL2p- | Gopinarayanan et al., 2018 | N/A |
| GAL2^{2.1}-TEF1t::leu2) | | |
| VDT13 (VEG16 GAL^{2.1}- | This study | N/A |
| GAL1p-GAL10p-TAL1- | | |
| Gal3p-Gal3^{Syn4.1}::lys2 | | |
| GAL1p-GAL3^{syn4.1}::leu2 | | |
| VDT27 (VEG16 ADH1t- | This study | N/A |
| GAL2^{2.1} TEF1p/TPI1p- | | |
| TAL1-HXT7t: lys2) | | |
| VDT53 (VDT13 | This study | N/A |
| ΔASH1::KANMX) | | |
| VDT54 (VDT13 | This study | N/A |
| ΔSKO1::KANMX) | | |
| VDT55 (VDT13 | This study | N/A |
| ΔGLN3::KANMX) | | |
| VDT56 (VDT13 | This study | N/A |
| ΔTEC1::KANMX) | | |
| VDT57 (VDT13 | This study | N/A |
| ΔRTG3::KANMX) | | |
| VDT58 (VDT13 | This study | N/A |
| ΔRTG1::KANMX) | | |
| VDT59 (VDT13 | This study | N/A |
| ΔMET28::KANMX) | | |
| VDT60 (VDT13 | This study | N/A |
| ΔMIG2::KANMX) | | |
| Oligonucleotides | | |
| 14-BC-FP (SEQ ID NO: | ACTCACTATAGGGCGA | N/A |
| 9) | ATTGTACTGCAGGTCGA | |
| | CTGGATGGCGGCGTTAG | |
| 38-BC-FP (SEQ ID NO: | ACTCACTATAGGGCGAATTG | N/A |
| 10) | GGACTCCTGTCGACTGG | |
| | ATGGCGGCGTTAG | |
| supp table 2 | | |
| 39-BC-FP (SEQ ID NO: | ACTCACTATAGGGCGAATTG | N/A |
| 11) | TAGGCATGGTCGACTGGAT | |
| | GGCGGCGTTAG | |
| 40-BC-FP (SEQ ID NO: | ACTCACTATAGGGCGAATTG | N/A |
| 12) | TAAGGCGAGTCGACTGGAT | |
| | GGCGGCGTTAG | |
| 41-BC-FP (SEQ ID NO: | ACTCACTATAGGGCGAATTG | N/A |
| 13) | CGTACTAGGTCGACTGG | |
| | ATGGCGGCGTTAG | |
| 42-BC-FP (SEQ ID NO: | ACTCACTATAGGGCGAATT | N/A |
| 14) | GTATCCTCTGTCGACTGGA | |
| | TGGCGGCGTTAG | |
| Recombinant DNA | | |
| pVEG8-WT (pRS426, 2μ | Gopinarayanan et al., 2018 | N/A |
| URA3 ADH1t-EGFP- | | |
| GAL1p/GAL10p- | | |
| KANMX-HXT7t-GAL3p- | | |
| GAL3WT-TEF1t) | | |
| pVEG8-Syn4.1 (pRS426, | Gopinarayanan et al., 2018 | N/A |
| 2μ URA3 ADH1t-EGFP- | | |
| GAL1p/GAL10p- | | |
| KANMX-HXT7t-GAL3p- | | |
| GAL3Syn4.1-TEF1) | | |
| pVEG11 (REG) (pRS426, | Gopinarayanan et al., 2018 | N/A |
| 2μ URA3 ADH1t- | | |
| Piromyces_XYLA″3- | | |
| GAL1p/GAL10p-XKS1- | | |
| HXT7t) | | |
| pVEG15 (CONS) | Gopinarayanan et al., 2018 | N/A |
| (pRS426, 2μ URA3 | | |
| ADH1t- | | |
| Piromyces_XYLA*3- | | |
| TEF1p-TPI1p-XKS1- | | |
| HXT7t) | | |
| pVDT14 (pRS416, CEN | This study | N/A |
| URA3 ADH1t-araB- | | |
| GAL1p/GAL10p-araA- | | |
| HXT7t-GAL7p-araD- | | |
| TEF1t) | | |
| pVDT23 (REG) (pRS426, | This study | N/A |
| 2μ URA3 ADH1t-araB- | | |
| GAL1p/GAL10p-araA- | | |
| HXT7t-GAL7p-araD- | | |
| TEF1t) | | |
| pVDT22 (CONS) | This study | N/A |
| (pRS426, 2μ URA3 | | |
| ADH1t-araB-TEF1p- | | |
| TPI1p-araA-HXT7t- | | |
| GPM1p-araD-TEF1t) | | |
| pVDT29 (pIS385, URA3 | This study | N/A |
| GAL3p-GAL3Syn4.1- | | |
| TEF1t-ADH1t-GAL22.1- | | |
| GAL1/10p-TAL1-HXT7t) | | |
| pVDT30 (plS376, URA3 | This study | N/A |
| GAL1p-GAL3Syn4.1- | | |
| GAL3t) | | |
| pVDT35 (pIS385, URA3 | This study | N/A |
| ADH1t-GAL22.1- | | |
| TEF1p/TPl1p-TAL1- | | |
| Hxt7t) | | |
| pVDT38 (pRS416, CEN | This study | N/A |
| URA3 ADH1t-araA- | | |
| GAL1p/GAL10p-araB- | | |
| HXT7t-GAL7p-araD- | | |
| TEF1t) | | |
| pVDT39 (pRS416, CEN | This study | N/A |
| URA3 ADH1t-araB- | | |
| GAL1p/GAL10p-araD- | | |
| HXT7t-GAL7p-araA- | | |
| TEF1t) | | |
| pVDT40 (pRS416, CEN | This study | N/A |
| URA3 ADH1t-araD- | | |
| GAL1p/GAL10p-araB- | | |
| HXT7t-GAL7p-araA- | | |
| TEF1t) | | |
| pVDT41 (pRS416, CEN | This study | N/A |
| URA3 ADH1t-araD- | | |
| GAL1p/GAL10p-araA- | | |
| HXT7t-GAL7p-araB- | | |
| TEF1t) | | |
| pVDT42 (pRS416, CEN | This study | N/A |
| URA3 ADH1t-araA | | |
| GAL1p/GAL10p-araD- | | |
| HXT7t-GAL7p-araB- | | |
| TEF1t) | | |
| pVDT48 (pRS413, CEN | This study | N/A |
| HIS3 ADH1t- | | |
| Piromyces_XYLA*3- | | |
| GAL1p/GAL10p XKS1- | | |
| HXT7t) | | |
| pVDT49 (pRS413, CEN | This study | N/A |
| HIS3 ADH1t- | | |
| Piromyces_XYLA*3- | | |
| TEF1p-TPl1p-XKS1- | | |
| HXT7t) | | |
| Supp table 3 | | |
| pVDT53 (pRS426, 2u | This study | N/A |
| URA3 GAL1t-GAL1- | | |
| GAL1p/GAL10p- | | |
| GAL10-GAL10t-GAL7p- | | |
| GAL7-GAL7t) | | |
| pVDT54 (pRS423, 2u | This study | N/A |
| HIS3 GAL1t-GAL1- | | |
| GAL1p/GAL10p- | | |
| GAL10-GAL10t-GAL7p- | | |
| GAL7-GAL7t) | | |
| pVDT55 (pRS423, 2u | This study | N/A |
| HIS3 ADH1t- | | |
| Piromyces_XYLA*3- | | |
| GAL1p/GAL10p-XKS1- | | |
| HXT7t) | | |
| pVDT56 (pRS423, 2μ | This study | N/A |
| HIS3 ADH1t-araB- | | |
| GAL1p/GAL10p -araA- | | |
| HXT7t-GAL7p-araD- | | |
| TEF1t) | | |
| pVDT57 (pRS423, 2μ | This study | N/A |
| HIS3 ADH1t-XKS1- | | |
| GAL10p/GAL1p-TEF1t- | | |
| GAL7p- | | |
| Piromyces_XYLA′3- | | |
| HXT7t) | | |
| pVDT58 (pRS423, 2μ | This study | N/A |
| HIS3 ADH1t-XKS1- | | |
| GAL1p/GAL10p-TEF1t- | | |
| GAL7p- | | |
| Piromyces_XYLA*3- | | |
| HXT7t) | | |
| pVDT59 (pRS423, 2μ | This study | N/A |
| HIS3 ADH1t-XKS1- | | |
| GAL1p/GAL10p- | | |
| Piromyces_XYLA*3- | | |
| HXT7t) | | |
| pVDT60 (pRS423, 2u | This study | N/A |
| HIS3 ADH1t-XKS1- | | |
| GAL10p/GAL1p- | | |
| Piromyces_XYLA*3- | | |
| HXT7t) | | |
| pVDT61 (pRS423, 2u | This study | N/A |
| HIS3 ADH1t-XKS1- | | |
| GAL7p-TEF1t- | | |
| GAL1p/GAL10p - | | |
| Piromyces_XYLA″3- | | |
| HXT7t) | | |
| pVDT62 (pRS423, 2μ | This study | N/A |
| HIS3 ADH1t-XKS1- | | |
| GAL7p-TEF1t- | | |
| GAL10p/GAL1p- | | |
| Piromyces_XYLA′3- | | |
| HXT7t) | | |
| Software and algorithms | | |
| Geneious Prime v2022.2.2 | Geneious | RRID: SCR 010519 |
| GraphPad Prism | GraphPad Software Inc. | |
| Microsoft Office Suite | Microsoft, Inc. | |
| Other | | |
| Attune NxT Flow | Invitrogen | N/A |
| cytometer | | |
| Gene Pulser Xcell Total | Bio-Rad | Cat# 1652660 |
| System | | |

### Microbe Strains

S. cerevisiae W303-1a (MATa leu2-3,1 12 trpl-1 canl-100 ura3-1 ade2-1 his3-1 1,15) was obtained from Euroscarf (Oberursel, Germany).

E. coli E. cloni 10G FmcrA D(mrr-hsdRMS-mcrBC) endAl recAl F80dlacZDM15 DlacX74 araD139 D(ara,leu)7697galU galK rpsL nupG itonA (StrR) was obtained from New England Biolabs (Ipswich, MA)

### Strains and Plasmids

Strain W303-1a (MATa leu2-3,112 trpl-1 canl-100 ura3-1 ade2-1 his3-11,15) and plasmids pIS374, pIS376, pIS385 were obtained from Euroscarf (Oberursel, Germany). The plasmids were constructed in the present using NEB-HiFi DNA assembly master mix from NEB (Ipswich, MA).

### Growth Studies

Overnight inoculums were grown in the required dropout SC medium (6.7 g/L yeast nitrogen base without amino acids, 2 g/L dropout mix) with sucrose (2%). The culture was washed twice in the growth medium and resuspended at an initial OD600 of 0.1 with appropriate sugar (2%) in 250 mL shake flasks containing 20 mL of media. OD600 measurement was checked at frequent time intervals (3-6 h) on SpectraMax M3 spectrophotometer (Molecular Devices). Growth rate was determined by plotting the values in GraphPad Prism following non-linear regression and using exponential growth equation, Y=Y0 exp(kX).

### Directed Evolution

Error prone PCR libraries of arabinose cassettes (pVDT14, pVDT38-42) were generated as described earlier.18 Six-barcoded primers (Table S4) were used to track the proportion of the lineage during the enrichment. The amplicons from error prone PCR were assembled into pRS413 plasmids using yeast gap-repair cloning. To attain high library size, the linear fragments were transformed into yeast by electroporation as described previously.45 We attained library size of ˜108 CFUs which was then subjected to enrichment on arabinose in 2YP (20 g/L yeast extract, 40 g/L peptone, 100 mg/L adenine hemisulfate) as well SC media. The cell pellets from each passage were frozen for quantifying barcodes using amplicon sequencing (Genewiz, Cambridge, MA). At the end of enrichment, the library pool was plated on 2YP+Ara and SC+Ara and 18 colonies were randomly picked for growth rate determination. The plasmids from best variants were isolated from strain, transformed into E. coli. The corresponding plasmid isolated from E. coli was sequenced and re-transformed into REG-strain for growth rate determination.

### Genomics Integrations

Accessory cassettes (pVDT29, 30, 35) were integrated into VEG16 (W303-1a, DGRE3, DGAL1, DGAL3, DGAL7, DGAL10)18 strain via disintegrator plasmid.46 Counterselection was performed on SC medium with 1 g/L 5-FOA to remove the URA3 marker and the locus was amplified, and sequence confirmed.

### Deletion Library

To generate the deletion library, the cassettes for the required targets were amplified from the genomic DNA of the appropriate strains from the KO collection.47 Since the cassettes contained KANMX marker, the library was selected on YP+G418 (400 mg/mL). The pool was stored as a stock and used for studying fitness by transforming it with plasmids for galactose, xylose, and arabinose utilizing cassettes.

qPCR Expression Analysis

Total yeast RNA isolation was performed on OD ˜1 of yeast cells collected from mid-log phase growth using the GeneJet RNA Purification Kit (Thermofisher Scientific Catalog #: K0731) according to the instructions for isolating yeast RNA. Two samples were collected for each growth condition. The concentration of total RNA isolated was obtained by measuring 10-fold dilutions from each sample on a spectrophotometer. Using these concentrations, 2.5 mg of total RNA was subjected to the ‘routine’ DNase treatment protocol using the Invitrogen Turbo DNA-free Kit (Thermofisher Scientific Catalog #: AM1907). Next, 20% of the final reaction (10-50 mL, targeting 500 ng RNA) was used to synthesize cDNA for the sample using the Invitrogen SuperScript IV First-Strand Synthesis System (Thermofisher Scientific Catalog #: 18091050). The random hexamers supplied by the kit were used to prime the reverse transcriptase enzyme. cDNA samples were diluted 100-fold (2 mL into 198 mL dH2O, of which 2 mL was used as the template for a 15 mL qPCR reaction using the Applied Biosystems PowerUp SYBR Green Master Mix (Thermofisher Scientific Catalog #: A25741). Each sample was tested for the expression level of five genes: the three arabinose catabolic genes (araA, araB, and araD) as well as two housekeeping genes (TFC1 and UBC6). Reactions were run on an Applied Biosystems Quantstudio 5 Real-Time PCR Instrument. The relative expression level of each catabolic genes was calculated by subtracting the geometric mean of the cycle thresholds (Ct) of the two housekeeping genes from the Ct of that catabolic gene, followed by conversion of the log-base two Ct into a non-log value that can be compared across genes.

### RNA-Seq

Transcriptomics of strains WT, XYL-REG, ARA-REG were performed on the mid-log phase cultures grown on their respective carbon source (galactose, xylose, or arabinose). Cells pellets were washed twice in water and stored at −80° C. and outsourced to Genewiz Inc. for RNA extraction and sequencing. RNA-seq was performed on Illumina HiSeq. Raw FASTQ files were processed for differential expression analysis using GeneiousPrime. The possible adapter sequences and low-quality short-read (less than 50 bp) trimming were performed using BB-Trim package. The reads were aligned to the reference genome W303 obtained from Saccharomyces Genome Database using Bow-Tie2 package. The edgeR package was used to normalize the gene count based on library size and was converted to cpm (counts per million) using. DESEQ2 package was used for differential gene expression analysis. Genes with p-values <0.05 and fold change of R 2 were considered as differentially expressed.

### Amplicon Sequencing Analysis

The total DNA was extracted and quantified using a spectrophotometer and approximately 100 ng was used as template for PCRs. Unique, barcoded primers flanked by Illumina sequencing adaptors were used to generate the amplicons after 15-20 cycles of PCR for sequencing. The barcoded samples were pooled and sent for amplicon sequencing (23250 bp, Genewiz, New Jersey, USA). For each pooled sample, we received approximately 100,000 sequencing reads. These data were processed according to a previously described bioinformatic workflow using Geneious Primer 2020.2.4.48 Briefly, the reads were paired and merged using the BBMerge package and filtered for poor-quality reads using the BBDuk package. The reads were mapped using BowTie2 and gene expression was calculated and differential expression was determined using DESeq2.49

### Network Analysis

Our analysis utilizes three yeast genetic regulatory networks (EGRIN, YEASTRACT, and CLR)24 in the context of integrating regulatory and metabolic networks to enable more accurate prediction of yeast phenotypes in different growth conditions.50 The Environment and Gene Regulatory Influence Network (EGRIN) consists of 92 regulators and 2,588 interactions and was constructed using the cMonkey and Inferelator computational tools trained on expression data from 2,929 microarray experiments to identify gene clusters and potential regulatory genes. The YEASTRACT gene regulatory network was extracted directly from the YEASTRACT database2 and consists of 177 regulators and 31,075 regulatory associations based on both ‘direct evidence’ chromatin immunoprecipitation (ChIP), ChIP-on-chip, electrophoretic mobility shift assay, or examination of the effect of TF binding site mutations on target gene expression as well as ‘indirect evidence’ provided by gene expression changes in response to deletion, mutation, or overexpression of a given TF3.

The EGRIN, YEASTRACT, and CLR yeast gene regulatory networks (GRN) were downloaded as Microsoft Excel files from the supplementary information of a recent study by Wang et al.²⁴ These excel files captured the GRN as two-column lists of genes with each row representing an inferred connection between the genes. We imported these files as networks in the open-source network visualization and analysis software Cytoscape.25 For each network, we created spreadsheets that associates the fold-change in expression and corrected p-value from the differential gene expression analysis between the pentose (REG) and galactose grown strains with each unique yeast gene found in that network. By importing these files into Cytoscape, we were able to map this additional information onto the nodes in the networks. Next, each network was filtered by removing all nodes corresponding to genes with Benjamini-Hochberg corrected p-values >0.05. The “Analyze Network” function was used to generate betweenness centrality (BC), a quantitative measure of the centrality of a given node in a network, values for each of the remaining nodes. The final list of 24 deletion targets was manually curated from a combined list of the top 15 genes by BC from each of the three networks based on which were most likely to be directly related to the metabolism of pentoses. The R programming language was used for importing and manipulating data.

### Stressor Studies

We determined fitness of the strains in presence of various stressors encountered during biomanufacturing using plate assay. Briefly, we tested the growth of by performing spot dilution on 2YP supplemented with 2% sucrose or arabinose and various concentration of the stressors. The plates after incubation were imaged and the colonies were counted to determine the CFUs. The CFUs in the presence of the stressor was divided by the CFUs in absence of the stressor to determine the fitness of that strain. This value was normalized to the fitness of the parental strain and was expressed as the relative fitness.

### Quantification and Statistical Analysis

All cell growth assays and qPCRs were done in triplicates (or more replicates) as indicated in the figure legends, and the data are presented as mean±SD or SEM. All data fitting and statistical analysis was performed using GraphPad Prism, or MS-Excel software.

### REFERENCES

1. Sun, J., Sun, W., Zhang, G., Lv, B., and Li, C. (2022). High Efficient Production of Plant Flavonoids by Microbial Cell Factories: Challenges and Opportunities (Metabolic Engineering).
2. Franden, M. A., Jayakody, L. N., Li, W. J., Wagner, N. J., Cleveland, N. S., Michener, W. E., Hauer, B., Blank, L. M., Wierckx, N., Klebensberger, J., and Beckham, G. T. (2018). Engineering Pseudomonas putida KT2440 for efficient ethylene glycol utilization. Metab. Eng. 48, 197-207.
3. Pontrelli, S., Chiu, T. Y., Lan, E. I., Chen, F. Y. H., Chang, P., and Liao, J. C. (2018). Escherichia coli as a host for metabolic engineering. Metab. Eng. 50, 16-46.
4. Sun, T., Yu, Y., Wang, K., Ledesma-Amaro, R., and Ji, X. J. (2021). Engineering Yarrowia lipolytica to produce fuels and chemicals from xylose: A review. Bioresour. Technol. 337, 125484.
5. Hanly, T. J., and Henson, M. A. (2011). Dynamic flux balance modeling of microbial co-cultures for efficient batch fermentation of glucose and xylose mixtures. Biotechnol. Bioeng. 108, 376-385.
6. Lee, S. M., Jellison, T., and Alper, H. S. (2012). Directed evolution of xylose isomerase for improved xylose catabolism and fermentation in the yeast Saccharomyces cerevisiae. Appl. Environ. Microbiol. 78, 5708-5716.
7. Diao, L., Liu, Y., Qian, F., Yang, J., Jiang, Y., and Yang, S. (2013). Construction of fast xylose-fermenting yeast based on industrial ethanol-producing diploid Saccharomyces cerevisiae by rational design and adaptive evolution. BMC Biotechnol. 13, 110-119.
8. Wohlbach, D. J., Kuo, A., Sato, T. K., Potts, K. M., Salamov, A. A., LaButti, K. M., Sun, H., Clum, A., Pangilinan, J. L., Lindquist, E. A., et al. (2011). Comparative genomics of xylose-fermenting fungi for enhanced biofuel production. Proc. Natl. Acad. Sci. USA 108, 13212-13217.
9. Matsushika, A., Inoue, H., Kodaki, T., and Sawayama, S. (2009). Ethanol production from xylose in engineered Saccharomyces cerevisiae strains: current state and perspectives. Appl. Microbiol. Biotechnol. 84, 37-53.
10. Lee, M., Rozeboom, H. J., Keuning, E., de Waal, P., and Janssen, D. B. (2020). Structure-based directed evolution improves S. cerevisiae growth on xylose by influencing in vivo enzyme performance. Biotechnol. Biofuels 13, 5-18.
11. Dai, Z., and Nielsen, J. (2015). Advancing metabolic engineering through systems biology of industrial microorganisms. Curr. Opin. Biotechnol. 36, 8-15.
12. Presnell, K. V., and Alper, H. S. (2019). Systems metabolic engineering meets machine learning: A new era for data-driven metabolic engineering. Biotechnol. J. 14, 1800416.
13. Li, J., Guo, Y., Shi, K., Li, X., Xie, J., Chen, Y., and Nielsen, J. (2021). Metabolic network remodelling enhances yeast's fitness on xylose using aerobic glycolysis. Int. J. Psychol. 56, 783-790.
14. Sun, L., and Jin, Y. S. (2021). Xylose assimilation for the efficient production of biofuels and chemicals by engineered Saccharomyces cerevisiae. Biotechnol. J. 16, 2000142.
15. Wang, X., Yang, J., Yang, S., and Jiang, Y. (2019). Unraveling the genetic basis of fast 1-arabinose consumption on top of recombinant xylo- se-fermenting Saccharomyces cerevisiae. Biotechnol. Bioeng. 116, 283-293.
16. Garcia Sanchez, R., Karhumaa, K., Fonseca, C., Sanchez Nogue, V., Almeida, J. R., Larsson, C. U., Bengtsson, O., Bettiga, M., Hahn- Hgerdal, B., and Gorwa-Grauslund, M. F. (2010). Improved xylose and arabinose utilization by an industrial recombinant Saccharomyces cerevisiae strain using evolutionary engineering. Biotechnol. Biofuels 3, 13.
17. Wisselink, H. W., Toirkens, M. J., Wu, Q., Pronk, J. T., and van Maris, A. J. A. (2009). Novel evolutionary engineering approach for accelerated utilization of glucose, xylose, and arabinose mixtures by engineered Saccharomyces cerevisiae strains. Appl. Environ. Microbiol. 75, 907-914.
18. Endalur Gopinarayanan, V., and Nair, N. U. (2018). A semi-synthetic regulon enables rapid growth of yeast on xylose. Nat. Commun. 9, 1233.
19. Wisselink, H. W., Toirkens, M. J., del Rosario Franco Berriel, M., Winkler, A. A., van Dijken, J. P., Pronk, J. T., and van Maris, A. J. A. (2007). Engineering of Saccharomyces cerevisiae for efficient anaerobic alcoholic fermentation of L-arabinose. Appl. Environ. Microbiol. 73, 4881-4891.
20. Deng, J., Wu, Y., Zheng, Z., Chen, N., Luo, X., Tang, H., and Keasling, J. D. (2021). A synthetic promoter system for well-controlled protein expression with different carbon sources in Saccharomyces cerevisiae. Microb. Cell Fact. 20, 202-210.
21. Peng, B., Williams, T. C., Henry, M., Nielsen, L. K., and Vickers, C. E. (2015). Controlling heterologous gene expression in yeast cell factories on different carbon substrates and across the diauxic shift: a comparison of yeast promoter activities. Microb. Cell Fact. 14, 91.
22. Sullivan, S. F., Shetty, A., Bharadwaj, T., Krishna, N., Trivedi, V. D., Endalur Gopinarayanan, V., Sellers, D. M., Kumar, R. P., and Nair, N. U. (2022). Towards universal synthetic heterotrophy using a metabolic coordinator. Preprint at bioRxiv.
23. Dai, Z., Huang, M., Chen, Y., Siewers, V., and Nielsen, J. (2018). Global re- wiring of cellular metabolism renders Saccharomyces cerevisiae Crabtree negative. Nat. Commun. 9, 3059.
24. Wang, Z., Danziger, S. A., Heavner, B. D., Ma, S., Smith, J. J., Li, S., Herricks, T., Simeonidis, E., Baliga, N. S., Aitchison, J. D., and Price, N. D. (2017). Combining inferred regulatory and reconstructed metabolic net- works enhances phenotype prediction in yeast. PLoS Comput. Biol. 13, e1005489.
25. Shannon, P. (2003). Markiel a, Ozier 0, et al. cytoscape: a software environment for integrated models of biomolecular interaction networks. Genome Res. 13, 1.
26. Costa, C. E., Carvalho, P., and Domingues, L. (2022). Strategic combination of different promoters in lactose metabolisation and host chassis se- lection for high bioethanol titres from dairy wastes. Bioresource Technology Reports 19, 101131.
27. Kuroda, K., Hammer, S. K., Watanabe, Y., Montano Lopez, J., Fink, G. R., Stephanopoulos, G., Ueda, M., and Avalos, J. L. (2019). Critical roles of the pentose phosphate pathway and GLN3 in isobutanol-specific toler- ance in yeast. Cell Syst. 9, 534-547.e5.
28. Young, E., Lee, S. M., and Alper, H. (2010). Optimizing pentose utilization in yeast: the need for novel tools and approaches. Biotechnol. Biofuels 3, 24.
29. Young, E. M., Comer, A. D., Huang, H., and Alper, H. S. (2012). A molecular transporter engineering approach to improving xylose catabolism in Saccharomyces cerevisiae. Metab. Eng. 14, 401-411.
30. Nijland, J. G., and Driessen, A. J. M. (2019). Engineering of pentose trans- port in Saccharomyces cerevisiae for biotechnological applications. Front. Bioeng. Biotechnol. 7, 464.
31. Wiedemann, B., and Boles, E. (2008). Codon-optimized bacterial genes improve L-arabinose fermentation in recombinant Saccharomyces cerevisiae. Appl. Environ. Microbiol. 74, 2043-2050.
32. Endalur Gopinarayanan, V., and Nair, N. U. (2019). Pentose metabolism in Saccharomyces cerevisiae: the need to engineer global regulatory systems. Biotechnol. J. 14, 1800364.
33. Kim, S. R., Skerker, J. M., Kang, W., Lesmana, A., Wei, N., Arkin, A. P., and Jin, Y. S. (2013). Rational and evolutionary engineering approaches un- cover a small set of genetic changes efficient for rapid xylose fermentation in Saccharomyces cerevisiae. PLoS One 8, e57048.
34. Usher, J., Balderas-Hernandez, V., Quon, P., Gold, N. D., Martin, V. J., Mahadevan, R., and Baetz, K. (2011). Chemical and Synthetic Genetic Array Analysis Identifies Genes that Suppress Xylose Utilization. G3 (Bethesda) 1, 247-258.
35. Shen, Y., Chen, X., Peng, B., Chen, L., Hou, J., and Bao, X. (2012). An efficient xylose-fermenting recombinant Saccharomyces cerevisiae strain obtained through adaptive evolution and its global transcription profile. Appl. Microbiol. Biotechnol. 96, 1079-1091.
36. Dos Santos, L. V., Carazzolle, M. F., Nagamatsu, S. T., Sampaio, N. M. V., Almeida, L. D., Pirolla, R. A. S., Borelli, G., Corre\hat{ }a, T. L. R., Argueso, J. L., and Pereira, G. A. G. (2016). Unraveling the genetic basis of xylose consumption in engineered Saccharomyces cerevisiae strains. Sci. Rep. 6, 38676.
37. Parachin, N. S., Bengtsson, O., Hahn-Hgerdal, B., and Gorwa-Grauslund, M. F. (2010). The deletion of YLR042c improves ethanolic xylose fermentation by recombinant Saccharomyces cerevisiae. Yeast 27, 741-751.
38. Zha, J., Shen, M., Hu, M., Song, H., and Yuan, Y. (2014). Enhanced expression of genes involved in initial xylose metabolism and the oxidative pentose phosphate pathway in the improved xylose-utilizing Saccharomyces cerevisiae through evolutionary engineering. J. Ind. Microbiol. Biotechnol. 41, 27-39.
39. Wahlbom, C. F., Cordero Otero, R. R., van Zyl, W. H., Hahn-Hgerdal, B., and Jonsson, L. J. (2003). Molecular analysis of a Saccharomyces cerevisiae mutant with improved ability to utilize xylose shows enhanced expression of proteins involved in transport, initial xylose metabolism, and the pentose phosphate pathway. Appl. Environ. Microbiol. 69, 740-746.
40. Cunha, J. T., Aguiar, T. Q., Romaní, A., Oliveira, C., and Domingues, L. (2015). Contribution of PRS3, RPB4 and ZWF1 to the resistance of indus- trial Saccharomyces cerevisiae CCUG53310 and PE-2 strains to lignocellulosic hydrolysate-derived inhibitors. Bioresour. Technol. 191, 7-16.
41. Romani, A., Pereira, F., Johansson, B., and Domingues, L. (2015). Metabolic engineering of Saccharomyces cerevisiae ethanol strains PE-2 and CAT-1 for efficient lignocellulosic fermentation. Bioresour. Technol. 179, 150-158.
42. Traff, K. L., Otero Cordero, R. R., van Zyl, W. H., and Hahn-Hgerdal, B. (2001). Deletion of the GRE3 aldose reductase gene and its influence on xylose metabolism in recombinant strains of Saccharomyces cerevisiae expressing the xylA and XKS1 genes. Appl. Environ. Microbiol. 67, 5668-5674.
43. Cunha, J. T., Soares, P. O., Romaní, A., Thevelein, J. M., and Domingues, L. (2019). Xylose fermentation efficiency of industrial Saccharomyces cerevi- siae yeast with separate or combined xylose reductase/xylitol dehydrogenase and xylose isomerase pathways. Biotechnol. Biofuels 12, 1-14.
44. Pronk, J. T. (2002). Auxotrophic yeast strains in fundamental and applied research. Appl. Environ. Microbiol. 68, 2095-2100.
45. Thompson, J. R., Register, E., Curotto, J., Kurtz, M., and Kelly, R. (1998). An improved protocol for the preparation of yeast cells for transformation by electroporation. Yeast 14, 565-571.
46. Sadowski, I., Su, T. C., and Parent, J. (2007). Disintegrator vectors for sin- gle-copy yeast chromosomal integration. Yeast 24, 447-455.
47. Giaever, G., Chu, A. M., Ni, L., Connelly, C., Riles, L., Veronneau, S., Dow, S., Lucau-Danila, A., Anderson, K., Andre, B., et al. (2002). Functional profiling of the Saccharomyces cerevisiae genome. nature 418, 387-391.
48. Trivedi, V. D., Chappell, T. C., Krishna, N. B., Shetty, A., Sigamani, G. G., Mohan, K., Ramesh, A., Nair, N. U., and Nair, N. U. (2022). In-Depth Sequence-Function Characterization Reveals Multiple Pathways to Enhance Enzymatic Activity. ACS Catal. 12, 2381-2396.
49. Love, M. I., Huber, W., and Anders, S. (2014). Moderated estimation of fold change and dispersion for RNA-seq data with DESeq2. Genome Biol. 15, 550.
50. Herrgard, M. J., Lee, B. S., Portnoy, V., and Palsson, B. Ø. (2006). Integrated analysis of regulatory and metabolic networks reveals novel regulatory mechanisms in Saccharomyces cerevisiae. Genome Res. 16, 627-635.

### Additional Embodiments

Provided here is a non-exclusive list of additional embodiments of the present disclosure:

Embodiment 1. An engineered protein, wherein the engineered protein is a variant of Gal3p, wherein the variant of Gal3p is fully activated.

Embodiment 2. The engineered protein of embodiment 1, wherein the variant of Gal3p possesses a conformational change corresponding to galactose-bound Gal3p.

Embodiment 3. The engineered protein of any one of the preceding embodiments, wherein the variant of Gal3p comprises SEQ ID NO: 1 or a sequence having at least 80% identity thereto.

Embodiment 4. The engineered protein of any one of the preceding embodiments, wherein the variant of Gal3p is Gal3pMC

Embodiment 5. The engineered protein of any one of the preceding embodiments, wherein the engineered protein activates the galactose regulon.

Embodiment 6. The engineered protein of embodiment 5, wherein the galactose regulon is activated by indirect action.

Embodiment 7. The engineered protein any one of the preceding embodiments, wherein the engineered protein allows a microbial cell comprising or expressing the engineered protein to grow on a non-native substrate.

Embodiment 8. A nucleic acid construct encoding the engineered protein of any one of the preceding embodiments.

Embodiment 9. The nucleic acid construct of embodiment 8, wherein the nucleic acid construct comprises SEQ ID NO: 15 or a sequence having at least 80% identity thereto.

Embodiment 10. A microbial cell comprising or expressing the engineered protein of any one of embodiments 1-7 and/or the nucleic acid construct of embodiment 8 or 9.

Embodiment 11. A multicellular microbial organism comprising at least one microbial cell of embodiment 10.

Embodiment 12. The microbial cell of embodiment 10, wherein the microbial cell is a yeast.

Embodiment 13. The microbial cell of embodiment 12, wherein the yeast is Saccharomyces cerevisiae.

Embodiment 14. The microbial cell of any one of embodiments 11-13, wherein the microbial cell grows in an inducer-independent manner.

Embodiment 15. A method of engineering a microbial organism for growth on a non-native substrate, wherein the method comprises synthetically activating the GAL response system in the microbial organism.

Embodiment 16. The method of embodiment 15, wherein a semi-synthetic GAL regulon activates the GAL response system.

Embodiment 17. The method of embodiment 16, further comprising synergizing activation of the semi-synthetic GAL regulon with an optimized upstream heterologous metabolic module.

Embodiment 18. The method of embodiment 18, wherein the optimized upstream heterologous metabolic module comprises a deletion of at least one negative effector gene.

Embodiment 18. The method of embodiment 18, wherein the at least one negative effector gene minimizes oxidation of substrate pentoses to pentitols.

Embodiment 20. The method of embodiment 19, wherein the at least one negative effector gene is GRE3.

Embodiment 21. The method of any one of embodiments 17-20, wherein the optimized upstream heterologous metabolic module comprises an overexpression of at least one positive effector gene.

Embodiment 22. The method of embodiment 21, wherein the at least one positive effector gene is a pentose metabolic gene.

Embodiment 23. The method of embodiment 21, where the at least one positive effector gene is selected from the group consisting of TAL1, GAL2, araBAD, and XYLA*3-XKS1.

Embodiment 24. The method of embodiment 15 or 16, wherein the method does not comprise modifying catabolic genes in the microbial organism.

Embodiment 25. A method of engineering a microbial organism for growth on a non-native substrate, wherein the method consists of synthetically activating the GAL response system in the microbial organism.

Embodiment 26. A method of growing a microbial organism on a non-native substrate, wherein the method comprises expressing the engineered protein of any one of embodiments 1-7 or the nucleic acid construct of embodiment 8 or 9 in the microbial organism.

Embodiment 27. The method of embodiment 26, wherein the microbial organism grows in an inducer-independent manner.

Embodiment 28. The method of any one of embodiments 15-27, wherein the non-native substrate is a sugar.

Embodiment 29. The method of any one of embodiments 15-28, wherein the sugar is selected from the group consisting of arabinose, xylose, cellobiose, and raffinose.

Embodiment 30. The method of 28, wherein the sugar excludes at least one of glucose and sucrose.

Embodiment 31. The method of any one of embodiments 15-30, wherein the microbial organism is a yeast.

Embodiment 32. The method of embodiment 31, wherein the yeast is Saccharomyces cerevisiae.

Embodiment 33. A method of engineering a microbial organism for growth on a non-native substrate, wherein the method comprises expressing an engineered protein in the microbial organism, wherein the engineered protein is Gal3pMC, wherein the microbial organism is Saccharomyces cerevisiae, and wherein the non-native substrate is selected from the group consisting of arabinose, xylose, cellobiose, and raffinose.

Embodiment 34. The method of embodiment 33, further comprising synergizing activation of the semi-synthetic GAL regulon with an optimized upstream heterologous metabolic module.

Embodiment 35. The method of embodiment 34, wherein the optimized upstream heterologous metabolic module comprises a deletion of GRE3 and/or an overexpression of at least one positive effector gene selected from the group consisting of TAL1, GAL2, araBAD, and XYLA*3-XKS1.

```
SEQUENCES
Gal3p^{MC}
SEQ ID NO: 1
MNTNVPIFSSPVRDLPRSFEQKHLAVVDAFFQTYHVKPDFIARSP
GRVNLIGEHIDYCDFSVLPLAINMDMLCAVKILDEKNPSITLTNA
DPKFAQRKFDLPLDGSYMVIDPSVSEWSNYFKCGLHVAHSYLKKI
APERFNNTPLVGAQIFCQSDIPTGGGLSSAFTCAAALATIRANMG
KNFDISKKDLTRITAVAEHYVGVNNGGMDQATSVYGEEDHALYVE
FRPKLKATPFKYPQLKNHEISFVIANTLVKSNKFETAPTNYNLRV
LEVTVAANALATRYSVALPSHKDNSNSERGNLRDFMDAYYARYEN
QAQPWNGDIGTGIERLLKMLQLVEESFSRKKSGFTVHEASTALNC
SREEFTRDYLTTFPVRFQVLKLYQRAKHVYSESLRVLKALKMMTS
ATFHTDEDFFTDFGRLMNESQASCDKLYECSCIETNQICSIALAN
GSFGSRLTGAGWGGCTIHLVPSGANGNVEQVRKALIEKFYNVRYP
DLTDEELKDAIIVSKPALGTCLYEQ
Gal3p^{WT}
SEQ ID NO: 2
MNTNVPIFSSPVRDLPRSFEQKHLAVVDAFFQTYHVKPDFIARSP
GRVNLIGEHIDYCDFSVLPLAIDVDMLCAVKILDEKNPSITLTNA
DPKFAQRKFDLPLDGSYMAIDPSVSEWSNYFKCGLHVAHSYLKKI
APERFNNTPLVGAQIFCQSDIPTGGGLSSAFTCAAALATIRANMG
KNFDISKKDLTRITAVAEHYVGVNNGGMDQATSVYGEEDHALYVE
FRPKLKATPFKFPQLKNHEISFVIANTLVKSNKFETAPTNYNLRV
IEVTVAANALATRYSVALPSHKDNSNSERGNLRDFMDAYYARYEN
QAQPWNGDIGTGIERLLKMLQLVEESFSRKKSGFTVHEASTALNC
SREEFTRDYLTTFPVRFQVLKLYQRAKHVYSESLRVLKALKMMTS
ATFHTDEDFFTDFGRLMNESQASCDKLYECSCIETNQICSIALAN
GSFGSRLTGAGWGGCTIHLVPSGANGNVEQVRKALIEKFYNVRYP
DLTDEELKDAIIVSKPALGTCLYEQ
Gal3p^{Syn4.1}
SEQ ID NO: 3
MNTNVPIFSSPVRDLPRSFEQKHLAVVDAFFQTYHVKPDFIARSP
GRVNLIGEHIDYCDFSVLPLAINMDMLCAVKILDEKNPSITLTNA
DPKFAQRKFDLPLDGSYMVIDPSVSEWSNYFKCGLHVAHSYLKKI
APERFNNTPLVGAQIFCQSDIPTGGGLSSAFTCAAALATIRANMG
KNFDISKKDLTRITAVAEHYVGVNNGGMDQATSVYGEEDHALYVE
FRPKLKATPFKFPQLKNHEISFVIANTLVKSNKFETAPTNYNLRV
LEVTVAANALATRYSVALPSHKDNSNSERGNLRDFMDAYYARYEN
QAQPWNGDIGTGIERLLKMLQLVEESFSRKKSGFTVHEASTALNC
SREEFTRDYLTTFPVRFQVLKLYQRAKHVYSESLRVLKALKMMTS
ATFHTDEDFFTDFGRLMNESQASCDKLYECSCIETNQICSIALAN
GSFGSRLTGAGWGGCTIHLVPSGANGNVEQVRKALIEKFYNVRYP
DLTDEELKDAIIVSKPALGTCLYEQ
Gal3p^{S509P}
SEQ ID NO: 4
MNTNVPIFSSPVRDLPRSFEQKHLAVVDAFFQTYHVKPDFIARSP
GRVNLIGEHIDYCDFSVLPLAIDVDMLCAVKILDEKNPSITLTNA
DPKFAQRKFDLPLDGSYMAIDPSVSEWSNYFKCGLHVAHSYLKKI
APERFNNTPLVGAQIFCQSDIPTGGGLSSAFTCAAALATIRANMG
KNFDISKKDLTRITAVAEHYVGVNNGGMDQATSVYGEEDHALYVE
FRPKLKATPFKFPQLKNHEISFVIANTLVKSNKFETAPTNYNLRV
IEVTVAANALATRYSVALPSHKDNSNSERGNLRDFMDAYYARYEN
QAQPWNGDIGTGIERLLKMLQLVEESFSRKKSGFTVHEASTALNC
SREEFTRDYLTTFPVRFQVLKLYQRAKHVYSESLRVLKALKMMTS
ATFHTDEDFFTDFGRLMNESQASCDKLYECSCIETNQICSIALAN
GSFGSRLTGAGWGGCTIHLVPSGANGNVEQVRKALIEKFYNVRYP
DLTDEELKDAIIVPKPALGTCLYEQ
Gal3p^{F237Y}
SEQ ID NO: 5
MNTNVPIFSSPVRDLPRSFEQKHLAVVDAFFQTYHVKPDFIARSP
GRVNLIGEHIDYCDFSVLPLAIDVDMLCAVKILDEKNPSITLTNA
DPKFAQRKFDLPLDGSYMAIDPSVSEWSNYFKCGLHVAHSYLKKI
APERFNNTPLVGAQIFCQSDIPTGGGLSSAFTCAAALATIRANMG
KNFDISKKDLTRITAVAEHYVGVNNGGMDQATSVYGEEDHALYVE
FRPKLKATPFKYPQLKNHEISFVIANTLVKSNKFETAPTNYNLRV
IEVTVAANALATRYSVALPSHKDNSNSERGNLRDFMDAYYARYEN
QAQPWNGDIGTGIERLLKMLQLVEESFSRKKSGFTVHEASTALNC
SREEFTRDYLTTFPVRFQVLKLYQRAKHVYSESLRVLKALKMMTS
ATFHTDEDFFTDFGRLMNESQASCDKLYECSCIETNQICSIALAN
GSFGSRLTGAGWGGCTIHLVPSGANGNVEQVRKALIEKFYNVRYP
DLTDEELKDAIIVSKPALGTCLYEQ
Gal3p^{F237Y + S509P}
SEQ ID NO: 6
MNTNVPIFSSPVRDLPRSFEQKHLAVVDAFFQTYHVKPDFIARSP
GRVNLIGEHIDYCDFSVLPLAIDVDMLCAVKILDEKNPSITLTNA
DPKFAQRKFDLPLDGSYMAIDPSVSEWSNYFKCGLHVAHSYLKKI
APERFNNTPLVGAQIFCQSDIPTGGGLSSAFTCAAALATIRANMG
KNFDISKKDLTRITAVAEHYVGVNNGGMDQATSVYGEEDHALYVE
FRPKLKATPFKYPQLKNHEISFVIANTLVKSNKFETAPTNYNLRV
IEVTVAANALATRYSVALPSHKDNSNSERGNLRDFMDAYYARYEN
QAQPWNGDIGTGIERLLKMLQLVEESFSRKKSGFTVHEASTALNC
SREEFTRDYLTTFPVRFQVLKLYQRAKHVYSESLRVLKALKMMTS
ATFHTDEDFFTDFGRLMNESQASCDKLYECSCIETNQICSIALAN
GSFGSRLTGAGWGGCTIHLVPSGANGNVEQVRKALIEKFYNVRYP
DLTDEELKDAIIVPKPALGTCLYEQ
Modeled Gal3p
SEQ ID NO: 7
SNTNVPIFSSPVRDLPRSFEQKHLAVVDAFFQTYHVKPDFIARSP
GRVNLIGEHIDYCDFSVLPLAIDVDMLCAVKILDEKNPSITLTNA
DPKFAAQRKFDLPLDGSYMAIDPSVSEWSNYFXCGLHVAHSYLKK
IAPERFNNTPLVGAQIFCQSDIPTGGGLSSAFTCAAALATIRANM
GXNFDISKKDLTRITAVAEHYVGVNNGGMDQATSVYGEEDHALYV
EFRPKLKATPFKFPQLKNHEISFVIANTLVKSNKFETAPTNYNLR
VIEVTVAANALATRYSVALPSHKDNSNSERGNLRDFMDAYYARYE
NQAQPWNGDIGTGIERLLKMLQLLVEESFSRKKSGFTVHEASTAL
NCSREEFTRDYLTTFPVRFQVLKLYQRAKHVYSESLRVLKALKMM
TSATFHTDEDFFTDFGRLMNESQASCDKLYECSCIETNQICSIAL
ANGSFGSRLTGAGWGGCTIHLVPSGANGNVEQVRKALIEKFYNVR
YPDLTDEKLKDAIVSXPALGTCLYEQ
3V2U
SEQ ID NO: 8
NTNVPIFSSPRSFEQKHLAVVDAFFQTYHVKPDFIARSPGRVNLI
GEHIDYCDFSVLPLAIDVDMLCAVKILDEKNPSITLTNADPKFAA
QRKFDLPLDGSYMAIDPSVSEWSNYFXCGLHVAHSYLKKIAPERF
NNTPLVGAQIFCQSDIPTGGGLSSAFTCAAALATIRANMGXNFDI
SKKDLTRITAVAEHYVGVNNGGMDQATSVYGEEDHALYVEFRPKL
KATPFKFPQLKNHEISFVIANTLVKSNKFETAPTNYNLRVIEVTV
AANALATRYSVALPSHKDNSNSERGNLRDFMDAYYARYENQAQPW
NGDIGTGIERLLKMLQLLVEESFSRKKSGFTVHEASTALNCSREE
FTRDYLTTFPVRFQVLKLYQRAKHVYSESLRVLKALKMMTSATFH
TDEDFFTDFGRLMNESQASCDKLYECSCIETNQICSIALANGSFG
SRLTGAGWGGCTIHLVPSGANGNVEQVRKALIEKFYNVRYPDLTD
EKLKDAIVSXPALGTCLYEQ
14-BC-FP
SEQ ID NO: 9
ACTCACTATAGGGCGAATTGTACTGCAGGTCGACTGGATGGCGGCGTTAG
38-BC-FP
SEQ ID NO: 10
ACTCACTATAGGGCGAATTGGGACTCCTGTCGACTGGATGGCGGCGTTAG
39-BC-FP
SEQ ID NO: 11
ACTCACTATAGGGCGAATTGTAGGCATGGTCGACTGGATGGCGGCGTTAG
40-BC-FP
SEQ ID NO: 12
ACTCACTATAGGGCGAATTGTAAGGCGAGTCGACTGGATGGCGGCGTTAG
41-BC-FP
SEQ ID NO: 13
ACTCACTATAGGGCGAATTGCGTACTAGGTCGACTGGATGGCGGCGTTAG
42-BC-FP
SEQ ID NO: 14
ACTCACTATAGGGCGAATTGTATCCTCTGTCGACTGGATGGCGGCGTTAG
Gal3p^{MC} Nucleotide sequence
SEQ ID NO: 15
atgaatacaaacgttccaatattcagttctccggtcagagattta
ccaaggtctttcgaacaaaaacatttagcggttgtagatgcattt
ttccaaacataccatgtcaaacctgattttatcgctaggtctcct
ggcagagtaaatctgattggtgagcatatagattattgcgatttt
tcagttttgccattagccattaatatggatatgctttgcgcagtt
aaaattttagacgaaaaaaatccatccattaccttaacaaatgcg
gaccctaaatttgctcagcgaaagtttgatctgcctttagatggt
tcctacatggtgatagatccgtctgtgtcggaatggtcgaattac
tttaaatgcggactacatgtggcacattcatacttgaaaaaaatt
gctccggaaagatttaataatacacccttagtaggtgcgcagatc
ttttgccagagcgatattcctactggtggtggactctcatctgca
tttacttgcgcggcagcactagccacaattagagccaatatggga
aaaaattttgatatttccaaaaaagacttgacccgcatcacagcg
gttgctgagcactatgttggagtcaataatggtggtatggatcaa
gcaacgtctgtttatggggaagaagatcatgctctatacgtagag
tttaggccaaaactaaaggccacacctttcaagtatcctcaattg
aaaaatcatgaaatcagtttcgtcatcgccaatactcttgtaaag
tctaataaattcgaaactgctcctacaaattacaatttaagagta
ttagaggtaacagttgctgccaacgccttggcgaccagatacagc
gtggccttaccatctcacaaggacaattctaactcagaaagaggg
aatctaagagattttatggatgcttactacgccagatacgaaaac
caagcccaaccatggaatggagatatcggaactggtattgaacgt
ttactcaagatgctacaattggtagaagaatctttctcgaggaaa
aagagcggattcactgtacatgaagcctctacggcgctaaactgt
tcacgtgaggagttcactagagattacctgacaacttttcccgtc
cgcttccaagtcttgaaactatatcaaagagctaaacacgtttac
tccgaatccttaagggtgcttaaggctttaaaaatgatgacaagt
gccacttttcacacggacgaagatttctttacagattttggccga
ctaatgaatgagtcccaggcctcttgtgataaactttatgaatgt
tcgtgcatagaaaccaatcaaatatgctcgattgccctagcaaat
ggttctttcggctcccgtctcactggcgctggttggggcggttgc
actatccaccttgttcctagtggcgctaatgggaacgtggaacag
gtacgaaaagcactaatcgaaaaattctacaatgtaagatatccg
gatctcacagatgaagaactaaaagacgcaattatagtttcgaag
cctgccttgggtacttgtttgtacgaacaataa
Tal1
SEQ ID NO: 16
MSEPAQKKQKVANNSLEQLKASGTVVVADTGDFGSIAKFQPQDST
TNPSLILAAAKQPTYAKLIDVAVEYGKKHGKTTEEQVENAVDRLL
VEFGKEILKIVPGRVSTEVDARLSFDTQATIEKARHIIKLFEQEG
VSKERVLIKIASTWEGIQAAKELEEKDGIHCNLTLLFSFVQAVAC
AEAQVTLISPFVGRILDWYKSSTGKDYKGEADPGVISVKKIYNYY
KKYGYKTIVMGASFRSTDEIKNLAGVDYLTISPALLDKLMNSTEP
FPRVLDPVSAKKEAGDKISYISDESKFRFDLNEDAMATEKLSEGI
RKFSADIVTLFDLIEKKVTA
Gal2
SEQ ID NO: 17
MAVEENNMPVVSQQPQAGEDVISSLSKDSHLSAQSQKYSNDELKA
GESGSEGSQSVPIEIPKKPMSEYVTVSLLCLCVAFGGFMFGWDTG
TISGFVVQTDFLRRFGMKHKDGTHYLSNVRTGLIVAIFNIGCAFG
GIILSKGGDMYGRKKGLSIVVSVYIVGIIIQIASINKWYQYFIGR
IISGLGVGGIAVLCPMLISEIAPKHLRGTLVSCYQLMITAGIFLG
YCTNYGTKSYSNSVQWRVPLGLCFAWSLFMIGALTLVPESPRYLC
EVNKVEDAKRSIAKSNKVSPEDPAVQAELDLIMAGIEAEKLAGNA
SWGELFSTKTKVFQRLLMGVFVQMFQQLTGNNYFFYYGTVIFKSV
GLDDSFETSIVIGVVNFASTFFSLWTVENLGHRKCLLLGAATMMA
CMVIYASVGVTRLYPHGKSQPSSKGAGNCMIVFTCFYIFCYATTW
APVAWVITAESFPLRVKSKCMALASASNWVWGFLIAFFTPFITSA
INFYYGYVFMGCLVAMFFYVFFFVPETKGLSLEEIQELWEEGVLP
WKSEGWIPSSRRGNNYDLEDLQHDDKPWYKAMLE.
E. coli araA
SEQ ID NO: 18
MTIFDNYEVWFVIGSQHLYGPETLRQVTQHAEHVVNALNTEAKLP
CKLVLKPLGTTPDEITAICRDANYDDRCAGLVVWLHTFSPAKMWI
NGLTMLNKPLLQFHTQFNAALPWDSIDMDFMNLNQTAHGGREFGF
IGARMRQQHAVVTGHWQDKQAHERIGSWMRQAVSKQDTRHLKVCR
FGDNMREVAVTDGDKVAAQIKFGFSVNTWAVGDLVQVVNSISDGD
VNALVDEYESCYTMTPATQIHGKKRQNVLEAARIELGMKRFLEQG
GFHAFTTTFEDLHGLKQLPGLAVQRLMQQGYGFAGEGDWKTAALL
RIMKVMSTGLQGGTSFMEDYTYHFEKGNDLVLGSHMLEVCPSIAA
EEKPILDVQHLGIGGKDDPARLIFNTQTGPAIVASLIDLGDRYRL
LVNCIDTVKTPHSLPKLPVANALWKAQPDLPTASEAWILAGGAHH
TVFSHALNLNDMRQFAEMHDIEITVIDNDTRLPAFKDALRWNEVY
YGFRR
E. coli araB
SEQ ID NO: 19
MAIAIGLDFGSDSVRALAVDCATGEEIATSVEWYPRWQKGQFCDA
PNNQFRHHPRDYIESMEAALKTVLAELSVEQRAAVVGIGVDSTGS
TPAPIDADGNVLALRPEFAENPNAMFVLWKDHTAVEEAEEITRLC
HAPGNVDYSRYIGGIYSSEWFWAKILHVTRQDSAVAQSAASWIEL
CDWVPALLSGTTRPQDIRRGRCSAGHKSLWHESWGGLPPASFFDE
LDPILNRHLPSPLFTDTWTADIPVGTLCPEWAQRLGLPESVVISG
GAFDCHMGAVGAGAQPNALVKVIGTSTCDILIADKQSVGERAVKG
ICGQVDGSVVPGFIGLEAGQSAFGDIYAWFGRVLGWPLEQLAAQH
PELKTQINASQKQLLPALTEAWAKNPSLDHLPVVLDWFNGRRTPN
ANQRLKGVITDLNLATDAPLLFGGLIAATAFGARAIMECFTDQGI
AVNNVMALGGIARKNQVIMQACCDVLNRPLQIVASDQCCALGAAI
FAAVAAKVHADIPSAQQKMASAVEKTLQPCSEQAQRFEQLYRRYQ
QWAMSAEQHYLPTSAPAQAAQAVATL
E. coli araD
SEQ ID NO: 20
MLEDLKRQVLEANLALPKHNLVTLTWGNVSAVDRERGVFVIKPSG
VDYSVMTADDMVVVSIETGEVVEGTKKPSSDTPTHRLLYQAFPSI
GGIVHTHSRHATIWAQAGQSIPATGTTHADYFYGTIPCTRKMTDA
EINGEYEWETGNVIVETFEKQGIDAAQMPGVLVHSHGPFAWGKNA
EDAVHNAIVLEEVAYMGIFCRQLAPQLPDMQQTLLDKHYLRKHGA
KAYYGQ
XYLA from Piromyces
SEQ ID NO: 21
    1 makeyfpqiq kikfegkdsk nplafhyyda ekevmgkkmk dwlrfamaww htlcaegadq
  61 fgggtksfpw negtdaieia kqkvdagfei mqklgipyyc fhdvdlvseg nsieeyesnl
121 kavvaylkek qketgikllw stanvfghkr ymngastnpd fdvvaraivq iknaidagie
181 lgaenyvfwg gregymslln tdqkrekehm atmltmardy arskgfkgtf liepkpmept
241 khqydvdtet aigflkahnl dkdfkvniev nhatlaghtf ehelacavda gmlgsidanr
301 gdyqngwdtd qfpidqyelv qawmeiirgg gfvtggtnfd aktrrnstdl ediiiahvsg
361 mdamaralen aakllqespy tkmkkeryas fdsgigkdfe dgkltleqvy eygkkngepk
421 qtsgkqelye aivamyq
XKS1 from S. cerevisiae
SEQ ID NO: 22
MLCSVIQRQTREVSNTMSLDSYYLGFDLSTQQLKCLAINQDLKIV
HSETVEFEKDLPHYHTKKGVYIHGDTIECPVAMWLEALDLVLSKY
REAKFPLNKVMAVSGSCQQHGSVYWSSQAESLLEQLNKKPEKDLL
HYVSSVAFARQTAPNWQDHSTAKQCQEFEECIGGPEKMAQLTGSR
AHFRFTGPQILKIAQLEPEAYEKTKTISLVSNFLTSILVGHLVEL
EEADACGMNLYDIRERKFSDELLHLIDSSSKDKTIRQKLMRAPMK
NLIAGTICKYFIEKYGFNTNCKVSPMTGDNLATICSLPLRKNDVL
VSLGTSTTVLLVTDKYHPSPNYHLFIHPTLPNHYMGMICYCNGSL
ARERIRDELNKERENNYEKTNDWTLFNQAVLDDSESSENELGVYF
PLGEIVPSVKAINKRVIFNPKTGMIEREVAKFKDKRHDAKNIVES
QALSCRVRISPLLSDSNASSQQRLNEDTIVKFDYDESPLRDYLNK
RPERTFFVGGASKNDAIVKKFAQVIGATKGNFRLETPNSCALGGC
YKAMWSLLYDSNKIAVPFDKFLNDNFPWHVMESISDVDNENWDRY
NSKIVPLSELEKTLI
```

We claim:

1. An engineered protein, wherein the engineered protein is a variant of Gal3p, wherein the variant of Gal3p is fully activated.

2. The engineered protein of claim 1, wherein the variant of Gal3p possesses a conformational change corresponding to galactose-bound Gal3p.

3. The engineered protein of claim 1, wherein the variant of Gal3p comprises SEQ ID NO: 1 or a sequence having at least 80% identity to SEQ ID NO: 1.

4. The engineered protein of claim 1, wherein the variant of Gal3p has at least 80% identity to SEQ ID NO: 1 and comprises D68N, V69M, A109V, F237Y, and I71L substitution mutations relative to SEQ ID NO: 2.

5. The engineered protein of claim 1, wherein the variant of Gal3p comprises SEQ ID NO: 1.

6. The engineered protein of claim 1, wherein the variant of Gal3p consists of SEQ ID NO: 1.

7. The engineered protein of claim 1, wherein the engineered protein activates the galactose regulon.

8. The engineered protein of claim 7, wherein the galactose regulon is activated by indirect action.

9. The engineered protein of claim 1, wherein the engineered protein allows a microbial cell comprising or expressing the engineered protein to grow on a non-native substrate.

10. A nucleic acid construct encoding the engineered protein of claim 1.

11. The nucleic acid construct of claim 10, wherein the nucleic acid construct comprises SEQ ID NO: 15 or a sequence having at least 80% identity to SEQ ID NO: 15.

12. A microbial cell comprising the engineered protein of claim 1.

13. A multicellular microbial organism comprising at least one microbial cell of claim 12.

14. The microbial cell of claim 12, wherein the microbial cell is a yeast.

15. The microbial cell of claim 14, wherein the yeast is Saccharomyces cerevisiae.

16. The microbial cell of claim 12, wherein the microbial cell grows in an inducer-independent manner.

17. A method of engineering a microbial organism for growth on a non-native substrate, wherein the method comprises synthetically activating the GAL response system in the microbial organism.

18. A method of engineering a microbial organism for growth on a non-native substrate, wherein the method consists of synthetically activating the GAL response system in the microbial organism.

19. A method of growing a microbial organism on a non-native substrate, wherein the method comprises expressing the engineered protein of claim 1 in the microbial organism.

20. A method of engineering a microbial organism for growth on a non-native substrate, wherein the method comprises expressing an engineered protein in the microbial organism, wherein the engineered protein is Gal3pMC, wherein the microbial organism is Saccharomyces cerevisiae, and wherein the non-native substrate is selected from the group consisting of arabinose, xylose, cellobiose, and raffinose.


SEQUENCE LISTING (22 sequences)

SEQ ID NO: 1 - protein, 520 aa, synthetic construct
MNTNVPIFSSPVRDLPRSFEQKHLAVVDAFFQTYHVKPDFIARSPGRVNLIGEHIDYCDFSVLPLAINMDMLCAVKILDEKNPSITLTNADPKFAQRKFDLPLDGSYMVIDPSVSEWSNYFKCGLHVAHSYLKKIAPERFNNTPLVGAQIFCQSDIPTGGGLSSAFTCAAALATIRANMGKNFDISKKDLTRITAVAEHYVGVNNGGMDQATSVYGEEDHALYVEFRPKLKATPFKYPQLKNHEISFVIANTLVKSNKFETAPTNYNLRVLEVTVAANALATRYSVALPSHKDNSNSERGNLRDFMDAYYARYENQAQPWNGDIGTGIERLLKMLQLVEESFSRKKSGFTVHEASTALNCSREEFTRDYLTTFPVRFQVLKLYQRAKHVYSESLRVLKALKMMTSATFHTDEDFFTDFGRLMNESQASCDKLYECSCIETNQICSIALANGSFGSRLTGAGWGGCTIHLVPSGANGNVEQVRKALIEKFYNVRYPDLTDEELKDAIIVSKPALGTCLYEQ

SEQ ID NO: 2 - protein, 520 aa, Saccharomyces cerevisiae
MNTNVPIFSSPVRDLPRSFEQKHLAVVDAFFQTYHVKPDFIARSPGRVNLIGEHIDYCDFSVLPLAIDVDMLCAVKILDEKNPSITLTNADPKFAQRKFDLPLDGSYMAIDPSVSEWSNYFKCGLHVAHSYLKKIAPERFNNTPLVGAQIFCQSDIPTGGGLSSAFTCAAALATIRANMGKNFDISKKDLTRITAVAEHYVGVNNGGMDQATSVYGEEDHALYVEFRPKLKATPFKFPQLKNHEISFVIANTLVKSNKFETAPTNYNLRVIEVTVAANALATRYSVALPSHKDNSNSERGNLRDFMDAYYARYENQAQPWNGDIGTGIERLLKMLQLVEESFSRKKSGFTVHEASTALNCSREEFTRDYLTTFPVRFQVLKLYQRAKHVYSESLRVLKALKMMTSATFHTDEDFFTDFGRLMNESQASCDKLYECSCIETNQICSIALANGSFGSRLTGAGWGGCTIHLVPSGANGNVEQVRKALIEKFYNVRYPDLTDEELKDAIIVSKPALGTCLYEQ

SEQ ID NO: 3 - protein, 520 aa, synthetic construct
MNTNVPIFSSPVRDLPRSFEQKHLAVVDAFFQTYHVKPDFIARSPGRVNLIGEHIDYCDFSVLPLAINMDMLCAVKILDEKNPSITLTNADPKFAQRKFDLPLDGSYMVIDPSVSEWSNYFKCGLHVAHSYLKKIAPERFNNTPLVGAQIFCQSDIPTGGGLSSAFTCAAALATIRANMGKNFDISKKDLTRITAVAEHYVGVNNGGMDQATSVYGEEDHALYVEFRPKLKATPFKFPQLKNHEISFVIANTLVKSNKFETAPTNYNLRVLEVTVAANALATRYSVALPSHKDNSNSERGNLRDFMDAYYARYENQAQPWNGDIGTGIERLLKMLQLVEESFSRKKSGFTVHEASTALNCSREEFTRDYLTTFPVRFQVLKLYQRAKHVYSESLRVLKALKMMTSATFHTDEDFFTDFGRLMNESQASCDKLYECSCIETNQICSIALANGSFGSRLTGAGWGGCTIHLVPSGANGNVEQVRKALIEKFYNVRYPDLTDEELKDAIIVSKPALGTCLYEQ

SEQ ID NO: 4 - protein, 520 aa, synthetic construct
MNTNVPIFSSPVRDLPRSFEQKHLAVVDAFFQTYHVKPDFIARSPGRVNLIGEHIDYCDFSVLPLAIDVDMLCAVKILDEKNPSITLTNADPKFAQRKFDLPLDGSYMAIDPSVSEWSNYFKCGLHVAHSYLKKIAPERFNNTPLVGAQIFCQSDIPTGGGLSSAFTCAAALATIRANMGKNFDISKKDLTRITAVAEHYVGVNNGGMDQATSVYGEEDHALYVEFRPKLKATPFKFPQLKNHEISFVIANTLVKSNKFETAPTNYNLRVIEVTVAANALATRYSVALPSHKDNSNSERGNLRDFMDAYYARYENQAQPWNGDIGTGIERLLKMLQLVEESFSRKKSGFTVHEASTALNCSREEFTRDYLTTFPVRFQVLKLYQRAKHVYSESLRVLKALKMMTSATFHTDEDFFTDFGRLMNESQASCDKLYECSCIETNQICSIALANGSFGSRLTGAGWGGCTIHLVPSGANGNVEQVRKALIEKFYNVRYPDLTDEELKDAIIVPKPALGTCLYEQ

SEQ ID NO: 5 - protein, 520 aa, synthetic construct
MNTNVPIFSSPVRDLPRSFEQKHLAVVDAFFQTYHVKPDFIARSPGRVNLIGEHIDYCDFSVLPLAIDVDMLCAVKILDEKNPSITLTNADPKFAQRKFDLPLDGSYMAIDPSVSEWSNYFKCGLHVAHSYLKKIAPERFNNTPLVGAQIFCQSDIPTGGGLSSAFTCAAALATIRANMGKNFDISKKDLTRITAVAEHYVGVNNGGMDQATSVYGEEDHALYVEFRPKLKATPFKYPQLKNHEISFVIANTLVKSNKFETAPTNYNLRVIEVTVAANALATRYSVALPSHKDNSNSERGNLRDFMDAYYARYENQAQPWNGDIGTGIERLLKMLQLVEESFSRKKSGFTVHEASTALNCSREEFTRDYLTTFPVRFQVLKLYQRAKHVYSESLRVLKALKMMTSATFHTDEDFFTDFGRLMNESQASCDKLYECSCIETNQICSIALANGSFGSRLTGAGWGGCTIHLVPSGANGNVEQVRKALIEKFYNVRYPDLTDEELKDAIIVSKPALGTCLYEQ

SEQ ID NO: 6 - protein, 520 aa, synthetic construct
MNTNVPIFSSPVRDLPRSFEQKHLAVVDAFFQTYHVKPDFIARSPGRVNLIGEHIDYCDFSVLPLAIDVDMLCAVKILDEKNPSITLTNADPKFAQRKFDLPLDGSYMAIDPSVSEWSNYFKCGLHVAHSYLKKIAPERFNNTPLVGAQIFCQSDIPTGGGLSSAFTCAAALATIRANMGKNFDISKKDLTRITAVAEHYVGVNNGGMDQATSVYGEEDHALYVEFRPKLKATPFKYPQLKNHEISFVIANTLVKSNKFETAPTNYNLRVIEVTVAANALATRYSVALPSHKDNSNSERGNLRDFMDAYYARYENQAQPWNGDIGTGIERLLKMLQLVEESFSRKKSGFTVHEASTALNCSREEFTRDYLTTFPVRFQVLKLYQRAKHVYSESLRVLKALKMMTSATFHTDEDFFTDFGRLMNESQASCDKLYECSCIETNQICSIALANGSFGSRLTGAGWGGCTIHLVPSGANGNVEQVRKALIEKFYNVRYPDLTDEELKDAIIVPKPALGTCLYEQ

SEQ ID NO: 7 - protein, 521 aa, synthetic construct
SNTNVPIFSSPVRDLPRSFEQKHLAVVDAFFQTYHVKPDFIARSPGRVNLIGEHIDYCDFSVLPLAIDVDMLCAVKILDEKNPSITLTNADPKFAAQRKFDLPLDGSYMAIDPSVSEWSNYFXCGLHVAHSYLKKIAPERFNNTPLVGAQIFCQSDIPTGGGLSSAFTCAAALATIRANMGXNFDISKKDLTRITAVAEHYVGVNNGGMDQATSVYGEEDHALYVEFRPKLKATPFKFPQLKNHEISFVIANTLVKSNKFETAPTNYNLRVIEVTVAANALATRYSVALPSHKDNSNSERGNLRDFMDAYYARYENQAQPWNGDIGTGIERLLKMLQLLVEESFSRKKSGFTVHEASTALNCSREEFTRDYLTTFPVRFQVLKLYQRAKHVYSESLRVLKALKMMTSATFHTDEDFFTDFGRLMNESQASCDKLYECSCIETNQICSIALANGSFGSRLTGAGWGGCTIHLVPSGANGNVEQVRKALIEKFYNVRYPDLTDEKLKDAIVSXPALGTCLYEQ

SEQ ID NO: 8 - protein, 515 aa, synthetic construct
NTNVPIFSSPRSFEQKHLAVVDAFFQTYHVKPDFIARSPGRVNLIGEHIDYCDFSVLPLAIDVDMLCAVKILDEKNPSITLTNADPKFAAQRKFDLPLDGSYMAIDPSVSEWSNYFXCGLHVAHSYLKKIAPERFNNTPLVGAQIFCQSDIPTGGGLSSAFTCAAALATIRANMGXNFDISKKDLTRITAVAEHYVGVNNGGMDQATSVYGEEDHALYVEFRPKLKATPFKFPQLKNHEISFVIANTLVKSNKFETAPTNYNLRVIEVTVAANALATRYSVALPSHKDNSNSERGNLRDFMDAYYARYENQAQPWNGDIGTGIERLLKMLQLLVEESFSRKKSGFTVHEASTALNCSREEFTRDYLTTFPVRFQVLKLYQRAKHVYSESLRVLKALKMMTSATFHTDEDFFTDFGRLMNESQASCDKLYECSCIETNQICSIALANGSFGSRLTGAGWGGCTIHLVPSGANGNVEQVRKALIEKFYNVRYPDLTDEKLKDAIVSXPALGTCLYEQ

SEQ ID NO: 9 - DNA, 50 bp, synthetic construct
  source 1..50: mol_type = other DNA
actcactatagggcgaattgtactgcaggtcgactggatggcggcgttag

SEQ ID NO: 10 - DNA, 50 bp, synthetic construct
  source 1..50: mol_type = other DNA
actcactatagggcgaattgggactcctgtcgactggatggcggcgttag

SEQ ID NO: 11 - DNA, 50 bp, synthetic construct
  source 1..50: mol_type = other DNA
actcactatagggcgaattgtaggcatggtcgactggatggcggcgttag

SEQ ID NO: 12 - DNA, 50 bp, synthetic construct
  source 1..50: mol_type = other DNA
actcactatagggcgaattgtaaggcgagtcgactggatggcggcgttag

SEQ ID NO: 13 - DNA, 50 bp, synthetic construct
  source 1..50: mol_type = other DNA
actcactatagggcgaattgcgtactaggtcgactggatggcggcgttag

SEQ ID NO: 14 - DNA, 50 bp, synthetic construct
  source 1..50: mol_type = other DNA
actcactatagggcgaattgtatcctctgtcgactggatggcggcgttag

SEQ ID NO: 15 - DNA, 1563 bp, synthetic construct
  source 1..1563: mol_type = other DNA
atgaatacaaacgttccaatattcagttctccggtcagagatttaccaaggtctttcgaacaaaaacatttagcggttgtagatgcatttttccaaacataccatgtcaaacctgattttatcgctaggtctcctggcagagtaaatctgattggtgagcatatagattattgcgatttttcagttttgccattagccattaatatggatatgctttgcgcagttaaaattttagacgaaaaaaatccatccattaccttaacaaatgcggaccctaaatttgctcagcgaaagtttgatctgcctttagatggttcctacatggtgatagatccgtctgtgtcggaatggtcgaattactttaaatgcggactacatgtggcacattcatacttgaaaaaaattgctccggaaagatttaataatacacccttagtaggtgcgcagatcttttgccagagcgatattcctactggtggtggactctcatctgcatttacttgcgcggcagcactagccacaattagagccaatatgggaaaaaattttgatatttccaaaaaagacttgacccgcatcacagcggttgctgagcactatgttggagtcaataatggtggtatggatcaagcaacgtctgtttatggggaagaagatcatgctctatacgtagagtttaggccaaaactaaaggccacacctttcaagtatcctcaattgaaaaatcatgaaatcagtttcgtcatcgccaatactcttgtaaagtctaataaattcgaaactgctcctacaaattacaatttaagagtattagaggtaacagttgctgccaacgccttggcgaccagatacagcgtggccttaccatctcacaaggacaattctaactcagaaagagggaatctaagagattttatggatgcttactacgccagatacgaaaaccaagcccaaccatggaatggagatatcggaactggtattgaacgtttactcaagatgctacaattggtagaagaatctttctcgaggaaaaagagcggattcactgtacatgaagcctctacggcgctaaactgttcacgtgaggagttcactagagattacctgacaacttttcccgtccgcttccaagtcttgaaactatatcaaagagctaaacacgtttactccgaatccttaagggtgcttaaggctttaaaaatgatgacaagtgccacttttcacacggacgaagatttctttacagattttggccgactaatgaatgagtcccaggcctcttgtgataaactttatgaatgttcgtgcatagaaaccaatcaaatatgctcgattgccctagcaaatggttctttcggctcccgtctcactggcgctggttggggcggttgcactatccaccttgttcctagtggcgctaatgggaacgtggaacaggtacgaaaagcactaatcgaaaaattctacaatgtaagatatccggatctcacagatgaagaactaaaagacgcaattatagtttcgaagcctgccttgggtacttgtttgtacgaacaataa

SEQ ID NO: 16 - protein, 335 aa, Saccharomyces cerevisiae
MSEPAQKKQKVANNSLEQLKASGTVVVADTGDFGSIAKFQPQDSTTNPSLILAAAKQPTYAKLIDVAVEYGKKHGKTTEEQVENAVDRLLVEFGKEILKIVPGRVSTEVDARLSFDTQATIEKARHIIKLFEQEGVSKERVLIKIASTWEGIQAAKELEEKDGIHCNLTLLFSFVQAVACAEAQVTLISPFVGRILDWYKSSTGKDYKGEADPGVISVKKIYNYYKKYGYKTIVMGASFRSTDEIKNLAGVDYLTISPALLDKLMNSTEPFPRVLDPVSAKKEAGDKISYISDESKFRFDLNEDAMATEKLSEGIRKFSADIVTLFDLIEKKVTA

SEQ ID NO: 17 - protein, 574 aa, Saccharomyces cerevisiae
MAVEENNMPVVSQQPQAGEDVISSLSKDSHLSAQSQKYSNDELKAGESGSEGSQSVPIEIPKKPMSEYVTVSLLCLCVAFGGFMFGWDTGTISGFVVQTDFLRRFGMKHKDGTHYLSNVRTGLIVAIFNIGCAFGGIILSKGGDMYGRKKGLSIVVSVYIVGIIIQIASINKWYQYFIGRIISGLGVGGIAVLCPMLISEIAPKHLRGTLVSCYQLMITAGIFLGYCTNYGTKSYSNSVQWRVPLGLCFAWSLFMIGALTLVPESPRYLCEVNKVEDAKRSIAKSNKVSPEDPAVQAELDLIMAGIEAEKLAGNASWGELFSTKTKVFQRLLMGVFVQMFQQLTGNNYFFYYGTVIFKSVGLDDSFETSIVIGVVNFASTFFSLWTVENLGHRKCLLLGAATMMACMVIYASVGVTRLYPHGKSQPSSKGAGNCMIVFTCFYIFCYATTWAPVAWVITAESFPLRVKSKCMALASASNWVWGFLIAFFTPFITSAINFYYGYVFMGCLVAMFFYVFFFVPETKGLSLEEIQELWEEGVLPWKSEGWIPSSRRGNNYDLEDLQHDDKPWYKAMLE

SEQ ID NO: 18 - protein, 500 aa, Escherichia coli
MTIFDNYEVWFVIGSQHLYGPETLRQVTQHAEHVVNALNTEAKLPCKLVLKPLGTTPDEITAICRDANYDDRCAGLVVWLHTFSPAKMWINGLTMLNKPLLQFHTQFNAALPWDSIDMDFMNLNQTAHGGREFGFIGARMRQQHAVVTGHWQDKQAHERIGSWMRQAVSKQDTRHLKVCRFGDNMREVAVTDGDKVAAQIKFGFSVNTWAVGDLVQVVNSISDGDVNALVDEYESCYTMTPATQIHGKKRQNVLEAARIELGMKRFLEQGGFHAFTTTFEDLHGLKQLPGLAVQRLMQQGYGFAGEGDWKTAALLRIMKVMSTGLQGGTSFMEDYTYHFEKGNDLVLGSHMLEVCPSIAAEEKPILDVQHLGIGGKDDPARLIFNTQTGPAIVASLIDLGDRYRLLVNCIDTVKTPHSLPKLPVANALWKAQPDLPTASEAWILAGGAHHTVFSHALNLNDMRQFAEMHDIEITVIDNDTRLPAFKDALRWNEVYYGFRR

SEQ ID NO: 19 - protein, 566 aa, Escherichia coli
MAIAIGLDFGSDSVRALAVDCATGEEIATSVEWYPRWQKGQFCDAPNNQFRHHPRDYIESMEAALKTVLAELSVEQRAAVVGIGVDSTGSTPAPIDADGNVLALRPEFAENPNAMFVLWKDHTAVEEAEEITRLCHAPGNVDYSRYIGGIYSSEWFWAKILHVTRQDSAVAQSAASWIELCDWVPALLSGTTRPQDIRRGRCSAGHKSLWHESWGGLPPASFFDELDPILNRHLPSPLFTDTWTADIPVGTLCPEWAQRLGLPESVVISGGAFDCHMGAVGAGAQPNALVKVIGTSTCDILIADKQSVGERAVKGICGQVDGSVVPGFIGLEAGQSAFGDIYAWFGRVLGWPLEQLAAQHPELKTQINASQKQLLPALTEAWAKNPSLDHLPVVLDWFNGRRTPNANQRLKGVITDLNLATDAPLLFGGLIAATAFGARAIMECFTDQGIAVNNVMALGGIARKNQVIMQACCDVLNRPLQIVASDQCCALGAAIFAAVAAKVHADIPSAQQKMASAVEKTLQPCSEQAQRFEQLYRRYQQWAMSAEQHYLPTSAPAQAAQAVATL

SEQ ID NO: 20 - protein, 231 aa, Escherichia coli
MLEDLKRQVLEANLALPKHNLVTLTWGNVSAVDRERGVFVIKPSGVDYSVMTADDMVVVSIETGEVVEGTKKPSSDTPTHRLLYQAFPSIGGIVHTHSRHATIWAQAGQSIPATGTTHADYFYGTIPCTRKMTDAEINGEYEWETGNVIVETFEKQGIDAAQMPGVLVHSHGPFAWGKNAEDAVHNAIVLEEVAYMGIFCRQLAPQLPDMQQTLLDKHYLRKHGAKAYYGQ

SEQ ID NO: 21 - protein, 437 aa, synthetic construct
MAKEYFPQIQKIKFEGKDSKNPLAFHYYDAEKEVMGKKMKDWLRFAMAWWHTLCAEGADQFGGGTKSFPWNEGTDAIEIAKQKVDAGFEIMQKLGIPYYCFHDVDLVSEGNSIEEYESNLKAVVAYLKEKQKETGIKLLWSTANVFGHKRYMNGASTNPDFDVVARAIVQIKNAIDAGIELGAENYVFWGGREGYMSLLNTDQKREKEHMATMLTMARDYARSKGFKGTFLIEPKPMEPTKHQYDVDTETAIGFLKAHNLDKDFKVNIEVNHATLAGHTFEHELACAVDAGMLGSIDANRGDYQNGWDTDQFPIDQYELVQAWMEIIRGGGFVTGGTNFDAKTRRNSTDLEDIIIAHVSGMDAMARALENAAKLLQESPYTKMKKERYASFDSGIGKDFEDGKLTLEQVYEYGKKNGEPKQTSGKQELYEAIVAMYQ

SEQ ID NO: 22 - protein, 600 aa, Saccharomyces cerevisiae
MLCSVIQRQTREVSNTMSLDSYYLGFDLSTQQLKCLAINQDLKIVHSETVEFEKDLPHYHTKKGVYIHGDTIECPVAMWLEALDLVLSKYREAKFPLNKVMAVSGSCQQHGSVYWSSQAESLLEQLNKKPEKDLLHYVSSVAFARQTAPNWQDHSTAKQCQEFEECIGGPEKMAQLTGSRAHFRFTGPQILKIAQLEPEAYEKTKTISLVSNFLTSILVGHLVELEEADACGMNLYDIRERKFSDELLHLIDSSSKDKTIRQKLMRAPMKNLIAGTICKYFIEKYGFNTNCKVSPMTGDNLATICSLPLRKNDVLVSLGTSTTVLLVTDKYHPSPNYHLFIHPTLPNHYMGMICYCNGSLARERIRDELNKERENNYEKTNDWTLFNQAVLDDSESSENELGVYFPLGEIVPSVKAINKRVIFNPKTGMIEREVAKFKDKRHDAKNIVESQALSCRVRISPLLSDSNASSQQRLNEDTIVKFDYDESPLRDYLNKRPERTFFVGGASKNDAIVKKFAQVIGATKGNFRLETPNSCALGGCYKAMWSLLYDSNKIAVPFDKFLNDNFPWHVMESISDVDNENWDRYNSKIVPLSELEKTLI